(19) United States
(12) Patent Application Publication
(10) Pub. No.: US 2003/0235561 A1
(43) Pub. Date: Dec. 25, 2003

(54) Vascularized organized tissues and uses thereof

(75) Inventors: Herman H. Vandenburgh, Providence, RI (US); Robert F. Valentini, Cranston, RI (US); Xiao Wang, Providence, RI (US); Janet Shansky, Barrington, RI (US); Paulette Ferland, Tiverton, RI (US); Michael DelTatto, Bristol, RI (US)

Correspondence Address: PALMER & DODGE, LLP, KATHLEEN M. WILLIAMS, 111 HUNTINGTON AVENUE, BOSTON, MA, 02199, US

(73) Assignee: Cell Based Delivery Inc.

(21) Appl. No.: 10/281,765

(22) Filed: Oct. 28, 2002

Related U.S. Application Data
(60) Provisional application No. 60/391,330, filed on Jun. 25, 2002.
(60) Provisional application No. 60/399,605, filed on Jul. 30, 2002.

(51) Int. Cl. A61K 48/00; C12N 5/08; C12N 15/85

(52) U.S. Cl. 424/93.21; 435/455; 435/366

(57) ABSTRACT

The invention relates to organized tissues that are implanted into an organism wherein they become vascularized. The invention also relates to methods of using an organized tissue that is vascularized following implantation into an organism, for delivery of a bioactive compound. The invention also relates to methods of producing an organized tissue that is vascularized following implantation into an organism.

### PRIORITY INFORMATION

This invention claims priority to U.S. Ser. No. 60/391,330, filed Jun. 25, 2002 and U.S. Ser. No. 60/399,605, filed Jul. 30, 2002.

### FIELD OF THE INVENTION

The invention relates to the preparation and use of organized tissues that are vascularized following implantation into an organism.

### BACKGROUND OF THE INVENTION

This invention relates to the delivery of bioactive compounds to an organism, and in particular to methods and apparatus for the delivery of bioactive compounds by implanting into the organism an organized tissue that produces the compounds, wherein the organized tissue becomes vascularized following implantation into the organism.

One of the primary therapies used to treat disease is the delivery of bioactive compounds to the affected organism. Bioactive compounds may be delivered systemically or locally by a wide of variety of methods. For example, an exogenous source (i.e., produced outside the organism treated) of the bioactive compound may be provided intermittently by repeated doses. The route of administration may include oral consumption, injection, or tissue absorption via topical compositions, suppositories, inhalants, or the like. Exogenous sources of the bioactive compound may also be provided continuously over a defined time period. For example, delivery systems such as pumps, time-released compositions, or the like may be implanted into the organism on a semi-permanent basis for the administration of bioactive compounds (e.g., insulin, estrogen, progesterone, etc.).

The delivery of bioactive compounds from an endogenous source (i.e., produced within the organism treated) has also been attempted. Traditionally, this was accomplished by transplanting, from another organism, an organ or tissue whose normal physiological function was the production of the bioactive compound (e.g., liver transplantation, kidney transplantation, or the like). More recently, endogenous production by cells of the affected organism has been accomplished by inserting into the cells a DNA sequence which mediates the production of the bioactive compound. Commonly known as gene therapy, this method includes inserting the DNA sequence into the cells of the organism in vivo. The DNA sequence persists either transiently or permanently as an extra-chromosomal vector (e.g., when inserted by adenovirus infection or by direct injection of a plasmid) or integrates into the host cell genome (e.g., when inserted by retrovirus infection). Alternatively, the DNA sequence may be inserted into cells of the host tissue or another organism in vitro, and the cells subsequently transplanted into the organism to be treated.

It is an object of the invention to prepare an organized tissue that becomes vascularized following implantation of the organized tissue into an organism.

It is also an object of the invention to use an organized tissue that becomes vascularized following implantation of the organized tissue into an organism for the delivery of one of more bioactive compounds to an organism.

It is also an object of the invention to use an organized tissue that becomes vascularized following implantation of the organized tissue into an organism for treating disease.

### SUMMARY OF THE INVENTION

In general, the invention features a method of delivering a bioactive compound to an organism using a vascularized organized tissue as the delivery vehicle. The method includes the steps of growing a plurality of cells in vitro under conditions that allow the formation of an organized tissue, and implanting the cells into the organism, whereby the organized tissue becomes vascularized. In certain embodiments, at least a subset of the cells contains a bioactive compound, and the bioactive compound is produced and delivered to the organism upon implantation and vascularization of the organized tissue.

An organized tissue that is vascularized following implantation into an organism is prepared by producing an organized tissue from a plurality of cells, wherein at least a subpopulation of cells comprises one or more DNA sequences encoding either an endogenous or exogenous vasculogenic factor. The invention provides for cells wherein the sequence encoding the vasculogenic factor is under the control of a promoter. The invention also provides for an organized tissue comprising a plurality of cells, a subset of which comprise a DNA sequence encoding a compound that increases the expression of an endogenous gene encoding a vasculogenic factor. Alternatively, an organized tissue that is vascularized following implantation into an organism is produced from a subpopulation of cells that are mixed with at least one vasculogenic factor. The invention also provides for an organized tissue that is vascularized following implantation, wherein the organized tissue is implanted and at least one vasculogenic factor is administered to the wound site.

The invention also provides for an organized tissue formed n the absence of a vasculogenic factor wherein vascularization is stimulated in a manner including but not limited to the following:

1) the organized tissue is implanted in a highly vascular site of the body (e.g., near or around large blood vessels or vascular networks, or in or near the omentum);

2) the organized tissue is implanted in a site previously or simultaneously treated to stimulate local vascularization (e.g. by using lasers (such as those known in the art for myocardial revascularization), punches or tissue “scoring” with surgical instruments);

3) the organized tissue is implanted with a biomaterial or device (e.g. a degradable polymer or braided silk suture) that stimulates local vascularization; or

4) the organized tissue is comprised of cells (e.g. allogeneic cells) or components (e.g. certain collagens or fibrins) which stimulate a local inflammatory response leading to vascularization.

In one embodiment, the invention provides for a method of delivering a bioactive compound to an organism comprising the following steps. A plurality of cells; wherein at least a subset of cells comprises a DNA sequence selected from the group consisting of a DNA sequence encoding a vasculogenic factor, a DNA sequence encoding a vasculogenic factor operably linked to a promoter and a DNA sequence encoding a factor that increases the expression of a vasculogenic factor, and at least a subset of cells comprises a bioactive compound to be delivered to the organism are grown in vitro. The cells are mixed with an extracellular matrix to create a suspension. The suspension is placed in a vessel having a three-dimensional geometry approximating the in vivo gross morphology of a tissue of interest, wherein the vessel has attachment surfaces. The suspension is allowed to coalesce; and is cultured under conditions in which the cells connect to the attachment surfaces and form an organized tissue having an in vivo-like gross and cellular morphology that is retained upon retrieval of the organized tissue. The tissue is implanted into the organism, wherein the organized tissue becomes vascularized; and whereby the bioactive compound is produced and delivered to the organism. The bioactive compound is of a type or produced in an amount not produced by the tissue of interest, and is produced sufficiently to provide a therapeutic effect to the organism upon implantation of the organized tissue into the organism.

In another embodiment, the organized tissue is comprised of substantially post-mitotic cells.

In another embodiment the organized tissue has an in vivo-like gross and cellular morphology of the tissue of interest.

In another embodiment, the vasculogenic factor is selected from the group consisting of: VEGF A, VEGF B, VEGF C, VEGF D, VEGF E, VEGF F, FGF 1, FGF 2, FGF 3, FGF 4, FGF-5, PDGF AA, PDGF BB, PDGF AB, angiopoeitin, MCP, EPO, IL-1, IL-2, IL-3, IL-4, IL-S, IL-6, IL-7, IL-8, IL-9, IL-10, IL-11, IL-12, IL-13, IL-14, IL-15, IL-16, IL-17, IL-18, IL-19, IL-20, IL-21, IL-22

In another embodiment, implantation is performed by a subcutanteous method.

In another embodiment, the method further comprises the steps of removing the organized tissue from the organism to terminate delivery of the bioactive compound.

In another embodiment, the method further comprises, following the removal step the step of: culturing the tissue in vitro under conditions which preserve its in vivo viability.

In another embodiment, the method further comprises, following the culturing step: the step of: reimplanting the tissue into the organism to deliver the bioactive compound to the organism.

In another embodiment, the tissue is implanted into the tissue of origin of at least one of the cells.

In another embodiment, at least a subset of cells comprises a DNA sequence that mediates the production of two proteins.

In another embodiment, the bioactive compound is a protein.

In another embodiment, the protein is a growth factor.

In another embodiment, the protein is unstable.

In another embodiment, the protein is an antibody.

In another embodiment, the protein is a vaccine.

In another embodiment, the protein is an anti-infectious agent.

In another embodiment, the protein is an anti-inflammatory agent.

In another embodiment, the protein is an anti-adhesion agent.

In another embodiment, the protein is an anti-clotting agent.

In another embodiment, the protein is Factor VIII. The invention encompasses both full-length proteins and proteins wherein at least a portion of the B domain of Factor VIII is deleted. For example, the invention contemplates a Factor VIII protein wherein at least amino acids 746-1640 are deleted.

In another embodiment, the protein is Factor IX.

In another embodiment, the protein is small, for example an interferon, or a cytokine.

The invention facilitates the delivery of proteins, for example Factor VIII, wherein the protein is large, as defined herein, insoluble, poorly absorbed and unstable, as defined herein.

In another embodiment, the organized tissue is comprised of at least one of a cell type selected from the group consisting of: skeletal muscle cells, myoblasts, myofibers, fibroblasts, endothelial cells, smooth muscle cells, cardiac myocytes, osteoblasts, neuronal cells, hepatocytes, mesenchymal stem cells, marrow-derived stem cells, adult stem cells and embryonic stem cells

In another embodiment, during said growing step, a force is exerted parallel to a dimension of the tissue.

In another embodiment, a force is exerted on the individual cells during growth in vitro and on the organized tissue during implantation in vivo.

In another embodiment, the tissue comprises skeletal muscle.

In another embodiment, the tissue comprises myotubes.

In another embodiment, the cells comprise myofibers.

In another embodiment, the organism is a mammal.

In another embodiment, the mammal is a human.

The invention also provides for a method of delivering a bioactive compound to an organism comprising the following steps. A plurality of cells are grown in vitro. At least a subset of cells comprises a bioactive compound to be delivered to the organism. The cells are mixed with an extracellular matrix to create a suspension, and further mixed with at least one vasculogenic factor. The suspension is placed in a vessel having a three-dimensional geometry approximating the in vivo gross morphology of a tissue of interest, and having attachment surfaces. The suspension is allowed to coalesce and is cultured under conditions in which the cells connect to the attachment surfaces and form an organized tissue having an in vivo-like gross and cellular morphology that is retained upon retrieval of the organized tissue. The tissue is implanted into the organism, wherein the organized tissue becomes vascularized; and whereby the bioactive compound is produced and delivered to the organism. The bioactive compound is of a type or produced in an amount not produced by the tissue of interest, and is produced sufficiently to provide a therapeutic effect to the organism upon implantation of theorganized tissue into the organism.

The invention also provides for a method of delivering a bioactive compound to an organism comprising the following steps. A plurality of cells wherein at least a subset of cells comprises a bioactive compound to be delivered to the organism is grown in vitro. The cells are mixed with an extracellular matrix to create a suspension and the suspension is placed in a vessel having a three-dimensional geometry approximating the in vivo gross morphology of a tissue of interest, and having attachment surfaces. The suspension is allowed to coalesce and is cultured under conditions in which the cells connect to the attachment surfaces and form an organized tissue having an in vivo-like gross and cellular morphology that is retained upon retrieval of the organized tissue. The tissue is implanted into the organism and at least one vasculogenic factor is added to the organism, wherein the organized tissue becomes vascularized; and whereby the bioactive compound is produced and delivered to the organism. The bioactive compound is of a type or produced in an amount not produced by the tissue of interest, and is produced sufficiently to provide a therapeutic effect to the organism upon implantation of the organized tissue into the organism.

The invention also provides for a method of delivering a bioactive compound to an organism comprising the following steps. A plurality of cells is grown in vitro. At least a subset of cells comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor. At least a subset of the cells comprises a bioactive compound. The cells are mixed with an extracellular matrix to create a suspension and the suspension is placed s in a vessel wherein the cells form an organized tissue of interest having a three dimensional cellular organization which is retained upon implantation into the organism. The organized tissue is implanted into the organism, whereby the organized tissue is vascularized; and wherein the bioactive compound is produced and delivered to the organism sufficiently to provide a therapeutic effect to the organism. The bioactive compound is of a type or produced in an amount not produced by the tissue of interest.

The invention also provides for a method of delivering a bioactive compound to an organism comprising the following steps. A plurality of cells, wherein at least a subset of cells comprises a bioactive compound are grown in vitro. The cells are mixed with an extracellular matrix to create a suspension and further mixed with at least one vasculogenic factor. The suspension is placed in a vessel wherein the cells form an organized tissue of interest having a three dimensional cellular organization which is retained upon implantation into the organism; The organized tissue is implanted into the organism, whereby the organized tissue is vascularized; and wherein the bioactive compound is produced and delivered to the organism sufficiently to provide a therapeutic effect to theorganism. The bioactive compound is of a type or produced in an amount not produced by the tissue of interest.

The invention also provides for a method of delivering a bioactive compound to an organism comprising the following steps. A plurality of cells is grown in vitro. At least a subset of cells comprises a bioactive compound. The cells are mixed with an extracellular matrix to create a suspension. The suspension is placed in a vessel wherein the cells form an organized tissue of interest having a three dimensional cellular organization which is retained upon implantation into the organism. The organized tissue is implanted into the organism, and at least one vasculogenic factor is added to the organism, whereby the organized tissue is vascularized. The bioactive compound is produced and delivered to the organism sufficiently to provide a therapeutic effect to the organism, whereby the bioactive compound is of a type or produced in an amount not produced by the tissue of interest.

The invention also provides for a method of providing a bioactive compound to an organism in therapeutic need thereof comprising: implanting into the organism an organized tissue having a three-dimensional geometry that is retained upon retrieval of said organized tissue. According to this method, at least a subset of cells comprise a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor, and at least a subset of cells of the organized tissue comprises a bioactive compound to be delivered to the organism. The bioactive compound is produced sufficiently to provide a therapeutic effect to the organism upon implantation of the organized tissue into the organism, and the implanted organized tissue is vascularized.

The invention also provides a method of providing a bioactive compound to an organism in therapeutic need thereof comprising: implanting into the organism an organized tissue having a three-dimensional geometry that is retained upon retrieval of the organized tissue, wherein at least a subset of cells comprise a bioactive compound to be delivered to the organism. The organized tissue is produced by mixing the cells with an extracellular matrix to create a suspension, and further mixing the cells with at least one vasculogenic factor. The bioactive compound is produced sufficiently to provide a therapeutic effect to the organism upon implantation of the organized tissue into the organism and, the implanted organized tissue is vascularized.

The invention also provides for a method of providing a bioactive compound to an organism in therapeutic need thereof comprising implanting into the organism an organized tissue having a three-dimensional geometry that is retained upon retrieval of the organized tissue. At least a subset of cells comprises a bioactive compound to be delivered to the organism, and at least one vasculogenic factor is added to the organism following implantation. The implanted organized tissue is vascularized, and the bioactive compound is produced sufficiently to provide a therapeutic effect to the organism upon implantation of the organized tissue into the organism.

The invention also provides a method of providing a bioactive compound to an organism in therapeutic need thereof comprising implanting into the organism an organized tissue having a three-dimensional geometry approximating the in vivo gross morphology of a tissue of interest that is retained upon implantation of the organized tissue into the organism. At least a subset of the cells of the organized tissue comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor. At least a subset of the cells of the organized tissue comprises a bioactive compound, and the bioactive compound is produced sufficiently to provide a therapeutic effect to the organism upon implantation of the organized tissue into the organism. The implanted organized tissue is vascularized.

The invention also provides a method of providing a bioactive compound to an organism in therapeutic need thereof comprising implanting into the organism an organized tissue having a three-dimensional geometry approximating the in vivo gross morphology of a tissue of interest that is retained upon implantation of the organized tissue into the organism. At least a subset of the cells of the organized tissue comprises a bioactive compound, wherein the organized tissue is produced by mixing the cells with an extracellular matrix to create a suspension and further mixing the cells with at least one vasculogenic factor. The bioactive compound is produced sufficiently to provide a therapeutic effect to the organism upon implantation of the organized tissue into the organism. The implanted organized tissue is vascularized.

The invention also provides a method of providing a bioactive compound to an organism in therapeutic need thereof comprising implanting into the organism an organized tissue having a three-dimensional geometry approximating the in vivo gross morphology of a tissue of interest that is retained upon implantation of the organized tissue into the organism. At least a subset of the cells of the organized tissue comprises a bioactive compound. At least one vasculogenic factor is added to the organism after the implantation, and the bioactive compound is produced sufficiently to provide a therapeutic effect to the organism upon implantation of the organized tissue into the organism. The implanted organized tissue is vascularized.

The invention also provides for an in vitro method for producing an organized tissue which has an in vivo-like gross and cellular morphology and is vascularized following implantation into an organism, comprising the following steps. Cells of the tissue, wherein at least a subset of the cells comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor are provided. The cells are mixed with an extracellular matrix to create a suspension. The suspension is placed in a vessel having a three-dimensional geometry approximating the in vivo gross morphology of a tissue of interest, wherein the vessel has attachment surfaces. The suspension is allowed to coalesce. The coalesced suspension is cultured under conditions in which the cells connect to the attachment surfaces and form an organized tissue having a three dimensional structure that is retained upon retrieval of the organized tissue. At least a subset of the cells of the organized tissue comprise said DNA sequence encoding the vasculogenic factor.

In one embodiment, the organized tissue further comprises a subset of cells comprising a bioactive compound.

In another embodiment, the organized tissue is comprised of substantially post-mitotic cells.

In another embodiment, an organized tissue has an in vivo-like gross and cellular morphology of the tissue of interest.

In another embodiment, the step of providing comprises isolating primary cells of at least one of the cell types comprising the tissue of interest.

In another embodiment, the step of providing comprises utilizing immortalized cells of at least one of the cell types comprising the tissue.

In another embodiment, prior to the step of providing, a foreign DNA sequence operably linked to a promoter and encoding a protein is introduced to at least a subset of the cells.

In another embodiment, the cells comprise skeletal muscle cells.

In another embodiment, the coalesced suspension exerts a force on said cells parallel to a dimension of said vessel.

In another embodiment, the cells are aligned parallel to a dimension of the vessel.

In another embodiment, the attachment surfaces are positioned at opposite ends of the vessel.

In another embodiment, the organized tissue produces the protein.

The invention also provides an in vitro method for producing an organized tissue which has an in vivo-like gross and cellular morphology and is vascularized following implantation into an organism, comprising the following steps. Cells of the tissue are provided, mixed with an extracellular matrix to create a suspension and further mixed with at least one vasculogenic factor. The suspension is placed in a vessel having a three-dimensional geometry approximating the in vivo gross morphology of a tissue of interest, and having attachment surfaces. The suspension is allowed to coalesce and the coalesced suspension is cultured under conditions in which the cells connect to the attachment surfaces and form an organized tissue having a three dimensional structure that is retained upon retrieval of the organized tissue.

In one embodiment, the organized tissue further comprises a subset of cells comprising a bioactive compound.

The invention also provides an in vitro method for producing an organized tissue which has an in vivo-like gross and cellular morphology and is vascularized following implantation into an organism, comprising the following steps. Cells of the tissue are provided, and are mixed with an extracellular matrix to create a suspension. The suspension is placed in a vessel having a three-dimensional geometry approximating the in vivo gross morphology of a tissue of interest, and having attachment surfaces. The suspension tis allowed to coalesce. The coalesced suspension is cultured under conditions in which the cells connect to the attachment surfaces and form an organized tissue having a three-dimensional structure that is retained upon retrieval of the organized tissue. At least one vasculogenic factor is added to the organism following the implantation.

In one embodiment, the organized tissue further comprises a set of cells comprising a bioactive compound.

The invention also provides for an organized tissue having an in vivo gross cellular morphology and producing a protein of a type or produced in an amount not produced normally by a tissue of interest, produced according to the methods described herein.

The invention also provides for an organized tissue producing a bioactive compound of a type or produced in an amount not produced normally by a tissue of interest, where the organized tissue is produced by the steps of: mixing a plurality of cells with an extracellular matrix to create a suspension, wherein at least a subset of the cells comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor and further comprises a bioactive compound. The suspension is placed in a vessel having a three-dimensional geometry approximating the in vivo gross morphology of the tissue of interest, and having attachment surfaces. The suspension is allowed to coalesce; and the coalesced suspension is cultured under conditions in which the cells connect to the attachment surfaces and form an organized tissue having a three-dimensional structure that is retained upon retrieval of the organized tissue from the organism. The organized tissue is vascularized following implantation into the organism. The bioactive compound is produced at detectable levels in the tissue.

In one embodiment, the organized tissue, further comprising substantially post-mitotic cells.

In another embodiment, the organized tissue comprises an in vivo-like gross and cellular morphology of the tissue of interest.

In another embodiment, the tissue is skeletal muscle.

The invention also provides for an organized tissue producing a bioactive compound of a type or produced in an amount not produced normally by a tissue of interest, where the organized tissue is produced by the following steps. A plurality of cells are mixed with an extracellular matrix to create a suspension, and further mixed with at least one vasculogenic factor. At least a subset of the cells comprises a bioactive compound. The suspension is placed in a vessel having a three-dimensional geometry approximating the in vivo gross morphology of the tissue of interest, and having attachment surfaces. The suspension is allowed to coalesce. The coalesced suspension is cultured under conditions in which the cells connect to the attachment surfaces and form an organized tissue having a three dimensional structure that is retained upon retrieval of the organized tissue from the organism. The organized tissue is vascularized following implantation into said organism. The bioactive compound is produced at detectable levels in the tissue.

The invention also provides for an organized tissue producing a bioactive compound of a type or produced in an amount not produced normally by a tissue of interest, where the organized tissue is produced by the steps of: mixing a plurality of cells with an extracellular matrix to create a suspension, wherein at least a subset of the cells comprises a bioactive compound. The suspension is placed in a vessel having a three-dimensional geometry approximating the in vivo gross morphology of the tissue of interest, and having attachment surfaces. The suspension is allowed to coalesce and is cultured under conditions in which the cells connect to the attachment surfaces and form an organized tissue having a three dimensional structure that is retained upon retrieval of the organized tissue from the organism. The organized tissue is implanted into the organism and at least one vasculogenic factor is added to theorgansim. The organized tissue is vascularized following implantation into the organism. The bioactive compound is produced at detectable levels in the tissue.

The invention also provides for an organized tissue having an in vivo-like gross and cellular morphology of a tissue of interest and producing a bioactive compound of a type or produced in an amount not produced normally by the tissue of interest comprising a plurality of cells, wherein at least a subset of the cells comprise a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor, and further comprising a bioactive compound. The cells form an organized tissue that has a three-dimensional structure that is retained upon retrieval of the organized tissue from the organism. The organized tissue is vascularized following implantation into the organism. The bioactive compound is produced at detectable levels in the tissue.

In one embodiment, the organized tissue comprises substantially post-mitotic cells.

In another embodiment, the organized tissue approximates the in vivo gross morphology of the tissue of interest.

The invention also provides for an organized tissue having an in vivo-like gross and cellular morphology of a tissue of interest and producing a bioactive compound of a type or produced in an amount not produced normally by the tissue of interest comprising: a plurality of cells, wherein at least a subset of the cells comprises a bioactive compound, wherein the cells form an organized tissue having a three-dimensional structure that is retained upon retrieval of the organized tissue from the organism, wherein the organized tissue is formed by mixing the cells with an extracellular matrix to create a suspension, and further mixing the cells with at least one vasculogenic factor. The organized tissue is vascularized following implantation into an organism and the bioactive compound is produced at detectable levels in the tissue.

The invention also provides for an organized tissue having an in vivo-like gross and cellular morphology of a tissue of interest and producing a bioactive compound of a type or produced in an amount not produced normally by said tissue of interest comprising a plurality of cells, wherein at least a subset of the cells comprises a bioactive compound, wherein the cells form an organized tissue having a three-dimensional structure that is retained upon retrieval of the organized tissue from said organism, wherein at least one vasculogenic factor is added to the organism following implantation, wherein the organized tissue is vascularized following implantation into an organism; and; wherein the bioactive compound is produced at detectable levels in the tissue.

The invention also provides for an organized tissue producing a protein produced by the following steps. A plurality of mammalian cells, wherein at least a subset of the cells comprises a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor and further comprises a bioactive compound are provided. The cells are mixed with an extracellular matrix to create a suspension and the suspension is placed in a vessel having a three-dimensional geometry approximating the in vivo gross morphology of a tissue of interest, and having attachment surfaces. The suspension is allowed to coalesce; and is cultured under conditions in which the cells connect to the attachment surfaces, wherein the suspension of cells forms an organized tissue that has a three-dimensional structure that is retained upon implantation of the tissue into a mammal. The tissue is vascularized upon implantation into an organism. The bioactive compound is produced sufficiently to provide a therapeutic effect to the organism once the organized tissue is implanted into the organism.

In one embodiment, the organized tissue further comprises substantially post-mitotic cells.

The invention also provides for an organized tissue producing a protein produced by the steps of mixing a plurality of mammalian cells, wherein at least a subset of the cells comprises a bioactive compound, wherein the cells are mixed with an extracellular matrix to create a suspension and further mixed with at least one vasculogenic factor. The suspension is placed in a vessel having a three-dimensional geometry approximating the in vivo gross morphology of a tissue of interest, and having attachment surfaces. The suspension is allowed to coalesce; and is cultured under conditions in which the cells connect to the attachment surfaces, wherein the suspension of cells forms an organized tissue that has a three-dimensional structure that is retained upon implantation of the tissue into a mammal, and wherein the tissue is vascularized upon implantation into an organism. The bioactive compound is produced sufficiently to provide a therapeutic effect to the organism once the organized tissue is implanted into the organism.

The invention also provides for an organized tissue producing a protein produced by the steps of: mixing a plurality of mammalian cells, wherein at least a subset of the cells comprises a bioactive compound, wherein the cells are mixed with an extracellular matrix to create a suspension. The suspension is placed in a vessel having a three-dimensional geometry approximating the in vivo gross morphology of a tissue of interest, and having attachment surfaces. The suspension is allowed to coalesce; and is cultured under conditions in which the cells connect to the attachment surfaces. The suspension of cells forms an organized tissue that has a three-dimensional structure that is retained upon implantation of the tissue into a mammal. The organized tissue is implanted into the organism and at least one vasculogenic factor is added to the organism. The tissue is vascularized upon implantation into the organism. The bioactive compound is produced sufficiently to provide a therapeutic effect to the organism once the organized tissue is implanted into the organism.

The invention also provides for an organized tissue having a three-dimensional cellular organization of a tissue of interest that is retained upon implantation of the tissue into an organism, the tissue producing a bioactive compound of a type or in an amount not normally produced by a tissue of interest, comprising a plurality of cells, wherein at least a subset of the cells comprises a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor and further comprising a bioactive compound. The organized tissue has a three-dimensional structure that is retained upon retrieval of the tissue from the organism, and the organized tissue is vascularized following implantation into the organism. The bioactive compound is produced to detectable levels in the tissue of interest.

In one embodiment, the organized tissue comprises substantially post-mitotic cells.

In another embodiment, the organized tissue has a three-dimensional geometry approximating the in vivo gross morphology of the tissue of interest.

The invention also provides for an organized tissue having a three-dimensional cellular organization of a tissue of interest that is retained upon implantation of the tissue into an organism, the tissue producing a bioactive compound of a type or in an amount not normally produced by a tissue of interest, comprising a plurality of cells, wherein at least a subset of the cells comprises a bioactive compound. The organized tissue is formed by mixing the cells with an extracellular matrix to create a suspsension and further mixing with at least one vasculogenic factor. The tissue has a three-dimensional geometry that is retained upon retrieval of the tissue from the organism. The organized tissue is vascularized following implantation into the organism. The bioactive compound is produced to detectable levels in the tissue of interest.

The invention also provides for an organized tissue having a three-dimensional cellular organization of a tissue of interest that is retained upon implantation of the tissue into an organism, the tissue producing a bioactive compound of a type or in an amount not normally produced by a tissue of interest, comprising a plurality of cells, wherein at least a subset of the cells comprises a bioactive compound. The tissue has a three-dimensional geometry that is retained upon retrieval of the tissue from the organism. The organized tissue is implanted into the organism and at least one vasculogenic factor is added to the organism following implantation. The organized tissue is vascularized following implantation into the organism; and the bioactive compound is produced to detectable levels in the tissue of interest.

The invention also provides an organized tissue attached to a surface of a substrate, the tissue producing a bioactive compound, comprising a plurality of cells, wherein at least a subset of the cells comprise a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor and further comprising a bioactive compound. The cells form an organized tissue having a three-dimensional geometry that is retained upon retrieval of the organized tissue from an organism into which it has been implanted, and the organized tissue is attached to the surface of a substrate. The organized tissue is vascularized upon implantation into an organism, and the bioactive compound is produced to detectable levels in the tissue of interest.

In one embodiment, the organized tissue comprises substantially post-mitotic cells.

In another embodiment, the organized tissue comprises an in vivo gross morphology of said tissue of interest.

In another embodiment, the substrate being selected from the group consisting of metal or plastic.

In another embodiment, the metal substrate is steel mesh having a longitudinal axis and first and second points for attachment, and wherein the first and second attachment sites of the tissue are atached, respectively, to the first and second points of attachment.

The invention also provides for an organized tissue attached to a surface of a substrate, the tissue producing a bioactive compound, comprising a plurality of cells, wherein at least a subset of the cells comprises a bioactive compound, wherein the organized tissue is formed by mixing the cells with an extracellular matrix to create a suspension and further mixing with at least one vasculogenic factor, wherein the cells form an organized tissue having a three-dimensional geometry approximating the in vivo gross morphology of a tissue of interest and that is retained upon retrieval of the organized tissue from an organism into which it has been implanted, and wherein the organized tissue is attached to the surface of a substrate. The organized tissue is vascularized upon implantation into an organism and the bioactive compound is produced to detectable levels in the tissue of interest.

The invention also provides for an organized tissue attached to a surface of a substrate, the tissue producing a bioactive compound, comprising a plurality of cells, wherein at least a subset of the cells comprises a bioactive compound. The cells form an organized tissue having a three-dimensional geometry approximating the in vivo gross morphology of a tissue of interest and that is retained upon retrieval of the organized tissue from an organism into which it has been implanted, and the organized tissue is attached to the surface of a substrate. The organized tissue is implanted into said organism and at least one vasculogenic factor is added to said organism following implantation. The organized tissue is vascularized upon implantation into an organism, and; the bioactive compound is produced to detectable levels in the tissue of interest.

The invention also provides for a method of delivering a vasculogenic factor to an organism comprising the steps of: growing in vitro a plurality of cells wherein at least a subset of cells comprises a DNA sequence encoding a vasculogenic factor or a DNA sequence encoding a vasculogenic factor operably linked to a promoter. The cells are mixed with an extracellular matrix to create a suspension. The suspension is placed in a vessel having a three-dimensional geometry approximating the in vivo gross morphology of a tissue of interest, and having attachment surfaces. The suspension is allowed to coalesce and is cultured under conditions in which said cells connect to said attachment surfaces and form an organized tissue having an in vivo-like gross and cellular morphology that is retained upon retrieval of said organized tissue. The organized tissue is implanted into the organism, wherein the organized tissue becomes vascularized; and whereby the vasculogenic factor is produced and delivered to the organism. The vasculogenic factor is of a type or produced in an amount not produced by the tissue of interest, and is produced sufficiently to provide a therapeutic effect to the organism upon implantation of theorganized tissue into the organism.

The invention also provides for a method of delivering a vasculogenic factor to an organism comprising the steps of: growing in vitro a plurality of cells,wherein at least a subset of cells comprises a DNA sequence encoding a vasculogenic factor, or a DNA sequence encoding a vasculogenic factor operably linked to a promoter, and wherein said cells are mixed with an extracellular matrix to create a suspension. The suspension is placed in a vessel wherein the cells form an organized tissue of interest having a three dimensional cellular organization which is retained upon implantation into the organism. The organized tissue is implanted into the organism, whereby the organized tissue is vascularized; and wherein the vasculogenic factor is produced and delivered to the organism sufficiently to provide a therapeutic effect to the organism. The vasculogenic factor is of a type or produced in an amount not produced by the tissue of interest.

The invention also provides for a method of providing a vasculogenic factor to an organism in therapeutic need thereof comprising: implanting into the organism an organized tissue having a three-dimensional geometry that is retained upon retrieval of said organized tissue. At least a subset of cells comprise a DNA sequence encoding a vasculogenic factor or a DNA sequence encoding a vasculogenic factor operably linked to a promoter, and at least a subset of cells of the organized tissue comprises a vasculogenic factor to be delivered to said organism. The vasculogenic factor is produced sufficiently to provide a therapeutic effect to the organism upon implantation of the organized tissue into the organism. The implanted organized tissue is vascularized.

The invention also provides for a method of providing a vasculogenic factor to an organism in therapeutic need thereof comprising implanting into the organism an organized tissue having a three-dimensional geometry approximating the in vivo gross morphology of a tissue of interest that is retained upon implantation of said organized tissue into said organism. At least a subset of the cells of the organized tissue comprises a DNA sequence encoding a vasculogenic factor or a DNA sequence encoding a vasculogenic factor operably linked to a promoter. The vasculogenic factor is produced sufficiently to provide a therapeutic effect to the organism upon implantation of the organized tissue into the organism. The implanted organized tissue is vascularized.

The invention also provides for an organized tissue producing a vasculogenic factor of a type or produced in an amount not produced normally by a tissue of interest, where the organized tissue is produced by the steps of mixing a plurality of cells with an extracellular matrix to create a suspension, wherein at least a subset of the cells comprises a DNA sequence encoding a vasculogenic factor or a DNA sequence encoding a vasculogenic factor operably linked to a promoter. The suspension is placed in a vessel having a three-dimensional geometry approximating the in vivo gross morphology of the tissue of interest, and having attachment surfaces. The suspension is allowed to coalesce; and is cultured under conditions in which the cells connect to the attachment surfaces and form an organized tissue having a three-dimensional structure that is retained upon retrieval of the organized tissue from the organism. The organized tissue is vascularized following implantation into the organism; and the vasculogenic factor is produced at detectable levels in the tissue.

The invention also provides for an organized tissue having an in vivo-like gross and cellular morphology of a tissue of interest and producing a vasculogenic factor of a type or produced in an amount not produced normally by said tissue of interest comprising a plurality of cells, wherein at least a subset of the cells comprise a DNA sequence encoding a vasculogenic factor or a DNA sequence encoding a vasculogenic factor that is operably linked to a promoter. The cells form an organized tissue has a three-dimensional structure that is retained upon retrieval of the organized tissue from the organism. The organized tissue is vascularized following implantation into an organism; and wherein the vasculogenic factor is produced at detectable levels in the tissue.

The invention also provides for an organized tissue producing a vasculogenic factor produced by the steps of: mixing a plurality of mammalian cells, wherein at least a subset of the cells comprises a DNA sequence encoding a vasculogenic factor, or a DNA sequence encoding a vasculogenic factor that is operably linked to a promoter, wherein the cells are mixed with an extracellular matrix to create a suspension; placing the suspension in a vessel having a three-dimensional geometry approximating the in vivo gross morphology of a tissue of interest, and having attachment surfaces; allowing the suspension to coalesce; and culturing the coalesced suspension under conditions in which the cells connect to the attachment surfaces. The suspension of cells forms an organized tissue that has a three-dimensional structure that is retained upon implantation of the tissue into a mammal, and the tissue is vascularized upon implantation into an organism. The vasculogenic factor is produced sufficiently to provide a therapeutic effect to said organism once the organized tissue is implanted into the organism.

The invention also provides for an organized tissue having a-three-dimensional cellular organization of a tissue of interest that is retained upon implantation of the tissue into an organism, the tissue producing a vasculogenic factor of a type or in an amount not normally produced by a tissue of interest, comprising a plurality of cells, wherein at least a subset of the cells comprises a DNA sequence encoding a vasculogenic factor, or a DNA sequence encoding a vasculogenic factor that is operably linked to a promoter. The organized tissue has a three-dimensional structure that is retained upon retrieval of said tissue from said organism. The organized tissue is vascularized following implantation into said organism; and the vasculogenic factor is produced to detectable levels in said tissue of interest.

The invention provides a number of advantages. For example, implantation of an organized tissue produced in vitro, wherein the organized tissue becomes vascularized following implantation provides quantifiable, reproducible, and localized and systemic delivery of bioactive compounds to an organism. Prior to implantation, the production of bioactive compounds by the organized tissue may be measured and quantified per unit time, per unit mass, or relative to any other physiologically-relevant parameter. In addition, the capability of an organized tissue to sustain production of bioactive compounds can be assessed by culturing for extended periods and assaying of compound production with time. Further, an organized tissue that becomes vascularized following implantation can be used to deliver bioactive compounds, for example proteins, into the systemic circulation. The organized tissue of the invention therefore provides for a delivery vehicle for introducing large, and/or unstable proteins (for example Factor VIII) directly into the bloodstream.

Moreover, because the organized tissue is implanted at a defined anatomical location as a discrete collection of cells, it may be distinguished from host tissues, removed post-implantation from the organism, and reimplanted into the organism at the same or a different location at the time of removal or following an interim period of culturing in vitro. This feature facilitates transient, localized or systemic delivery of the bioactive compound. Restriction of the cells producing bioactive compounds to particular anatomical sites may enhance the controlled delivery of bioactive compounds, especially where the organized tissue functions as a paracrine organ. The efficiency of delivery of a bioactive compound (i.e., the amount of the bioactive compound delivered to obtain a desired serum concentration) is also enhanced as compared to direct subcutaneous injection. Likewise, the efficiency of implanting post-mitotic cells containing a bioactive compound and/or a DNA sequence encoding a vasculogenic factor or a factor that increases the expression of a vasculogenic factor into an organism (i.e., the number of cells in a post-mitotic state as a percentage of the initial number of cells containing a bioactive compound and/or a DNA sequence encoding a vasculogenic factor or a factor that increases the expression of a vasculogenic factor) is enhanced by organoid implantation as compared to the implantation of individual mitotic cells. For example, skeletal muscle organoids produced in vitro include post-mitotic myofibers representing up to 70% of the initial myoblasts containing a bioactive compound and/or a DNA sequence encoding a vasculogenic factor, or a factor that increases the expression of a vasculogenic factor whereas direct implantation of the myoblasts results in post-mitotic myofibers representing less than 10% of the initial cells.

In addition, because substantially all of the implanted cells are at least partially differentiated, migration of these cells to other anatomical sites is reduced. Moreover, implantation of post-mitotic, non-migratory myofibers containing a bioactive compound and/or a vasculogenic factor or a factor that increases the expression of a vasculogenic factor reduces the possibility of cell transformation and tumor formation. The implantation of an organized tissue may even enhance the functional and structural characteristics of the host tissue.

Furthermore, because the method of producing a tissue having an in vivo-like gross and cellular morphology may be achieved without the application of external forces by mechanical devices, the apparatus for producing such a tissue is readily adaptable to standard cell and tissue culture systems. The apparatus and method may also be used to produce bone, cartilage, tendon, and cardiac tissues as these tissues include cell types which organize in response to external forces. In addition, the apparatus includes widely available, easily assembled and relatively inexpensive components.

Other advantages and features of the invention will be apparent from the detailed description, and from the claims.

### BRIEF DESCRIPTION OF THE DRAWINGS

FIG. 1 is graph demonstrating that rhVEGF secreted by rhVEGF BAMs is biologically active.

FIG. 2 demonstrates the survival of postmitotic myofibers in subcutaneously implanted BAMs.

FIG. 3 is a graph demonstrating mVEGF levels in implanted rmVEGF or control BAMs.

FIG. 4 demonstrates an increase in angiogenesis in subcutaneously implanted rmVEGF BAMs.

FIG. 5 is a time course of angiogenesis in implanted BAMs.

FIG. 6 is a time course of capillary density in ischemic tibialis of mice receiving rmVEGF.

FIG. 7 is a graph demonstrating the predictability of rmVEGF BAM stimulation of capillary growth in vivo from preimplantation in vitro secretion levels.

FIG. 8 is a diagram of a vessel for growing skeletal muscle tissues which will have an in vivo like gross and cellular morphology.

FIG. 9 is a schematic perspective view of a sleeve of the present invention with organized tissue anchored therein.

FIG. 10 is a schematic perspective view of an alternative embodiment of the sleeved organized tissue of FIG. 9, showing a sleeve having two organized tissues anchored therein.

FIG. 11 is a schematic plan view of an alternative embodiment of the present invention, showing organized tissue anchored to a tension maintaining member.

### DETAILED DESCRIPTION

The invention provides an organized tissue that is vascularized following implantation into an organism. The organized tissue of the invention can be used for delivery of a vasculogenic factor and/or a bioactive compound of interest. The organized tissue of the invention can also be used for treating disease. The invention provides for an organized tissue that becomes vascularized following implantation and can deliver proteins into the systemic circulation (directly into the bloodstream), unlike delivery methods such as gene therapy, encapsulated cell methods, drug delivery microcapsule or reservoir approaches, protein injection approaches, or methods wherein cells are placed onto pre-vascularized beds. The organized tissue of the invention therefore provides for a delivery vehicle for introducing large, and/or unstable proteins (for example Factor VIII) directly into the bloodstream.

Definitions

As used herein, “vascularization” means an in growth of blood vessels or an incorporation of blood vessels, for example into an organized tissue of the invention. “Vascularization” refers to “self-vascularization” wherein the organized tissue of the invention provides the stimulus for the vascularization. “Vascularization” also refers to an in growth of blood vessels into the tissue adjacent to the site of implantation of the organized tissue. “Vascularization” refers to the detection of blood vessels, for example, in an organized tissue of the invention or in tissue adjacent to or surrounding the site of implantation of the organized tissue. “Vascularization” also refers to an increain (2-fold, 5-fold, 10-fold or more) in the amount of detectable vlood vessels. In certain embodiments, vascularization occurs within 10cm from the site of implantation. Vascularization is detected by assaying for blood vessel density by measuring the number or proliferation of endothelial cells. Preferably, a vascularized implanted organized tissue of the invention means an organized tissue wherein at least 1%, preferably 5-20% and most preferably 30% or more of the total cross-sectional area of the organized tissue demonstrates positive staining for an endothelial cell marker, for example CD31 or vonWillebrand Factor. Alternatively, a vascularized implanted organized tissue of the invention means an organized tissue wherein at least 1%, preferably 5-20% and most preferably 30% or more of the total cross-sectional area of the organized tissue demonstrates positive staining for artery specific marker, ephrinB2 (Mukouyama et al., 2002, Cell, 109:693-705). “Vascularization” is also measured by 1) assaying for blood flow, for example with doppler, MRI or ultrasound, 2) imaging for endothelial cells or smooth muscle cells with histology, microscopy, microsphere beads or immunohistochemistry, 3) by evaluating skin color, or 4) by treadmill testing (Tsurumi et al., 1997, Circulation, 96:382-8).

As used herein, “angiogenesis” refers to the formation of new blood vessels, mainly capillaries.

As used herein, “vasculogenesis” refers to the conversion of capillaries into arterioles (for example as described in Yancopoulos et al., 1998, Cell, 93:661-664). “Vasculogenesis” also refers to the formation of blood vessels during initial tissue development.

“Arteriogensis” refers to the transformation of capillaries or veins into vessels with a smooth muscle cell layer surrounding endothelial cells.

As used herein, “blood vessels” means vessels that carry blood to and from all parts of the body, including arteries, veins, arterioles and capillaries.

As used herein, “vascularized following implantation” means that after an organized tissue of the invention is implanted into an organism, there is an in growth of blood vessels into the organized tissue. This vascularization can be observed at anytime and can be detected using immunocytochemical stains known in the art and described herein, following implantation and is maintained for at least 4 weeks or longer following implantation. The invention provides for an organized tissue that becomes vascularized, wherein the vascular bed remains intact for as long as the organized tissue implant produces a detectable level of a bioactive molecule.

As used herein, “vasculogenic factor” refers to any molecule (for example a protein), that stimulates formation of blood vessels or increases the number and density of existing blood vessels. A “vasculogenic factor” includes a vasculogenic factor. A “vasculogenic factor” can be endogenous or exogenous to the cell or cells comprising the organized tissue that comprise a DNA sequence encoding the vasculogenic factor or that are mixed with a vasculogenic factor prior to implantation or are treated with a vasculogenic factor after implantation. Preferably, a “vasculogenic factor” stimulates the formation of blood vessels to a detectable level. Blood vessel formation is detected as described above. Alternatively, a “vasculogenic factor” increases the number or density of blood vessels by at least 2-fold or more. Alternatively, a “vasculogenic factor” increases (at least 2-fold, preferably 5-fold and more preferably 10-fold or more) the rate of vascularization as compared to the rate of vascularization in the absence of an angiogenic factor. “Vasculogenic factors” useful according to the invention include but are not limited to VEGF A, B, C, D, E (Vascular endothelial growth factor), FGF 1, 2, 3, 4, 5 (Fibroblast growth factor), PDGF AA, BB, AB (platelet derived growth factor), angiopoeitins, MCP (macrophage chemoattractant protein), EPO (erythropoeitin), and IL 1-22 (interleukins)., and all of the vasculogenic factors included in Table I.

TABLE I

| “Vasculogenic | | Genbank | Genbank |
|---|---|---|---|
| Factor” | Genbank Accession # | Accession # | Accession # |
| VEGF A | XM_166457 | NM_003376 | AF37895 |
| VEGF B | NM_003377 | | |
| VEGF C | NM_005429 | | |
| VEGF D | BC027948 | | |
| VEGF E | AF106020(retrovector) | | |
| FGF-1 | BC032697 | | |
| FGF-2 | P09038 | | |
| FGF-3 | P11487 | | |
| FGF-4 | P08620 | | |
| FGF-5 | P12034 | | |
| FGF-6 | P10767 | | |
| FGF-7 | P21781 | | |
| FGF-12 | Q92912 | | |
| PDGF-A | P04085 | | |
| PDGF-B | P01127 | | |
| MCP-1 | S71513.1 | | |
| EPO | AH009005 | M11319 | |
| IL-1 | M28983 | X03833 | |
| IL-2 | S77834 | | |
| IL-3 | M14743 | | |
| IL-4 | M13982 | | |
| IL-5 | J03478 | | |
| IL-6 | AF372214 | | |
| IL-7 | M29696 | AH006906 | |
| IL-8 | M28130 | | |
| IL-9 | AF361105 | | |
| IL-10 | AF418271 | | |
| IL-11 | M81890 | M57765 | |
| IL-12 p35 | AF101062 | | |
| IL-12 p40 | AF180563 | | |
| IL-13 | L06801 | | |
| IL-14 | L15344 | | |
| IL-15 | U14407 | | |
| IL-16 | AF053412 | | |
| IL-17 | NM_002190 | | |
| IL-18 | AY044641 | | |
| IL-19 | AF276915 | | |
| IL-20 | AF402002 | | |
| IL-21 | NM_021803 | | |
| IL-22 | NM_020525 | | |
| IL-23 | AF301620 | | |
| IL-24 | NM_006850 | | |
| IL-25 | AF458059 | | |
| ANG-1 | XM_114636 | | |
| ANG-2 | XM_034835 | | |
| ANG-3 | AF074332 | | |
| ANG-4 | NM_015985 | | |

A “vasculogenic factor” includes a single protein or a combination of two or more proteins . For example, the invention provides for two or more “vasculogenic factors” wherein each vasculogenic factor is delivered with different kinetics. This can be accomplished by preparing an organized tissue comprising n copies of a gene encoding a first vasculogenic factor and greater than n copies of a gene encoding a second vasculogenic factor etc . . . This is also accomplished by providing vasculogenic factors that are either stably present and expressed (for example in a retrovirus AAV vector) or transiently expressed and present (in an adenovirus or non-viral vector). This is also accomplished by implanting organized tissues comprising autologous cells expressing a vasculogenic factor, such that the organized tissue will be chronic or long-lived or comprising allogeneic cells expressing a vasculogenic factor, such that the organized tissue will be acutely present or will be sub-chronically reasorbed.

As used herein, “endogenous” means naturally present in, native, originating from or due to influences from inside of, for example, an organism or a cell.

As used herein, “exogenous” means not naturally present, foreign, originating from or due to influences from outside of, for example, an organism or a cell.

As used herein, “allogeneic” means derived from a genetically matched relative of the patient or by an unrelated (but genetically similar) donor.

As used herein, “autologous” means derived from the organism into which the organized tissue is implanted.

As used herein, a “factor that increases the expression of a vasculogenic factor” refers to a factor that increases the expression of a vasculogenic factor by at least 2-fold and includes but is not limited to HIF-1 alpha (Genbank Accession Nos: AB073325, AF003695, U22431) and hypoxia inducible factor (Genbank Accession Nos: BC026139, AF335324, AB073325).

As used herein, “DNA sequence encoding” refers to a DNA polynucleotide, either in its native state or in a recombinant form, that can be transcribed and/or translated to produce the mRNA for and/or a polypeptide or a fragment thereof, for example a vasculogenic factor as described herein. A DNA sequence encoding, for example, a vasculogenic factor, useful according to the invention, includes an endogenous or an exogenous sequence. A “DNA sequence encoding”, for example, a vasculogenic factor, can be present on a vector, either alone or in combination with one or more DNA sequences encoding additional vasculogenic factors and/or one or more bioactive compounds useful according to the invention. A “DNA sequence encoding” for example a vasculogenic factor can be operably linked to a promoter.

As used herein, a “promoter” refers to a region of DNA involved in binding of RNA polymerase to initiate transcription. A “promoter” useful according to the invention includes a promoter that is either endogenous or exogenous to the DNA sequence to which it is operably linked. The invention also provides for regulatable promoters, for example, promoters that are activated by a particular factor, for example, a factor that increases the expression of a vasculogenic factor, or a promoter that is activated under certain environmental conditions, for example, under ischemic conditions, for example the HIF-1 alpha promoter.

As used herein, the term “operably linked” refers to the respective coding sequence being fused in-frame to a promoter, enhancer, termination sequence, and the like, so that the coding sequence is faithfully transcribed, spliced, and translated, and the other structural features are able to perform their respective functions.

The term “vector” or “expression vector” refers to a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. In the present specification, “plasmid” and “vector” are sometimes used interchangeably as the plasmid is the most commonly used form of vector at present. However, the invention is intended to include such other forms of expression vectors which serve equivalent functions and which are, or become, known in the art.

As used herein, “implantation” refers to the introduction of an organized tissue of the invention into any site of an organism. An organized tissue of the invention can be implanted into any tissue of interest., as described below in the section entitled, “Implantation”.

As used herein, by a “bioactive compound” is meant a compound which influences the biological structure, function, or activity of a cell or tissue of a living organism. A bioactive compound includes a nucleic acid encoding a bioactive compound. In certain embodiments of the invention, the nucleic acid sequence encoding a bioactive compound is operably linked to a promoter, as described herein. The invention provides for inducible or regulatable promoters, or constitutively active promoters. Inducible” refers to expressed in the presence of an exogenous or endogenous chemical (for example an alcohol, a hormone, or a growth factor), in the presence of light and/or in response to developmental changes or a particular physiological condition, for example ischemia. A “constitutively active promoter” is expressed in the presence or absence of exogenous or endogenous chemicals. That is, a “constitutive” promoter is continuously active and is not regulatable. The invention also provides for a nucleic acid sequence encoding a bioactive compound that is operably linked to a promoter that is either endogenous or exogenous to the nucleic acid sequence. A bioactive compound includes a nucleic acid, including functional, non-coding RNA for example inhibitory RNA or ribozymes, or a protein, for example Factor VIII. (Genbank Accession No: AAH22513). A “bioactive compound” of the invention includes proteins, particularly large and/or unstable proteins or compounds. A “bioactive compound” includes but is not limited to cytokines, growth factors, hormones, interleukins, antibodies, antibody fragments, viral and non-viral vectors, RNA, DNA, and fusion proteins.

As used herein, “unstable” refers to a protein with a half-life that is less than one hour, for example interferons or interleukins.

As used herein, “large” refers to a protein that is 100,000 kD or larger including, for example, Factor VIII which is 160,000 kD as well as antibody molecules.

As used herein, “organism” refers to any living thing including mammal or avian.

As used herein, “mammal” refers to any mammal including human, mouse, rat, sheep, rabbit, goat, monkey, horse, hamster, pig or cow. A non-human mammal according to the invention is any mammal that is not a human, including but not limited to a mouse, rat, sheep, rabbit, goat, monkey, horse, hamster, pig or a cow.

By “organized tissue” or “organoid” is meant a tissue formed in vitro from a collection of cells having a cellular organization and gross morphology similar to that of the tissue of origin for at least a subset of the cells in the collection. An organized tissue or organoid may include a mixture of different cells, for example, muscle, fibroblast, and nerve cells, but must exhibit the in vivo cellular organization and gross morphology of a tissue including at least one of those cells, for example, the organization and morphology of muscle tissue. The invention provides for an organized tissue that becomes vascularized following implantation and can deliver proteins into the systemic circulation (directly into the bloodstream), unlike delivery methods such as gene therapy, encapsulated cell methods, drug delivery microcapsule or reservoir approaches, protein injection approaches, or methods wherein cells are placed onto pre-vascularized beds, or wherein cells are placed in an avascular bed and off-loaded by diffusion.

The organized tissue of the invention is of a size and shape whereby it can survive initially, in vitro and in vivo, via a diffusion of nutrients into the organized tissue, and is also three-dimensional, such that it can support the formation of a network (for example an intrinsic network) of blood vessels.

An organized tissue of the invention can contain cells or myofibers which synthesize and locally secrete endogenous or exogenous, or a combination thereof, vasculogenic factors which stimulate or increase vascularization around, within and adjacent to the organized tissue.

As used herein, “around” refers to at least 0.5 micrometers and up to 10 cm. As used herein, “adjacent” means next to or in contact with.

By “in vivo-like gross and cellular morphology of a tissue of interest” is meant a three-dimensional shape and cellular organization substantially similar to that of the tissue in vivo. By “substantially similar to that of the tissue in vivo” is meant that the structure is visibly identical or similar to (for example in terms of morphology or the expression of appropriate marker proteins) or functionally similar to the structure (for example produces at least 5% of the amount of a protein produced by the structure prior to implantation, or performs an enzymatic reaction at a level that is at least 5% of the level of reaction performed by the tissue prior to implantation) prior to implantation.

By “retained” is meant maintained. A three-dimensional structure that is “retained” upon retrieval, means a structure that is substantially identical to the structure prior to implantation. Substantially identical to means that the structure is visibly identical or similar to (for example in terms of morphology or the expression of appropriate marker proteins) or functionally similar to the structure (for example produces at least 5% of the amount of a protein produced by the structure prior to implantation, or perfonns an enzymatic reaction at a level that is at least 5% of the level of reaction performned by the tissue prior to implantation) prior to implantation.

By “extracellular matrix components” is meant compounds, whether natural or synthetic compounds, which function as substrates for cell attachment and growth. Examples of extracellular matrix components include, without limitation, collagen, laminin, fibronectin, vitronectin, elastin, glycosaminoglycans, proteoglycans, heparins, and combinations of some or all of these components (e.g., Matrigel.TM., Collaborative Research, Catalog No. 40234).

The invention contemplates an organized tissue that is prepared by mixing the cells with an extracellular matrix and at least one vasculogenic factor. The vasculogenic factor can be added separately or directly to the extracellular matrix prior to the step of mixing the extracellular matrix with the cells. See for example, Rees et al., 1999, Wound Repair Regen., 7:141-147). Regranex (composition and method of use; Johnson and Johnson) may be useful according to the invention.

The invention also contemplates an organized tissue that is prepared and implanted into an organism as described herein. Following the implantation step, at least one vasculogenic factor is added to the organism such that the organized tissue becomes vascularized. By “added to” is meant, added directly to the wound site, or to the tissue adjacent to or surrounding the implantation site of the organized tissue.

By “tissue attachment surfaces” is meant surfaces having a texture, charge or coating to which cells may adhere in vitro. Examples of attachment surfaces include, without limitation, stainless steel wire, VELCRO™, suturing material (degradable or non-degradable), native tendon, covalently modified plastics (e.g., RGD complex), and silicon rubber tubing having a textured surface.

By “substantially post-mitotic cells” is meant an organoid in which at least 50% of the cells containing a DNA sequence and/or a bioactive compound are non-proliferative. Preferably, organoids including substantially post-mitotic cells are those in which at least 80% of the cells containing a DNA sequence and/or a bioactive compound are non-proliferative. More preferably, organoids including substantially post-mitotic cells are those in which at least 90% of the cells containing a DNA sequence and/or a bioactive compound are non-proliferative. Most preferably, organoids including substantially post-mitotic cells are those in which 99% of the cells containing a DNA sequence and/or a bioactive compound are non-proliferative. Cells of an organoid retaining proliferative capacity may include cells of any of the types included in the tissue. For example, in skeletal muscle organoids, the proliferative cells may include muscle stem cells (i.e., satellite cells) and fibroblasts.

As used herein, “plurality of cells” refers to more than one cell.

As used herein, a “primary cell” refers to a cell that has been isolated directly from a living organism and is not immortalized.

As used herein, an “immortalized cell” refers to a cell which can grow and reproduce indefinitely and without restrictions. In certain embodiments, an “immortalized cell” has been transformed.

As used herein, a “therapeutic effect” refers to ameliorating the symptoms of a disease or disorder, by at least 10%, preferably 10-50% and more preferably to undetectable levels.

In Vitro Production of Organized Tissues that are Vascularized Following Implantation

The production of organized tissues useful according to the invention are described in detail in U.S. Pat. No. 5,869,041 and Lu et al., 2001, Circulation, 104:594-599, herein incorporated by reference it their entirety. An organized tissue of the invention includes a bioartificial muscle (BAM).

An organized tissue that is vascularized following implantation into an organism is prepared by producing an organized tissue from a plurality of cells, wherein at least a subpopulation of cells comprises a DNA sequence encoding either an endogenous or exogenous vasculogenic factor. The invention provides for cells wherein the sequence encoding the vasculogenic factor is under the control of a promoter. The invention also provides for an organized tissue that is vascularized following implantation into an organism comprising a plurality of cells, a subset of which comprise a DNA sequence encoding a compound that increases the expression of an endogenous gene encoding a vasculogenic factor. Alternatively, an organized tissue that is vascularized following implantation into an organism is produced from a subpopulation of cells that are mixed with at least one vasculogenic factor. The invention also provides for an organized tissue that is vascularized following implantation, wherein the organized tissue is implanted and at least one vasculogenic factor is administered to the wound site. The organized tissue of the invention can further comprise a subset of cells comprising a bioactive compound.

In Vitro Production of Organized Tissues of the Invention

Organized tissues having in vivo-like gross and cellular morphology may be produced in vitro from the individual cells of a tissue of interest. As a first step in this process, disaggregated or partially disaggregated cells are mixed with a solution of extracellular matrix components to create a suspension. This suspension is then placed in a vessel having a three dimensional geometry which approximates the in vivo gross morphology of the tissue and includes tissue attachment surfaces coupled to the vessel. The cells and extracellular matrix components are then allowed to coalesce or gel within the vessel, and the vessel is placed within a culture chamber and surrounded with media under conditions in which the cells are allowed to form an organized tissue connected to the attachment surfaces.

Although this method is compatible with the in vitro production of a wide variety of tissues, it is particularly suitable for tissues in which at least a subset of the individual cells are exposed to and impacted by mechanical forces during tissue development, remodeling or normal physiologic function. Examples of such tissues include muscle, bone, skin, nerve, tendon, cartilage, connective tissue, endothelial tissue, epithelial tissue, and lung. More specific examples include skeletal, cardiac (i.e., striated), and smooth muscle, stratified or lamellar bone, and hyaline cartilage. This method is also compatible with the in vitro production of adipose tissue, and tissues comprising either mesenchymal stem cells, bone marrow derived cells, bone marrow stromal cells and neural connective tissue. Where the tissue includes a plurality of cell types, the different types of cells may be obtained from the same or different organisms, the same or different donors, and the same or different tissues. Moreover, the cells may be primary cells or immortalized cells. The invention also provides for organized tissues that become vascularized following implantation into an organism comprising either autologous or allogeneic cells, as compared to the organism into which the organized tissue is transplanted. Furthermore, all or some of the cells of the organized tissue may contain a bioactive compound (as described herein).

The composition of the solution of extracellular matrix components will vary according to the tissue produced. Representative extracellular matrix components include, but are not limited to, collagen, laminin, fibronectin, vitronectin, elastin, glycosaminoglycans, proteoglycans, and combinations of some or all of these components (e.g., Matrigel™, Collaborative Research, Catalog No. 40234). In tissues containing cell types which are responsive to mechanical forces, the solution of extracellular matrix components preferably gels or coalesces such that the cells are exposed to forces associated with the internal tension in the gel.

Culture conditions will also vary according to the tissue produced. Methods for culturing cells are well known in the art and are described, for example, in Animal Cell Culture: A Practical Approach, (R. I. Fveshney, ed. IRL Press, 1986). In general, the vessel containing a coalesced suspension of cells and extracellular matrix components is placed in a standard culture chamber (e.g., wells, dishes, or the like), and the chamber is then filled with culture medium until the vessel is submerged. The composition of the culture medium is varied, for example, according to the tissue produced, the necessity of controlling the proliferation or differentiation of some or all of the cells in the tissue, the length of the culture period and the requirement for particular constituents to mediate the production of a particular bioactive compound. The culture vessel may be constructed from a variety of materials in a variety of shapes as described below.

An apparatus for producing a tissue in vitro having an in vivo-like gross and cellular morphology includes a vessel having a three dimensional geometry which approximates the in vivo gross morphology of the tissue. The apparatus also includes tissue attachment surfaces coupled to the vessel. Such a vessel may be constructed from a variety of materials which are compatible with the culturing of cells and tissues (e.g., capable of being sterilized and compatible with a particular solution of extracellular matrix components) and which are formable into three dimensional shapes approximating the in vivo gross morphology of a tissue of interest. The tissue attachment surfaces (e.g., stainless steel mesh, VELCRO™, or the like) are coupled to the vessel and positioned such that as the tissue forms in vitro the cells may adhere to and align between the attachment surfaces. The tissue attachment surfaces may be constructed from a variety of materials which are compatible with the culturing of cells and tissues (e.g., capable of being sterilized, or having an appropriate surface charge, texture, or coating for cell adherence).

The tissue attachment surfaces may be coupled in a variety of ways to an interior or exterior surface of the vessel. Alternatively, the tissue attachment surfaces may be coupled to the culture chamber such that they are positioned adjacent the vessel and accessible by the cells during tissue formation. In addition to serving as points of adherence, in certain tissue types (e.g., muscle), the attachment surfaces allow for the development of tension by the tissue between opposing attachment surfaces. Moreover, where it is desirable to maintain this tension in vivo, the tissue attachment surfaces may be implanted into an organism along with the tissue.

One vessel according to the invention is shown in **FIG. X. This vessel 1, which is suitable for the in vitro production of a skeletal muscle organoid 3, has a substantially semi-cylindrical shape and tissue attachment surfaces 2 coupled to an interior surface of the vessel.

In Vitro Production of Tissues Having In Vivo-like Gross and Cellular Morphology and Producing a Vasculogenic Factor

Cells producing a vasculogenic factor of the invention are produced by methods of transfection or transduction well known in the art., using an appropriate viral or non-viral vector comprising a DNA sequence encoding a vasculogenic factor of interest. In one embodiment, retroviral producer cell lines are generated for a vector producing a vasculogenic factor useful according to the invention. For example, retroviral producer cell lines are generated for LghVEGF₁₆₅SN, along with control vectors LghGHSN, and LgXSN following a two-step transfection/transduction protocol optimized for primary adult mouse myoblasts using E86 ecotropic and PT67 amphotropic packaging cells. Viral-containing medium (vcm) is collected from high titer PT67 clones, and stored at −80° C. pMFG-mVEGF is transfected into Phoenix packaging cells (gift of Dr. Garry Nolan, Stanford University) to generate vcm containing mVEGF retrovirus (or a retrovirus producing any vasculogenic factor useful according to the invention).

Primary mouse myoblasts are isolated from the hind limbs of C3HeB/FeJ 4-6 week old male mice (Jackson Laboratory) and maintained in culture following standard procedures (Powell, C. et al., Gene Therapy Protocols, Humana Press; in press.; Pinset, C. et al., 1996, Cell Biology: A Laboratory Handbook 2nd ed, 1: 226). Isolated cells are transduced with polybrene-supplemented vcm following a centrifugation protocol (Springer, M. et al., 1997, Somat Cell Mol Genet., 23: 203). BAMs for subcutaneous implants are formed from 2×10⁶ transduced myoblasts and are 1 mm×15 mm, (Shansky, J. et al., 1997, In Vitro Cell Dev Biol Anim., 33: 659) while those implanted into ischemic hind limbs are 10 mm long and formed from 1.5×10⁶ myoblasts. BAMs are treated with cytosine arabinoside (1 μg/ml) for 3-6 days before implantation to eliminate proliferating cells as previously described (Vandenburgh, H. et al., 1996, Hum Gene Ther. 7: 2195).

An organized tissue comprising a subpopulation of cells comprising a DNA sequence encoding a vasculogenic factor and further comprising a subpopulation of cells comprising a bioactive compound is prepared as described above, by transducing primary mouse myoblasts (as described above) with polybrene-supplemented virus containing medium from a) retroviral producer cell lines generated for a vector producing a vasculogenic factor of interest and b) retroviral producer cell lines generated for a vector producing a bioactive compound of interest.

The invention provides for the preparation and use of retroviral producer cell lines comprising a vector that expresses both a vasculogenic factor and a bioactive compound of interest. The method described above can also be used to generate an organized tissue comprising a subpopulation of cells comprising a DNA sequence encoding a factor that increases the expression of a vasculogenic factor, either alone, or in combination with a bioactive compound of interest.

An organized tissue that is vascularized following implantation into an organism can be prepared as described above, wherein the organized tissue is formed from cells that do not comprise a DNA sequence encoding a vasculogenic factor or a factor that increases the expression of a vasculogenic factor. Disaggregated or partially disaggregated cells are mixed with a solution of extracellular matrix components to create a suspension and further mixed with at least one vasculogenic factor. A vasculogenic factor can be added at a concentration of pg to mg and can be added in a buffer or gel (for example as in Regranex (Johnson & Johnson). One or more vasculogenic factors can be added. This suspension is then placed in a vessel having a three dimensional geometry which approximates the in vivo gross morphology of the tissue and includes tissue attachment surfaces coupled to the vessel. The cells and extracellular matrix components are then allowed to coalesce or gel within the vessel, and the vessel is placed within a culture chamber and surrounded with media under conditions in which the cells are allowed to form an organized tissue connected to the attachment surfaces.

Alternatively, an organized tissue is prepared as described above, wherein the organized tissue does not comprise a subpopulation of cells comprising a DNA sequence expressing a vasculogenic factor or a factor that increases the expression of a vasculogenic factor. According to this embodiment, the organized tissue is implanted and then at least one vasculogenic factor is added to the organism. A vasculogenic factor according to the invention can be added by injection or as a gel immediately after implantation of the organized tissue and prior to closure of the implantation site.

Sleeved Organized Tissue

The invention also encompasses a sleeved organized tissue (as described in U.S. patent application Ser. No. 20010046488, herein incorporated by reference in its entirety, that can become vascularized.

The invention also encompasses a sleeved organized tissue, wherein the sleeve comprises a biocompatible structure encircling a length of the tissue, or circumferentially surrounding or enclosing the tissue. As used herein, “a length of the tissue” refers to at least 50% of the total length of the tissue, or at least 80%, 90% or even greater than the length of the tissue. Where multiple organized tissues are contained within a sleeve, the sleeve will encompass “a length” of at least one such organized tissue, and possibly also of two, three, four or more plural organized tissues. The sleeved organized tissue according to the invention also includes a sleeved tissue wherein the tissue is substantially encapsulated or surrounded (i e., encircled along a length, where the length of encirclement is at least 50% of the length of the tissue, or 80%, 90%, or fully encapsulated) by the sleeve.

In accordance with another aspect, an in vitro method for producing sleeved organized tissue may be performed, wherein the sleeved organized tissue has a biocompatible structure surrounding the organized tissue in at least one dimension and along a length of the tissue. This method is performed by providing an organized tissue, placing the organized tissue into a sleeve, wherein the sleeve surrounds the organized tissue in at least one dimension and along a length of the tissue.

In accordance with yet another aspect, an in vitro method for producing sleeved organized tissue having a biocompatible structure surrounding the organized tissue in at least one dimension and along a length of the tissue may be performed. This method is performed by providing growing cells, and placing the cells into a sleeve under conditions which permit the cells to form an organized tissue in the sleeve.

As used herein with regard to an organized tissue, the term “substantially encapsulated” refers to that which is surrounded or enclosed by- or contained within a sleeve, either on all sides or on all sides except one or both longitudinal termini, or “points for attachment”. Where a sleeve does not fully cover an end of an organized tissue, the sleeve need not physically coincide in length with the organized tissue, but may extend beyond it for a distance as desired.

As used herein with regard to an organized tissue, the terms “longitudinal terminus” or “point for attachment” refer interchangeably to all or a portion of a face of such a tissue seen when the short aspect of an elongated organized tissue is viewed (i.e., when the long axis of the organized tissue is parallel to the sight line of the viewer). As used herein with regard to a longitudinal terminus or point for attachment, the term “portion” refers to as little as 0.001% of such a terminus or point for attachment.

As used herein, “sleeve” refers to a biocompatible structure, having at least a first point for attachment and a second point for attachment. The sleeve is, in certain preferred embodiments, a porous, preformed structure. The sleeve can have the shape of, for example, a cylinder, a disk, a rectangle, or other suitable geometries The sleeve can also be in the form of a mesh, net, stent or shape-memory material. The sleeve can be constructed from a material selected from the group including, but not limited to, polyacrylates, polymethyl-acrylates, polyalginate, polyvinyl alcohols, polyethylene oxide, polyvinylidene fluoride, polyvinylidenes, polyvinyl chloride, polyurethanes, polyurethane isocyanates, polystyrenes, polyamides, polyaspartate, polyglutamate, cellulose-based polymers, cellulose acetates, cellulose nitrates, polysulfones, polyphosphazenes, polyacrilonitriles, poly(acrilonitrile/covinyl chloride), stretched, woven, extruded or molded polytetrafluoroethylene, stretched, woven, extruded or molded polypropylene, stretched, woven, extruded or molded polyethylene, porous polyvinylidene fluoride, Angel Hair, silicon-oxygen-silicon matrices, polylsine and derivatives, copolymers and mixtures thereof. The sleeve can also be constructed of natural materials including, but not limited to, collagen, extracellular matrix, intestinal mucosa, and metals including, but not limited to, stainless steel, tantalum, titanium and its alloys, and nitinol.

As used herein, “sufficiently flexible” refers to that which is capable of undergoing a change in shape, in particular capable of undergoing expansion or retraction, and capable of conforming to the shape of the organized tissue. As used herein, “flexible” does not refer to that which is capable of undergoing a phase change from a liquid to a solid state.

As used herein, “preformed structure” refers to that which has a predetermined solid shape (e.g., porous tube, mesh, or net) and dimensions thereof prior to the insertion of an organized tissue, or prior to the formation of an organized tissue within such a preformed structure.

As used herein “transplantable, substantially encapsulated, organized tissue” refers to a substantially encapsulated organized tissue capable of being implanted into a host mammal.

As used herein, “porous” or refers to having pores, wherein “pore” refers to a small space by which matter can pass through a membrane As used herein with regard to a porous material, the term “selectively permeable” refers to that which allows passage of certain molecules based upon size, surface- or other charge, hydrophilicity/phobicity, topology or other consideration.

As used herein, “retrievable” refers to capable of being recovered. According to the invention, a retrievable, substantially encapsulated, organized tissue can be recovered after implantation into a host mammal in an intact state such that the encapsulated tissue can be reimplanted or the organized tissue can be removed from the sleeve such that the organized tissue maintains its shape after being removed from the sleeve, and the organized tissue can be cultured in vitro under conditions which preserve its in vivo viability after being removed from the sleeve.

As used herein “maintains its shape” refers to an organized tissue which maintains its organized structure after being removed from the sleeve within which it is contained. As used herein with regard to an organized tissue in a sleeve, “maintains tension” refers to a force of at least 1 pdyne applied by the sleeve to the organized tissue, which force prevents changes in length of the organized tissue of greater than 5% of the starting length of the organized tissue, wherein such tension requires attachment of the first and second points of the organized tissue to first and second points of the sleeve material such that detachment at either point of the tissue from the sleeve results in shortening of the organized tissue or lengthening of the sleeve.

As used herein “retractile forces” refer to forces of at least 1 pdyne that cause an object to contract lengthwise (shorten).

As used herein “permselective” refers to a material having a pore size of approximately 5 to 50 nm. Such a material allows solute exchange at the level of proteins through the pores.

As used herein “microporous” refers to a material having a pore size of approximately 0.5 μm to 10. μm. Such a material allows protein exchange through the pores, but does not allow cell exchange through the pores.

As used herein “macroporous” refers to a material having a pore size of approximately 10 μm to 200 μm. Such a material allows cell passage through the pores as well as vascularization.

As used herein “mesh structure” refers to a material having a pore size of approximately 200μ to 10 mm Such a material allows direct contact between organized tissue and the host tissue, as well as vascularization. The mesh structure may, in certain preferred embodiments, encompass a large open weave structure.

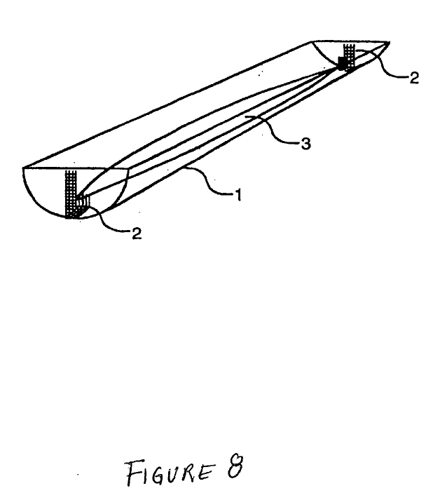

In accordance with a first preferred embodiment as shown in FIG. 9, an organized tissue 2 is positioned within a sleeve 4. Sleeve 4 is a bicompatible structure and, as illustrated in this embodiment, may have a substantially tubular or cylindrical shape. Organized tissue 2 is secured at a first longitudinal terminus, or point for attachment 6 to first end wall 8 of sleeve 4, and at a second longitudinal terminus, or point for attachment 10 to second end wall 12 of sleeve 4. As shown in FIG. 6, sleeve 4 is closed at both ends by end walls 8, 12. Sleeve 4 surrounds the organized tissue in at least one dimension and along a length of the organized tissue.

In certain embodiments, sleeve 4 is sufficiently flexible such that it will conform to the shape of organized tissue 2. Sleeve 4 may be comprised of a shrink wrap material or any suitable material having shape memory which will sufficiently conform to the shape of organized tissue 2.

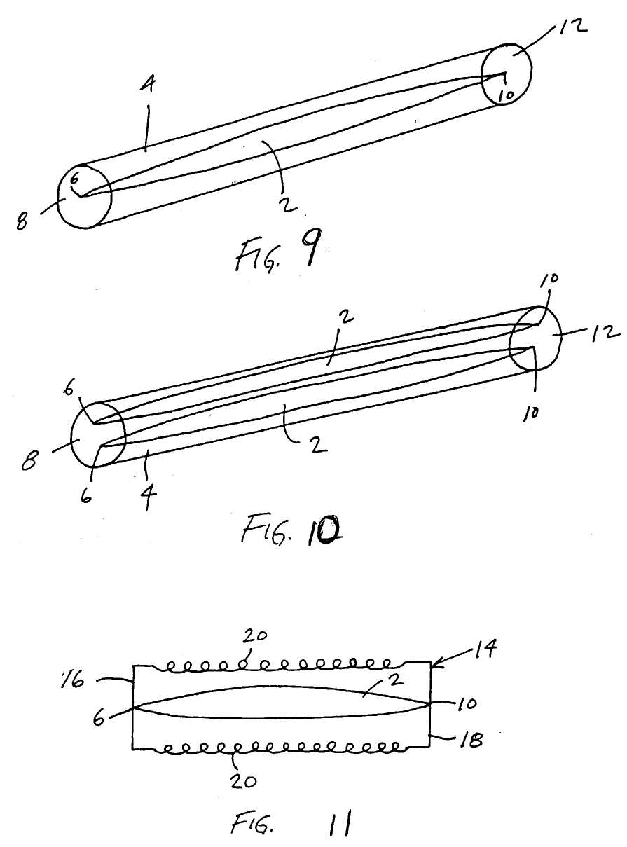

In certain preferred embodiments, as shown in FIG. 10, a second organized tissue 2 may be positioned within sleeve 4. As shown in FIG. 11, organized tissue 2 may be attached at first point for attachment 6 and second point for attachment 10 to a tension maintaining member 14.

In certain embodiments, sleeve 4 is preferably a preformed structure, having a predetermined shape and dimension prior to insertion of organized tissue therein or prior to the formation of organized tissue therein. Sleeve 4 is preferably formed of a porous material, wherein sleeve 4 is selectively permeable in order to allow access of small molecules and proteins while excluding larger molecules. Sleeve 4 may be permselective, having a pore size of approximately 5 to 50 nm and allowing solute exchange at the level of proteins through the pores. Sleeve 4 may be microporous, having a pore size of approximately 0.5 μm to 10 μm and allowing protein exchange through the pores, but not allowing cell exchange through the pores Sleeve 4 may be macroporous, having a pore size of approximately 10 μm to 200 μm and allowing cell passage through the pores as well as vascularization. Sleeve 4 may also have a mesh structure, with a pore size of approximately 200 μm to 10 mm and allowing direct contact between organized tissue and the host tissue, as well as vascularization.

The sleeve can be constructed from a material selected from the group including, but not limited to, polyacrylates, polymethyl-acrylates, polyalginate, polyvinyl alcohols, polyethylene oxide, polyvinylidene fluoride, polyvinylidenes, polyvinyl chloride, polyurethanes, polyurethane isocyanates, polystyrenes, polyamides, polyaspartate, polyglutamate, cellulose-based polymers, cellulose acetates, cellulose nitrates, polysulfones, polyphosphazenes, polyacrilonitriles, poly(acrilonitrile/covinyl chloride), stretched, woven, extruded or molded polytetrafluoroethylene, stretched, woven, extruded or molded polypropylene, stretched, woven, extruded or molded polyethylene, porous polyvinylidene fluoride, Angel Hair, silicon-oxygen-silicon matrices, polylsine and derivatives, copolymers and mixtures thereof The sleeve can also be constructed of natural materials including, but not limited to, collagen, extracellular matrix, intestinal mucosa, and metals including, but not limited to, stainless steel, tantalum, titanium and its alloys, and nitinol

Production of an Organized Tissue and Transfer to Sleeve

An organized tissueof the invention may be produced in vitro from the individual cells of a tissue of interest as described herein.

In the embodiment of the invention wherein the tissue having an in vivo-like gross and cellular morphology is grown in vitro, the vessel in which the tissue is grown also includes tissue attachment surfaces which are an integral part of or coupled to the vessel. Such a vessel may be constructed from a variety of materials which are compatible with the culturing of cells and tissues (e.g., capable of being sterilized and compatible with a particular solution of extracellular matrix components) and which are formable into three dimensional shapes approximating the in vivo gross morphology of a tissue of interest. The tissue attachment surfaces (e.g., stainless steel mesh, VELCRO™., or the like) are coupled to the vessel and positioned such that as the tissue forms in vitro the cells may adhere to and align between the attachment surfaces The tissue attachment surfaces may be constructed from a variety of materials which are compatible with the culturing of cells and tissues (e.g, capable of being sterilized, or having an appropriate surface charge, texture, or coating for cell adherence).

The tissue attachment surfaces may be coupled in a variety of ways to an interior or exterior surface of the vessel or sleeve. Alternatively, the tissue attachment surfaces may be coupled to the culture chamber such that they are positioned adjacent the vessel and accessible by the cells during tissue formation. In addition to serving as points of adherence, in certain tissue types (e g, muscle), the attachment surfaces allow for the development of tension by the tissue between opposing attachment surfaces. Moreover, where it is desirable to maintain this tension in vivo, the tissue adhered to the tissue attachment surfaces may be transferred to a sleeve according to the invention and the sleeved tissue is then implanted into an organism

Production of an Organized Tissue in a Sleeve

An organized tissue may be grown in a sleeve as follows. Organized tissue cells in a biocompatible physiological buffered solution are injected in a sleeve having a desired porosity. The sleeve is then placed in a petri dish containing a suitable media solution and maintained under controlled conditions for a number of days. The solution in which the petri dish is maintained may be periodically changed Thus, a kit according to the invention will include a sleeved organized tissue of the invention, comprising a sleeve containing one or more organized tissues, a biocompatible physiological buffered solution in which the organized tissue is maintained within the sleeve for from hours to days to 12 weeks without significant loss of bioactivity, and packaging materials therefor. The biocompatible physiological buffered solution includes, minimally, amino acids, vitamins, essential trace elements and also may include additional components such as growth factors, serum, and tissue extracts.

In a preferred embodiment, a force is applied by organized tissue 2 to sleeve 4, or vice versa, to maintain tension Thus, organized tissue 2 is longitudinally stretched and/or sleeve 4 is retracted when organized tissue 2 is attached at first and second points for attachment 6, 10, respectively, to sleeve 4. By introducing tension into organized tissue 2, the amount of bioactive compound produced by the tissue can be sustained long-term. Organized tissue 2 may also create retractile forces that reduce the length of sleeve 4.

In certain preferred embodiments, organized tissue 2 may be attached to a tension maintaining member rather than sleeve 4 itself. As shown in FIG. 11, organized tissue 2 may be attached at first point for attachment 6 and second point for attachment 10 to a tension maintaining member 14. In the illustrated embodiment, tension maintaining member 14 comprises first support member 16 and second support member 18 connected to one another by a pair of spring members 20. Organized tissue is anchored at first point for attachment 6 to first support member 16 and at second point for attachment 10 to second support member 18. Tension maintaining member 14 and organized tissue 2 anchored thereto can then be positioned within a sleeve 4. Thus, when organized tissue 2, which is anchored to tension maintaining member 14, is removed from sleeve 4, the organized tissue maintains its shape.

It is to be appreciated that sleeve 4 may, in certain preferred embodiments, be open at first end 8, at second end 12, or at both first end 8 and second end 12.

Sleeve 4, having organized tissue 2 contained therein, may be implanted into a mammal, e.g., a human Sleeve 4 and organized tissue 2 may then be retrieved at a later time from the site of implantation.

In accordance with another preferred embodiment, organized tissue can be produced in vitro by providing organized tissue and placing the organized tissue in a sleeve. The organized tissue may be provided by growing cells and placing the cells in a vessel in which the organized tissue is formed. The organized tissue may then be implanted in a mammal.

In accordance with another preferred embodiment, organized tissue can be produced in vitro by providing growing cells and placing the growing cells into a sleeve under conditions which permit the growing cells to form an organized tissue in the sleeve. The organized tissue is preferably substantially encapsulated within the sleeve. The organized tissue may then be implanted in a mammal.

In accordance with another preferred embodiment, protein may be provided to a mammal, e.g., a human. As a first step in this process, an organized tissue comprising cells which produce a protein is surrounded by a sleeve in at least one dimension and along a length of the organized tissue. The cells may be comprised of like species as the mammal (autologous or allogeneic), or different species (xenogeneic). The sleeved organized tissue is then implanted into a manmmal and the protein is produced in the mammal after the implanting. The sleeved organized tissue may then be removed from the mammal to terminate delivery of the protein. After removal, the organized tissue may be removed from the sleeve and cultured in vitro under conditions which preserve its in vitro viability The organized tissue may then be reinserted into a sleeve, and the sleeved organized tissue may be reimplanted into the mammal to deliver the protein to the mammal Alternatively, after removal, the sleeved organized tissue may be cultured in vitro under conditions which preserve its in vivo viability and reimplanted in the mammal.

The organized tissue may be provided by growing a plurality of mammalian cells in vitro, wherein at least a subset of the cells comprise a bioactive compound, the cells being mixed with an extracellular matrix to create a suspension The suspension may then be placed in a vessel to form an organized tissue of interest having a three dimensional cellular organization which is retained when implanted into a mammal. The tissue may then be inserted into a sleeve.

In accordance with another preferred embodiment, protein may be provided to a mammal, e.g., a human. As a first step in this process, a plurality of mammalian cells are grown in vitro. The cells may be comprised of like species as the mammal At least a subset of the cells comprise a DNA sequence operably linked to a promoter and encoding a protein, and wherein the cells are mixed with an extracellular matrix to create a suspension. The suspension is then placed in a vessel wherein the cells form an organized tissue of interest having a three dimensional cellular organization which is retained upon implantation into a mammal. The organized tissue is then inserted into a sleeve and then implanted into the mammal, whereby the protein is produced in the mammal and the protein is of a type or produced in an amount not normally produced by the cells in the organized tissue.

In accordance with another preferred embodiment, protein may be provided to a mammal, e.g., a human. As a first step in this process, a plurality of mammalian cells are grown in vitro. The cells may be comprised of like species or different species of the mammal. At least a subset of the cells comprise a DNA sequence operably linked to a promoter and encoding a protein, and the cells are mixed with an extracellular matrix to create a suspension. The suspension is then placed in a sleeve, wherein the cells form an organized tissue of interest having a three dimensional cellular organization which is retained upon implantation into a mammal. The sleeved organized tissue is then implanted into the mammal, and the protein is produced in the mammal. The protein is of a type or produced in an amount not normally produced by the cells in the organized tissue.

The sleeved organized tissue may, in certain preferred embodiments, be removed from the mammal to terminate delivery of the protein After removal of the sleeved organized tissue, the organized tissue may be removed from the sleeve and the organized tissue may be cultured in vitro under conditions which preserve its in vivo viability. After culturing, the organized tissue may be reinserted into a sleeve and the sleeved organized tissue may then be reimplanted into the mammal so that the protein is produced in the mammal. The sleeved tissue may be attached to a tether to enhance removal.

Alternatively, after removal of the sleeved organized tissue, the sleeved organized tissue may be cultured in vitro under conditions which preserve its in vivo viability and then reimplanted into the mammal so that the protein is produced in the mammal.

The sleeved organized tissue may be implanted into the tissue of origin of at least one of the cells comprising the organized tissue, or, alternatively, may be implanted into a tissue not of origin of cells comprising the organized tissue.

The protein may be expressed from a DNA sequence comprised of at least a subset of cells of the substantially encapsulated organized tissue. A second protein may be expressed from a second DNA sequence

The sleeved organized tissue may comprise skeletal muscle cells, fibroblast cells, or a combination of skeletal muscle cells and fibroblast cells or other cells The sleeved organized tissue may comprise muscle fibers.

Use of Sleeved Organized Tissue to Deliver Bioactive Compound to an Organism

A bioactive compound may be delivered to an organism using a device such as a catheter into which the sleeved organized tissue that produces the bioactive compound has been placed, and after catheterization, implanting the sleeved organized tissue into the organism. Alternatively, the sleeved organized tissue may be directly implanted into the organism using, e.g., surgical forceps, pipette, cannula, trocar, fibrin or other glues, manually or pulling via a suture.

A variety of bioactive compounds may be delivered by this method, and they may function through intracellular (i.e., within the cells of the organized tissue or organoid), endocrine, autocrine, or paracrine mechanisms. Moreover, the organized tissue may deliver multiple bioactive compounds either simultaneously or sequentially (e.g., one bioactive compound mediates the delivery of another). Liberation of the bioactive compound from the cells of the organized tissue may occur by either passive or active processes (e.g., diffusion or secretion).

For example, the bioactive compound may be a hormone, growth factor, or the like which is produced and liberated by the cells of the organized tissue to act locally or systemically on host tissues. Alternatively, the bioactive compound may function within the cells or on the surface of the cells of the organized tissue to enhance the uptake or metabolism of compounds from the host tissue or circulation (e.g., lactic acid, low density lipoprotein) Where the organized tissue serves as a functional and structural adjunct to the host tissue, delivery of growth factors by autocrine or paracrine mechanisms may enhance the integration of the organized tissue into host tissues. Similarly, where multiple bioactive compounds are produced by the organized tissue, autocrine delivery of one of the bioactive compounds may be used to regulate the production of one or more of the other bloactive compounds.

The organized tissue may be implanted at a desired anatomical location within the organism. For example, the organized tissue may be implanted in the same or a different tissue from the tissue of origin of at least one of the individual cells The location of implantation depends, in part, upon the identity of the particular bioactive compound to be delivered. For example, an organized tissue acting as an endocrine organ may be implanted in or adjacent a highly vascularized host tissue Alternatively, an organized tissue acting as a paracrine organ is preferably implanted in or adjacent to the host tissue to which the bioactive compound is to be delivered.

The sleeved organized tissue may be implanted by attachment to a host tissue or as a free floating sleeved organized tissue. In addition, attached organized tissues may be implanted with or without the tissue attachment surfaces used for in vitro tissue formation. Tissues responsive to mechanical forces are preferably implanted by attaching directly to the host tissue or by implanting the organized tissue coupled to the attachment surfaces so that the organized tissue is exposed to mechanical forces in vivo. For example, skeletal muscle organized tissue is preferably implanted by attachment to the host tissue under tension along a longitudinal axis of the organized tissue. Moreover, the organized tissue may be permanently or temporarily implanted. Permanent implantation may be preferred, for example, where the organized tissue produces a bioactive compound which corrects a systemic metabolic error (e.g, delivery of insulin to treat diabetes), whereas temporary implantation may be preferred where only transient delivery of a bioactive compound is desired (e.g., delivery of a growth factor to enhance wound healing) Furthermore, because organized tissue may be implanted, removed, and maintained in vitro, bioactive compounds may be delivered intermittently to the same or a different location in the organism For example, a skeletal muscle organized tissue produced from the cells of a human patient (e.g, an autograft or allograft) may be implanted at a first anatomical location for a defined period and subsequently implanted at a second location at or after the time of removal.

Vasculogenic Factors Useful According to The Invention

A vasculogenic factor useful according to the invention includes but is not limited to any of the following: VEGF A, B, C. D, E (Vascular endothelial growth factor), FGF 1, 2, 3, 4, 5 (Fibroblast growth factor), PDGF AA, BB, AB (platelet derived growth factor), angiopoeitins, MCP (macrophage chemoattractant protein), EPO (erythropoeitin), IL 1-22 (interleukins), ephrins, and any of the vasculogenic factors included in Table I.

Promoters

Promoters useful according to the invention include constitutive viral promoters such as (1) long terminal repeat promoter (Bonham et al Human Gene Therapy 7, 1423, 1996); and (2) cytomegalovirus promoter (Yogalingam et al BBA 1453, 284, 1999), Muscle specific promoters such as (1) skeletal alpha actin promoter (Muscat et al Gene Expres. 2, 241, 1992); and (2) myoglobin promoter (Devlin et al., J. Biol. Chem., 264: 138967) and inducible promoters such as tetracycline-inducible promoter (Sturtz et al Gene 221, 279, 1998).

Vectors

As used herein, the term “expression vector” or “vector” refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome-binding site, often along with other sequences. Useful expression vectors include, but are not limited to, retroviral vectors, for example, pLgXSN (gift of Dusty Miller, Fred Hutchinson Cancer Center, Seattle, Wash.) and MFGP_{L} expression system, His Fusion system, pBAD vectors from Invitrogen (Carlsbad, Calif.); pTrc vectors from Amersham Biosciences (Piscataway, N.J.); pALTER vectors from Promega (Madison, Wis.); pBH, pBV, pBX vectors from Roche Molecular Biochemicals (Summerville, N.J.); pCAL vectors and pET vectors from Stratagene (La Jolla, Calif.); and pET vectors from Novagen (Madison, Wis.).

Bioactive Compounds

The invention provides for delivery of any bioactive compound as defined herein. The invention provides for delivery of a bioactive compound directly into the circulation and is therefore useful for the delivery of large, unstable molecules, for example Factor VIII

“Bioactive compounds” according to the invention include proteins, fusion proteins, antibodies, antibody fragments, viral and non-viral vectors, RNA and DNA.

Bioactive compounds of interest for use with the present invention include receptors, enzymes, ligands, regulatory factors, and structural proteins. Therapeutic proteins including nuclear proteins, cytoplasmic proteins, mitochondrial proteins, secreted proteins, plasmalemma-associated proteins, serum proteins, viral antigens and proteins, bacterial antigens, protozoal antigens and parasitic antigens are also useful according to the invention.

Therapeutic proteins useful according to the invention also include lipoproteins, glycoproteins, phosphoproteins. Proteins or polypeptides which can be expressed using the methods of the present invention include hormones, growth factors, neurotransmitters, enzymes, clotting factors, apolipoproteins, receptors, drugs, oncogenes, tumor antigens, tumor suppressors, structural proteins, viral antigens, parasitic antigens and bacterial antigens. Specific examples of these compounds include proinsulin (GenBank #E00011), growth hormone, dystrophin (GenBank # NM_{—}007124), androgen receptors, insulin-like growth factor I (GenBank #NM_{—}00875), insulin-like growth factor II (GenBank #X07868) insulin-like growth factor binding proteins, epidermal growth factor TGF-α(GenBank #E02925), TGF-β (GenBank #AW008981), PDGF (GenBank #NM_{—}002607), angiogenesis factors (acidic fibroblast growth factor (GenBank #E03043), basic fibroblast growth factor (GenBank #NM _{—}002006) and angiogenin (GenBank #M11567), matrix proteins (Type IV collagen (GenBank #NM_{—}000495), Type VII collagen (GenBank #NM_{—}000094), laminin (GenBank # J03202), phenylalanine hydroxylase (GenBank #K03020), tyrosine hydroxylase (GenBank #X05290), oncogenes (ras (GenBank #AF 22080), fos (GenBank #k00650), myc (GenBank #J00120), erb (GenBank #X03363), src (GenBank #AH002989), sis GenBank #M84453), jun (GenBank #J04111)), E6 or E7 transforming sequence, p53 protein (GenBank #AH007667), Rb gene product (GenBank #m19701), cytokine receptor, I1-1 (GenBank #m54933), IL-6 (GenBank #e04823), IL-8 (GenBank #119591), viral capsid protein, and proteins from viral, bacterial and parasitic organisms which can be used to induce an immunologic response, and other proteins of useful significance in the body.

The compounds which can be incorporated are only limited by the availability of the nucleic acid sequence for the protein or polypeptide to be incorporated. One skilled in the art will readily recognize that as more proteins and polypeptides become identified they can be integrated into the DNA constructs of the invention and used to transform or infect cells useful for producing an organized tissue according to the methods of the present invention.

The invention also provides for bioactive compounds that are vaccines, anti-infectives and anti-inflammatories (for example TNF-α). Additional bioactive compounds are included in

TABLE II

| | | | Approval |
|---|---|---|---|
| Product | Company | Application (use) | Date |
| Abbott HTLV-I/ | Abbott Laboratories | EIA for detection of HTLV- | August 1997 |
| HTLV-II EIA | | I/HTLV-II | |
| | | antibodies in serum or plasma | |
| Abelcett ® (amphotericin B | The Liposome | Treatment of invasive fungal | November 1995 |
| lipid complex injection) | Company, Inc. | infections in patients who are | |
| | | refractory to or intolerant of | |
| | | conventional amphotericin B | |
| | | (lipid-complex drug delivery | |
| | | system) | |
| Abreva ™ (docosanol) | AVANIR | Topical treatment of recurrent | July 2000 |
| | Pharmaceuticals | cold sores (herpes simplex | |
| | and | infection) | |
| | GlaxoSmithKline, | | |
| | Inc. | | |
| Argatroban | Texas Biotechnology | Anticoagulant for prophylaxis | June 2000 |
| | Corporation and | or treatment of thrombosis in | April 2002 |
| | GlaxoSmithKline, | patients with heparin-induced | |
| | Inc. | thrombocytopenia; Heparin- | |
| | | induced thrombocytopenia in | |
| | | patients undergoing | |
| | | percutaneous coronary | |
| | | interventions | |
| Actimmune ® | Genentech, Inc., and | Treatment of chronic | December 1990 |
| (interferon gamma-1b) | InterMune | granulomatous disease; | February 2000 |
| | Pharmaceuticals, | treatment of severe, malignant | |
| | Inc. | osteopetrosis | |
| Activase ®/Cathflo ™ | Genentech, Inc. | Treatment of acute myocardial | November 1987 |
| Activase ® | | infarction; acute massive | June 1990 |
| (alteplase; recombinant | | pulmonary embolism; acute | June 1996 |
| tissue plasminogen | | ischemic stroke within first | September 2001 |
| activator) | | three hours of symptom onset; | |
| | | Dissolution of clots in central | |
| | | venous access devices | |
| | | (Cathflo ™ Activase ®) | |
| Adagen ® | Enzon, Inc. | Treatment of severe combined | March 1990 |
| (adenosine deaminase) | | immunodeficiency disease | |
| | | (SCID) | |
| Albutein ® | Alpha Therapeutic | Treatment of hypovolmeic | January 1986 |
| (human albumin) | Corporation | shock; an adjunct in | |
| | | hemodialysis; used in | |
| | | cardiopulmonary bypass | |
| | | procedures | |
| Alferon N ® | Interferon Sciences, | Treatment of genital warts | October 1989 |
| (interferon alfa-N3, human | Inc | | |
| leukocyte derived) | | | |
| Alphanate ® | Alpha Therapeutic | Treatment of hemophilia A or | February 1997 |
| (human antihemophilic | Corporation | acquired factor VII deficiency | |
| factor) | | | |
| AlphaNine ® SD | Alpha Therapeutic | Prevention and control of | July 1996 |
| (virus-filtered human | Corporation | bleeding in patients with factor | |
| coagulation factor IX) | | IX deficiency due to | |
| | | hemophilia B | |
| AmBisome ® | Gilead Sciences, | Treatment of fungal infections | August 1997 |
| (liposomal amphotericin B) | Inc. | in patients with depressed | June 2000 |
| | (from NeXstar | immune function and with | |
| | Pharmaceuticals) | fever of unknown origin; | |
| | and Fujisawa | treatment of cryptococcal | |
| | Healthcare | meningitis in HIV-infected | |
| | | patients | |
| AMPHOTEC ® | SEQUUS | Second-line treatment of | November 1996 |
| (lipid-based colloidal | Pharmaceuticals | invasive aspergillosis | |
| dispersion of amphotericin | | infections | |
| B) | | | |
| AndroGel ™ | Unimed | Testosterone-replacement | February 2000 |
| (testosterone) | Pharmaceuticals, | therapy in males with | |
| | Inc. (subsidiary of | testosterone deficiency | |
| | Solvay | | |
| | Pharmaceuticals) | | |
| Angiomax ® | The Medicines | Anticoagulant in conjunction | December 2000 |
| (bivalirudin) | Company | with aspirin in patients with | |
| | | unstable angina undergoing | |
| | | percutaneous transluminal | |
| | | coronary angioplasty | |
| Apligraf ® | Organogenesis, Inc. | Treatment of venous leg | May 1998 |
| (living, from collagen, | | ulcers; treatment of human | June 2000 |
| fibroblasts and | | skin substitute diabetic foot | |
| keratinocytes) | | ulcers | |
| Aranesp ™ | Amgen | Anemia associated with | September 2001 |
| (darbepoetin | | chronic renal failure; | July 2002 |
| alfa; recombinant | | Chemotherapy-induced | |
| erythropoiesis-stimulating | | anemia in patients with non- | |
| protein) | | myeloid malignancies | |
| AVINZA ™ | Ligand | Once-daily treatment of | March 2002 |
| (morphine sulfate | Pharmaceuticals | moderate to severe pain in | |
| extended-release | and Elan Corp. plc | patients who require | |
| capsules) | | continuous opioid therapy for | |
| | | an extended period of time | |
| Avonex ® | Biogen | Treatment of relapsing- | May 1996 |
| (recombinant interferon | | remitting forms of multiple | |
| beta 1-alpha) | | sclerosis | |
| BeneFix ™ | Genetics Institute | Treatment of hemophilia B | February 1997 |
| Coagulation factor IX | | | |
| (recombinant) | | | |
| Betaseron ® | Berlex Laboratories | Treatment of relapsing- | August 1993 |
| (recombinant interferon | and | remitting multiple sclerosis | |
| beta 1-B) | Chiron Corporation | | |
| Bioclate ™ | Centeon | Treatment of hemophilia A for | December 1993 |
| (recombinant | | the prevention and control of | |
| antihemophilic factor) | | hemorrhagic episodes; | |
| | | perioperative management of | |
| | | patients with hemophilia A | |
| BioTropin ™ | Biotech General | Treatment of human growth | May 1995 |
| | | hormone deficiency in children | |
| Campath ® | Ilex Oncology Inc., | B-cell chronic lymphocytic | May 2001 |
| (alemtuzumab; | Millennium | leukemia in patients who have | |
| recombinant monoclonal | Pharmceuticals Inc. | been treated with alkylating | |
| antibody against CD52 | and Berlex | agents and who have failed | |
| glycoprotein) | Laboratories Inc. | fludarabine therapy | |
| Carticel ™ | Genzyme | Reconstruction of knee | August 1997 |
| (autologous cultured | | cartilage damage | |
| chondrocytes) | | | |
| Ceredase ®/Cerezyme ® | Genzyme | Treatment of type 1 Gaucher's | April 1991 |
| (alglucerase/recombinant | | disease | May 1994 |
| alglucerase) | | | |
| Chiron RIBA ® | Chiron Corporation | Detection of antibodies to | February 1999 |
| HCV 3.0 Strip Immunoblot | and | hepatitis C in human serum or | |
| Assay | Johnson & Johnson | plasma | |
| CroFab ™ (crotalidae | Protherics, plc, and | Rattlesnake anti-venom | October 2000 |
| polyvalent immune Fab, | Altana | | |
| ovine) | | | |
| CytoGam ® | MedImmune, Inc. | Prevention of cytomegalovirus | December 1998 |
| (CMV immune globulin IV) | | (CMV) in kidney transplant | April 1990 |
| | | patients; prevention of CMV | |
| | | disease associated with | |
| | | kidney, lung, liver, pancreas | |
| | | and heart transplants | |
| DaunoXome ® | NeXstar | First-line treatment for HIV- | April 1996 |
| (liposomal form of the | Pharmaceuticals | related Kaposi's sarcoma | |
| chemotherapeutic agent | | | |
| daunorubicin) | | | |
| Depocyt ™ | Depotech | Treatment of lymphomatous | April 1999 |
| | (SkyePharma) | meningitis | |
| | and Chiron | | |
| | Corporation | | |
| Dermagraft ® | Advanced Tissue | Diabetic foot ulcers | September 2001 |
| (human-based, tissue- | Sciences Inc. and | | |
| engineered living dermal | Smith & Nephew plc | | |
| substitute) | | | |
| DigiFab ™ | Protherics plc | Digoxin toxicity | September 2001 |
| (digoxin immune fab | | | |
| [ovine]) | | | |
| Doxil ® | Alza | Second-line therapy for | November 1995 |
| (liposomal formulation of | | Kaposi's sarcoma in AIDS | June 1999 |
| doxorubicin hydrochloride) | | patients; metastatic carcinoma | |
| | | of the ovary in patients with | |
| | | disease that is refractory to | |
| | | both paclitaxel- and platinum- | |
| | | based chemotherapy regimens | |
| Eligard ™ | Atrix Laboratories | Advanced prostate cancer | January 2002 |
| (slow-release formulation | and Sanofi- | | (additional |
| of leuprolide acetate) | Synthelabo | | formulation |
| | | | cleared in |
| | | | July 2002) |
| Elitek ® | Sanofi-Synthelabo | Management of plasma uric | July 2002 |
| (rasburicase) | | acid levels in pediatric | |
| | | chemotherapy patients | |
| Enbrel ® | Amgen and Wyeth | Treatment of moderate to | November 1998 |
| (etanercept) | | severely active rheumatoid | May 1999 |
| | | arthritis in patients who have | June 2000 |
| | | had an inadequate response | January 2002 |
| | | to one or more disease- | |
| | | modifying antirheumatic drugs; | |
| | | treatment of polyarticular | |
| | | course juvenile rheumatoid | |
| | | arthritis; treatment as a first- | |
| | | line therapy for moderate to | |
| | | severe active rheumatoid | |
| | | arthritis; Reduction of signs | |
| | | and symptoms of active | |
| | | arthritis in patients with | |
| | | psoriatic arthritis | |
| Engerix-B ® | GlaxoSmithKline | Hepatitis B vaccine; adults | September 1989 |
| (recombinant hepatitis B | | with chronic hepatitis C | August 1998 |
| vaccine) | | infection | |
| Epogen ® | Amgen | Treatment of anemia | June 1989 |
| (epoietin alfa) | | associated with chronic renal | July 1999 |
| | | failure and anemia in Retrovir- | |
| | | treated HIV-infected patients; | |
| | | pediatric use | |
| Fertinex ™ | Serono Laboratories | Treatment of female infertility | August 1996 |
| | | to stimulate ovulation in | |
| | | women with ovulatory | |
| | | disorders and in women | |
| | | undergoing assisted | |
| | | reproductive technologies | |
| Focalin ™ | Celgene Corp. and | Attention deficit hyperactivity | November 2001 |
| (dexmethylphenidate | Novartis | disorder | |
| hydrochloride; refined | Pharmaceuticals | | |
| version of methylphenidate | Corp. | | |
| containing only the active | | | |
| isomer) | | | |
| Follistim ™ | Organon (unit of | Recombinant follicle- | September 1997 |
| (follitropin beta for | Akzo Nobel) | stimulating hormone for | February 2002 |
| injection) | | treatment of infertility; | |
| | | Induction of spermatogenesis | |
| | | in men with primary and | |
| | | secondary hypo-gonadotropic | |
| | | hypogonadism in whom the | |
| | | cause of infertility is not due to | |
| | | primary testicular failure | |
| FortaFlex ™ | Organogenesis Inc. | Rotator cuff repair | April 2002 |
| (bioengineered collagen | and Biomet Inc. | | |
| matrix) | | | |
| Frova ™ | Vernalis Group plc | Migraine | November 2001 |
| (frovatriptan succinate) | and Elan Corp. plc | | |
| GenoTropin ® | Pharmacia & Upjohn | Treatment of growth hormone | August 1995 |
| (recombinant form of | | deficiency in children; growth | November 1997 |
| human somatropin) | | hormone deficiency in adults; | July 2001 |
| | | Long-term treatment of growth | |
| | | failure in children born small | |
| | | for gestational age who fail to | |
| | | catch up by age 2 | |
| Geref ® | Serono Laboratories | Treatment of growth hormone | October 1997 |
| | | deficiency in children with | |
| | | growth failure | |
| Gleevec ™ | Novartis | Chronic myeloid leukemia in | May 2001 |
| (imatinib mesylate) | Pharmaceuticals | blast crisis, accelerated phase, | February 2002 |
| | Corp | or in chronic phase after failure | |
| | | of interferon-alpha therapy; | |
| | | Patients with Kit (CD117) | |
| | | positive unresectable and/or | |
| | | metastatic malignant | |
| | | gastrointestinal stromal tumors | |
| Gonal-F ® | Serono Laboratories | Treatment of infertility in | September 1998 |
| (follitropin alfa) | | women not due to primary | June 2000 |
| | | ovarian failure; treatment of | |
| | | infertility in men and women | |
| Helixate ® | Aventis | Factor VIII for treatment of | February 1994 |
| (recombinant | | hemophilia A; second- | June 2000 |
| antihemophilic factor) | | generation factor VIII | |
| | | formulated with sucrose for | |
| | | treatment of hemophilia A | |
| Herceptin ® | Genentech, Inc. | Treatment of patients with | September 1998 |
| (trastuzumab) | | metastatic breast cancer | |
| | | whose tumors overexpress the | |
| | | HER2 protein | |
| Hextend ® | BioTime, Inc. | Plasma volume expander for | March 1999 |
| (hetastarch) | | treatment of hypovolemia | |
| | | during surgery | |
| Humalog ® (recombinant | Eli Lilly & Company | Treatment of diabetes | June 1996 |
| insulin) | | | |
| Humate-P ® | Centeon | Treatment and prevention of | April 1999 |
| (antihemophiliofactor/von | | bleeding episodes in | |
| Willebrand factor complex- | | hemophilia A adult patients; | |
| human) | | spontaneous and trauma- | |
| | | induced bleeding episodes in | |
| | | severe von Willebrand disease | |
| | | in adult and pediatric patients, | |
| | | and in mild and moderate von | |
| | | Willebrand disease where use | |
| | | of desmopressin is known or | |
| | | suspected to be inadequate | |
| Humatrope ® | Eli Lilly & Company | Treatment of growth hormone | August 1996 |
| (recombinant | | deficiency in children; | March 1997 |
| somatotropin) | | somatotropin deficiency | |
| | | syndrome in adults | |
| Humulin ® | Eli Lilly & Company | Treatment of diabetes | October 1982 |
| (recombinant human | | | |
| insulin) | | | |
| Imagent ® | Alliance | Contrast agent for anatomical | June 2002 |
| (perflexane lipid | Pharmaceutical | imaging of the heart | |
| microspheres) | Corp., Cardinal | | |
| | Health Inc. and | | |
| | InChord | | |
| | Communications | | |
| | Inc. | | |
| Infergen ® | Amgen | Treatment of hepatitis C (HCV) | October 1997 |
| (interferon alfacon-1) | | in patients 18 years or older | December 1999 |
| | | with compensated liver | |
| | | disease who have anti-HCV | |
| | | serum antibodies and/or the | |
| | | presence of HCV RNA; | |
| | | subsequent treatment of HCV- | |
| | | infected patients who have | |
| | | tolerated an initial course of | |
| | | interferon therapy | |
| INTEGRA ® | Integra Life | Dermal scar contractures | May 2002 |
| Dermal Regeneration | Sciences Holding | | |
| Template | Corp and Ethicon | | |
| | Inc. (a unit of | | |
| | Johnson & Johnson) | | |
| Integrilin ™ | COR Therapeutics, | Treatment of patients with | May 1998 |
| (eptifibatide for injection) | Inc., and Schering- | acute coronary syndrome and | September 1999 |
| | Plough Corporation | angioplasty; including patients | June 2001 |
| | | who, are to be managed | |
| | | medically and those | |
| | | undergoing percutaneous | |
| | | coronary intervention; Acute | |
| | | coronary syndrome, including | |
| | | both patients managed | |
| | | medically and those | |
| | | undergoing percutaneous | |
| | | coronary intervention | |
| Intron A ® | Schering-Plough | Treatment of hairy cell | June 1986 |
| (alpha-interferon) | Corporation | leukemia; genital warts; AIDS- | June 1988 |
| | | related Kaposi's sarcoma; | November 1988 |
| | | non-A, non-B hepatitis; | February 1991 |
| | | hepatitis B; chronic malignant | July 1992 |
| | | melanoma; extended therapy | December 1995 |
| | | for chronic viral hepatitis C; | March 1997 |
| | | treatment for follicular | November 1997 |
| | | lymphoma in conjunction with | August 1998 |
| | | chemotherapy; treatment of | |
| | | hepatitis B in pediatric patients | |
| Kineret ™ | Amgen | Moderately to severely active | November 2001 |
| (anakinra; recombinant | | rheumatoid arthritis in adult | |
| form of non-glycosylated | | patients who have failed | |
| human interleukin-1 | | disease-modifying anti- | |
| receptor antagonist) | | rheumatic drugs | |
| Kogenate ® FS | Bayer Corporation | Factor VII for treatment of | September 1989 |
| (recombinant | | hemophilia A; second- | June 2000 |
| antihemophilic factor) | | generation factor VIII | |
| | | formulated with sucrose for | |
| | | treatment of hemophilia A | |
| Lantus ® | Aventis | Biosynthetic basal insulin for | April 2000 |
| (insulin glargine) | | adult and pediatric patients | |
| | | with type 2 diabetes | |
| Leukine ® | Immunex | Treatment of autologous bone | March 1991 |
| (yeast-derived | Corporation | marrow transplantation; | September 1995 |
| GMCSF)/Leukine Liquid | | treatment of white blood cell | November 1995 |
| | | toxicities following induction | December 1995 |
| | | chemotherapy in older patients | November 1996 |
| | | with acute myelogenous | |
| | | leukemia; for use following | |
| | | allogenic bone marrow | |
| | | transplantation from HLA- | |
| | | matched related donors; for | |
| | | use mobilizing peripheral | |
| | | blood progenitor cells and for | |
| | | use after PBPC | |
| | | transplantation; (Leukine | |
| | | Liquid) ready-to-use | |
| | | formulation in a multidose vial | |
| Luestatin ™ | Ortho Biotech, Inc. | First-line treatment of hairy cell | March 1993 |
| (cladribine or 2-CDA) | | leukemia | |
| Luxiq ™ | Connetics | Relief of inflammatory and | February 1999 |
| (betamethasone) | Corporation | pruritic manifestations of | |
| | | corticosteroid-responsive | |
| | | dermatoses of the scalp | |
| LYMErix ™ | SmithKline Beecham | Prevention of Lyme disease | December 1998 |
| (recombinant OspA) | Biologicals | | |
| Metadate ® CD | Celltech | Attention deficit hyperactivity | April 2001 |
| (bi-phasic release | Pharmaceuticals Inc. | disorder | |
| formulation of | | | |
| methylphenidate) | | | |
| Mylotarg ™ | Celltech | Human antibody linked to | May 2000 |
| (gemtuzumab ozogamicin) | Chiroscience and | calicheamicin | |
| | Wyeth-Ayerst | (chemotherapeutic) for | |
| | (American Home | treatment of CD33 positive | |
| | Products | acute myeloid leukemia in | |
| | Corporation) | patients 60 and older in first | |
| | | relapse who are not | |
| | | considered candidates for | |
| | | cytotoxic chemotherapy | |
| Myobloc ™ | Elan Corporation | Treatment of cervical dystonia | December 2000 |
| (botulinum toxin type B) | | | |
| Nabi-HB ™ | Nabi | Treatment of acute exposure | March 1999 |
| (hepatitis B immune | | to HbsAg, perinatal exposure | |
| globulin-human) | | of infants born to HbsAg- | |
| | | positive mothers, sexual | |
| | | exposure to HbsAg-positive | |
| | | persons and household | |
| | | exposure of infants to persons | |
| | | with acute hepatitis B | |
| Natrecor ® | Scios Inc. | Acutely decompensated | August 2001 |
| (nesiritide; recombinant | | congestive heart failure with | |
| form of human B-type | | shortness of breath at rest or | |
| natriuretic peptide) | | with minimal activity | |
| Neulasta ™ | Amgen | Prevention of infection as | January 2002 |
| (pegfilgrastim) | | manifested by febrile | |
| | | neutropenia in cancer patients | |
| | | receiving chemotherapy | |
| Neumega ® | Genetics Institute | Prevention of severe | November 1997 |
| (oprelvekin) | | chemotherapy-induced | |
| | | thrombocytopenia in cancer | |
| | | patients | |
| Neupogen ® | Amgen | Treatment of chemotherapy- | February 1991 |
| (filgrastim) | | induced neutropenia; bone | June 1994 |
| | | marrow transplant | December 1994 |
| | | accompanied neutropenia; | December 1995 |
| | | severe chronic neutropenia; | April 1998 |
| | | autologous bone marrow | |
| | | transplant engraftment or | |
| | | failure; mobilization of | |
| | | autologous PBPCs after | |
| | | chemotherapy | |
| Norditropin ® | Novo Nordisk | Treatment of growth hormone | May 1995 |
| | | deficiency in children | |
| Novantrone ® | Immunex | Treatment of acute | December 1987 |
| (mitoxantrone | Corporation | nonlymphocytic leukemia; | November 1996 |
| hydrochloride) | | hormone refractory prostate | February 2000 |
| | | cancer; secondary progressive | |
| | | multiple sclerosis | |
| Novolin ® | Novo Nordisk | Treatment of diabetes | October 1982 |
| (recombinant human | | | |
| insulin) | | | |
| NovoLog ® | Novo Nordisk | Insulin analog for adults with | May 2000 |
| (insulin aspart) | | diabetes mellitus; For pump | December 2001 |
| | | therapy in diabetes | |
| NovoSeven ® | Novo Nordisk | Treatment of bleeding | March 1999 |
| (coagulation factor VIIa) | | episodes in hemophilia A or B | |
| | | patients with inhibitors to factor | |
| | | VIII or factor IX | |
| Nutropin Depot ™ | Genentech, Inc., and | Long-acting dosage form of | December 1999 |
| (somatropin for injectable | Alkermes, Inc. | recombinant growth hormone | |
| suspension) | | (one or two doses a month) for | |
| | | pediatric growth hormone | |
| | | deficiency | |
| Nutropin ®/ | Genentech, Inc. | Treatment of growth hormone | November 1993 |
| Nutropin AQ ® (somatropin | | deficiency in children; growth | January 1994 |
| rDNA) | | hormone deficiency in adults; | January 1996 |
| | | growth failure associated with | December 1996 |
| | | chronic renal insufficiency prior | December 1999 |
| | | to kidney transplantation; short | |
| | | stature associated with Turner | |
| | | Syndrome; to improve spine | |
| | | bone mineral density observed | |
| | | in childhood-onset adult | |
| | | growth hormone-deficient | |
| | | patients and to increase serum | |
| | | alkaline phosphatase | |
| Olux ™ (clobetasol | Connetics | Short-term topical treatment of | May 2000 |
| proprionate .05% foam) | Corporation | moderate to severe | |
| | | dermatoses of the scalp | |
| Oncaspar ® | Enzon, Inc., and | Treatment of acute | February 1994 |
| (PEG-L-asparaginase) | Rhone- Poulenc | lymphoblastic leukemia in | |
| | Rorer | patients who are | |
| | | hypersensitive to native forms | |
| | | of L-asparaginase | |
| Ontak ® | Ligand | Treatment of patients with | February 1999 |
| (denileukin diftitox) | Pharmaceuticals, | persistent or recurrent | |
| | Inc. | cutaneous T-cell lymphoma | |
| | | whose malignant cells express | |
| | | the CD25 component of the | |
| | | interleukin-2 receptor | |
| OrCel ™ | Ortec International | For patients with recessive | February 2001 |
| (composite cultured skin; | Inc. | dystrophic epidemolysis | August 2001 |
| bi-layered cellular matrix) | | bullosa undergoing hand | |
| | | reconstruction surgery; | |
| | | Treatment of donor site | |
| | | wounds in burn victims | |
| Orfadin ® | Swedish Orphan | Hereditary tyrosinemia Type 1 | January 2002 |
| (nitisinone) | International AB and | | |
| | Rare Disease | | |
| | Therapeutics Inc. | | |
| Orthoclone OKT3 ® | Ortho Biotech, Inc. | Reversal of acute kidney | June 1986 |
| (muromonab-CD3) | | transplant rejection | |
| Ovidrel ® | Serono Laboratories | Treatment of infertility in | September 2000 |
| (recombinant human | | women | |
| chorionic gonadotropin) | | | |
| Pacis ® | BioChem Pharma | Bladder cancer | March 2000 |
| (live attenuated Bacillus | and UroCor, Inc. | immunotherapy | |
| Calmette-Guerin) | | | |
| Panretin ® | Ligand | The topical treatment of | February 1999 |
| (alitretinoin) | Pharmaceuticals, | cutaneous lesions of patients | |
| | Inc. | with AIDS-related Kaposi's | |
| | | sarcoma | |
| PEG-Intron ™ | Enzon Inc. and | Monotherapy for chronic | January 2001 |
| (pegylated version of | Schering-Plough | hepatitis C; Combination | August 2001 |
| recombinant interfreron | Corp. | therapy with Rebetol for | |
| alfa-2b) | | treatment of hepatitis C in | |
| | | patients with compensated | |
| | | liver disease | |
| Photofrin ® (Porfimer | Ligand | Palliative treatment of totally | November 1995 |
| sodium) | Pharmaceuticals, | and partially obstructing | |
| | Inc. (licensed from | cancers of esophagus | |
| | QLT | | |
| | Phototherapeutics) | | |
| Prandin ™ (repaglinide) | Novo Nordisk | Anti-diabetic agent for | December 1997 |
| | | treatment of type 2 diabetes | |
| Prevnar ® (diphtheria CRM | American Home | Vaccine for infants and | February 2000 |
| 197 protein) | Products | toddlers, 12-15 months, to | |
| | Corporation- Wyeth- | prevent invasive | |
| | Lederle Vaccines | pneumococcal disease | |
| Procrit ® (epoietin alfa) | Ortho Biotech, Inc. | Treatment of anemia in ALT- | December 1990 |
| | | treated HIV-infected patients; | April 1993 |
| | | anemia in cancer patients on | December 1996 |
| | | chemotherapy; for use in | |
| | | anemic patients scheduled to | |
| | | undergo elective noncardiac, | |
| | | nonvascular surgery | |
| Proleukin, IL-2 ® | Chiron Corporation | Treatment of kidney | May 1992 |
| (aldesleukin) | | carcinoma/treatment of | January 1998 |
| | | metastatic melanoma | |
| Protropin ® (somatrem) | Genentech, Inc. | Treatment of growth hormone | October 1985 |
| | | deficiency in children | |
| PROVIGIL ® (modafinil) | Cephalon, Inc. | To improve wakefulness in | December 1998 |
| Tablets | | patients with excessive | |
| | | daytime sleepiness (EDS) | |
| | | associated with narcolepsy | |
| Pulmozyme ® (dornase, | Genentech, Inc. | Treatment of mild to moderate | December 1993 |
| alfa recombinant) | | cystic fibrosis; advanced cystic | December 1996 |
| | | fibrosis; pediatric use in infants | March 1998 |
| | | 3 months to 2 years and | |
| | | children 2 to 4 years old | |
| Rebetron ™ (combination | Schering-Plough | Combination therapy for | June 1998 |
| of ribavirin and alpha | Corporation | treatment of chronic hepatitis | December 1998 |
| interferon) | | C in patients with | |
| | | compensated liver disease | |
| | | who have relapsed following | |
| | | alpha-interferon treatment; | |
| | | treatment of chronic hepatitis | |
| | | C in patients with | |
| | | compensated liver disease | |
| | | previously untreated with | |
| | | alpha interferon therapy | |
| Rebif ® | Serono SA and | Relapsing forms of multiple | March 2002 |
| (interferon beta 1-a) | Pfizer Inc. | sclerosis | |
| Recombinate ® rAHF/ | Baxter Healthcare | Blood-clotting factor VIII for the | February 1992 |
| (recombinant | Corporation | treatment of hemophilia A | |
| antihemophilic factor) | (Genetics Institute) | | |
| Recombivax-HB ® | Merck & Company, | Hepatitis B vaccine for | January 1987 |
| (recombinant hepatitis B | Inc. | adolescents and high-risk | January 1987 |
| vaccine) | | infants; adults; dialysis; | January 1989 |
| | | pediatrics | June 1993 |
| ReFacto ® (antihemophilic | Genetics Institute | Control and prevention of | March 2000 |
| factor) | (American Home | hemophilia A and short-term | |
| | Products | prophylaxis to reduce bleeding | |
| | Corporation) | episodes | |
| Refludan ® (lepirudin | Hoechst Marion | For anticoagulation in patients | March 1998 |
| [rDNA] for injection) | Roussel | with heparin- induced | |
| | | thrombocytopenia and | |
| | | associated thromboembolic | |
| | | disease in order to prevent | |
| | | further thromboembolic | |
| | | complications | |
| Regranex ® Gel (gel | Ortho-McNeil and | Platelet-derived growth factor | December 1997 |
| becaplermin) | Chiron Corporation | treatment of diabetic foot | |
| | | ulcers | |
| Remicade ® | Centocor, Inc. | Short-term management of | August 1998 |
| (infliximab) | (subsidiary of | moderately to severely active | November 1999 |
| | Johnson & Johnson) | Crohn's disease including | July 2002 |
| | | those patients with fistulae; | February 2002 |
| | | treatment of patients with | |
| | | rheumatoid arthritis who have | |
| | | had inadequate response to | |
| | | methotrexate alone; Long-term | |
| | | remission-level control of | |
| | | Crohn's disease symptoms; | |
| | | For use in combination with | |
| | | methotrexate in severely | |
| | | active rheumatoid arthritis | |
| Remodulin ™ | United Therapeutics | Pulmonary arterial | May 2002 |
| (treprostinil sodium) | Corp. | hypertension | |
| Renagel ® Capsules | GelTex | Reduction of serum | November 1998 |
| (sevelamer hydrochloride) | Pharmaceuticals, | phosphorus in patients with | July 2000 |
| | Inc. | end-stage renal disease | |
| | | (ESRD); reduction of serum | |
| | | phosphorus in hemodialysis | |
| | | patients with end-stage renal | |
| | | disease | |
| ReoPro ™ (abciximab) | Centocor and Eli | Reduction of acute blood clot- | December 1994 |
| | Lilly & Company | related complications for high- | December 1997 |
| | | risk angioplasty patients; | |
| | | reduction of acute blood clot | |
| | | complications for | |
| | | all patients undergoing any | |
| | | coronary intervention; | |
| | | treatment of unstable angina | |
| | | not responding to conventional | |
| | | medical therapy when | |
| | | percutaneous coronary | |
| | | intervention is planned within | |
| | | 24 hours | |
| RespiGam ® (immune | MedImmune, Inc. | Prevention of respiratory | January 1996 |
| globulin enriched in | | syncytial virus in infants under | |
| antibodies syncytial virus | | 2 with bronchopulmonary | |
| [RSV]) | | dysplasia or history of | |
| | | prematurity against respiratory | |
| Retavase ™ (reteplase | Centocor, Inc. | Management of acute | October 1996 |
| recombinant plasminogen | | myocardial infarction in adults | |
| activator) | | | |
| Rituxan ™ (rituximab) | IDEC | Treatment of relapsed or | November 1997 |
| | Pharmaceuticals | refractory low-grade or | |
| | Corporation and | follicular, CD20-positive B-cell | |
| | Genentech, Inc. | non-Hodgkin's lymphoma | |
| Roche Amplicor HIV-1 | Roche Molecular | In vitro nucleic acid | March 1999 |
| RNA Test | Systems | amplification test used for | |
| | | patient monitoring and as an | |
| | | aid in management of patients | |
| | | on antiviral therapy for HIV | |
| | | disease | |
| Roferon-A ® (recombinant | Hoffmann-La Roche, | Treatment of hairy cell | June 1986 |
| interferon alfa-2a) | Inc. | leukemia; AIDS-related | November 1988 |
| | | Kaposi's sarcoma; chronic | October 1995 |
| | | phase Philadelphia | November 1995 |
| | | chromosome positive chronic | |
| | | myelogenous | |
| | | leukemia; hepatitis C | |
| Saizen ® (recombinant | Serono Laboratories | Treatment of growth hormone | October 1996 |
| human growth hormone) | | deficiency in children | |
| Sarafem ™ (fluoxetine | Interneuron | Treatment of premenstrual | July 2000 |
| hydrochloride) | Pharmaceuticals, | dysphoric disorder | |
| | Inc., and Eli Lilly & | | |
| | Company | | |
| Serostim ® | Serono Laboratories | Treatment of cachexia (AIDS- | August 1996 |
| | | wasting) | |
| Simulect ® | Novartis | Prevention of acute rejection | May 1998 |
| (basiliximab; recombinant | Pharmaceutical | episodes in kidney transplant | March 2001 |
| monoclonal antibody that | Corporation and | recipients; Prevention of | |
| binds the interleukin-2 | Ligand | rejection in combination with | |
| receptor-alpha chain) | Pharmaceuticals, | triple immunosuppressive | |
| | Inc. | therapy in renal transplant; use | |
| | | in pediatric renal transplant; | |
| | | and use of IV bolus injection | |
| SYNAGIS ™ (palivizumab) | MedImmune, Inc. | Prevention of serious lower | June 1998 |
| | | respiratory tract disease | |
| | | caused by respiratory syncytial | |
| | | virus (RSV) in pediatric | |
| | | patients at high risk of RSV | |
| | | disease | |
| Tamiflu ™ (oseltamivir | Gilead Sciences, | Treatment of most common | October 1999 |
| phosphate) | Inc., and Hoffmann- | strains of influenza in adults; | November 2000 |
| | La Roche, Inc. | prevention of influenza in | December 2000 |
| | | adolescents and adults; | |
| | | treatment of acute influenza in | |
| | | children 1 year and older | |
| Targretin ® (bexarotene) | Ligand | Treatment of cutaneous | December 1999 |
| | Pharmaceuticals, | manifestations of cutaneous T- | |
| | Inc. | cell lymphoma in patients who | |
| | | are refractory to at least one | |
| | | prior systemic therapy | |
| Targretin Gel ® | Ligand | Topical treatment of cutaneous | June 2000 |
| (bexarotene) | Pharmaceuticals, | lesions in patients with early- | |
| | Inc. | stage cutaneous T-cell | |
| | | lymphoma | |
| Thyrogen ® (thyrotropin alfa | Genzyme | Adjunctive diagnostic tool for | December 1998 |
| for injection) | | serum thyroglobulin (Tg) | |
| | | testing with or without | |
| | | radioiodine imaging in the | |
| | | follow-up of patients with | |
| | | thyroid cancer | |
| TNKase ™ (tenecteplase) | Genentech, Inc. | Treatment of acute myocardial | June 2000 |
| | | infarction | |
| Tracleer ™ | Actelion Ltd. | Pulmonary arterial | November 2001 |
| (bosentan) | | hypertension with WHO Class | |
| | | III or IV symptoms | |
| TriHIBit ™ | Pasteur Mérieux | Childhood immunization | September 1996 |
| | Connaught | between 15-18 months for | |
| | | acellular pertussis, diphtheria, | |
| | | tetanus and HIB disease | |
| Tripedia ® | Aventis Pasteur | Diphtheria, tetanus and | November 1992 |
| | (formerly Pasteur | acellular pertussis vaccination | July 1996 |
| | Mérieux Connaught) | of infants 2, 4 and 6 months of | August 2000 |
| | | age; first booster at 15-18 | |
| | | months; fifth dose at 4-6 | |
| | | years of age after four doses | |
| Trisenox ™ (arsenic | Cell Therapeutics, | Treatment of acute | September 2000 |
| trioxide) | Inc. | promyelocytic leukemia | |
| Twinrix ® | SmithKline Beecham | Immunization against hepatitis | May 2001 |
| (hepatitis A inactivated and | Biologicals (unit of | A and B viruses | |
| hepatitis B [recombinant] | GlaxoSmith-Kline) | | |
| vaccine) | | | |
| Ultra-sensitive Amplicor | Roche Molecular | Quantitative assay for HIV-1 | March 1999 |
| HIV-1 Monitor Test | Systems | RNA used to assess a | |
| | | patient's prognosis by | |
| | | measuring changes in plasma | |
| | | HIV-RNA levels during the | |
| | | course of antiviral treatment | |
| Venoglobulin-S ® (human | Alpha Therapeutic | Treatment of primary | November 1991 |
| immune globulin | Corporation | immunodeficiencies; idiopathic | January 1995 |
| intravenous 5% and 10% | | thrombocytopenic purpurea | |
| solutions) | | (ITP); Kawasaki disease | |
| Viread ™ | Gilead Sciences | For use in combination with | October 2001 |
| (tenofovir disoproxil | | other antiretroviral agents for | |
| fumarate) | | treatment of HIV-1 infection | |
| VISTIDE ® (cidofovir | Gilead Sciences, | Treatment of cytomegalovirus | June 1996 |
| injection) | Inc. | (CM | |
| | | V) retinitis in AIDS patients | |
| Visudyne ™ | QLT Photo | Treatment of wet form of age- | April 2000 |
| (verteporfin for injection) | Therapeutics and | related macular degeneration; | August 2001 |
| | CIBA Vision | Predominantly classic | |
| | | subfoveal choroidal | |
| | | neovascularization due to | |
| | | pathologic myopia (severe | |
| | | near-sightedness) | |
| Vitravene ™ (fomivirsen | Isis | Treatment of cytomegalovirus | August 1998 |
| sodium, injectable) | Pharmaceuticals, | (CMV) retinitis in patients with | |
| | Inc., and CIBA | AIDS | |
| | Vision | | |
| WelChol ™ (colesevelam | GelTex | Reduction of elevated low- | May 2000 |
| HCI) | Pharmaceuticals, | density lipoprotein (LDL) | |
| | Inc. | cholesterol alone or in | |
| | | combination with HMG-CoA | |
| | | reductase inhibitor (statin) in | |
| | | patients with | |
| | | hypercholesterolemia | |
| Wellferon ® (interferon alfa- | Glaxo Wellcome | Treatment of chronic hepatitis | March 1999 |
| n1, lymphoblastoid) | | C in patients 18 years of age | |
| | | or older without | |
| | | decompensated liver disease | |
| WinRho SDF ® | Nabi | Prevention of Rh | March 1995 |
| | | isoimmunization in pregnant | |
| | | women and the treatment of | |
| | | thrombocytopenic purpurea | |
| | | (TP) (a platelet disorder that | |
| | | can cause uncontrolled | |
| | | bleeding) | |
| Xigris ™ | Eli Lilly and Co. | Severe, life-threatening sepsis | November 2001 |
| (drotrecogin alfa activated; | | | |
| recombinant form of | | | |
| human Activated Protein | | | |
| C) | | | |
| Xyrem ® | Orphan Medical Inc. | Cataplexy associated with | July 2002 |
| (sodium oxybate) | | narcolepsy | |
| Zenapax ® (daclizumab) | Hoffmann-La Roche, | Humanized monoclonal | December 1997 |
| | Inc. | antibody for prevention of | |
| | | kidney transplant rejection | |
| Zevalin ™ | IDEC | Relapsed or refractory low- | February 2002 |
| (ibritumomab tiuxetan) | Pharmaceuticals | grade, follicular, or | |
| | Corp. | transformed B-cell non- | |
| | | Hodgkin's lymphoma | |
| Zonegran ™ (zonisamide) | Elan Corporation | Adjunctive therapy in | March 2000 |
| | | treatment of partial seizures in | |
| | | adults with epilepsy | |

Delivery of Bioactive Compounds

Bioactive compounds may be delivered to an organism by growing individual cells in vitro under conditions that result in the formation of an organized tissue producing the bioactive compound and subsequently implanting the organized tissue into the organism (see above for detailed description of organized tissue production). In certain embodiments, the organized tissue also comprises a subset of cells comprising a DNA sequence encoding a vasculogenic factor or a factor that increases the expression of a vasculogenic factor. In certain embodiments, production of the bioactive compound by the organized tissue is mediated by a DNA sequence present in at least a subset of the cells which make up the implanted tissue.

A variety of bioactive compounds may be delivered by this method, and they may function through intracellular (i.e., within the cells of the organized tissue or organoid), endocrine, autocrine, or paracrine mechanisms). Moreover, the organoid may deliver multiple bioactive compounds either simultaneously or sequentially (e.g., one bioactive compound mediates the delivery of another). Liberation of the bioactive compound from the cells of the organoid may occur by either passive or active processes (e.g., diffusion or secretion). Preferably, the bioactive compound is delivered into the blood stream.

For example, the bioactive compound may be a hormone, growth factor, or the like which is produced and liberated by the cells of the organoid to act locally or systemically on host tissues. Alternatively, the bioactive compound may function within the cells or on the surface of the cells of the organoid to enhance the uptake or metabolism of compounds from the host tissue or circulation (e.g., lactic acid, low density lipoprotein). Where the organoid serves as a functional and structural adjunct to the host tissue, delivery of growth factors by autocrine or paracrine mechanisms may enhance the integration of the organoid into host tissues. Similarly, where multiple bioactive compounds are produced by the organoid, autocrine delivery of one of the bioactive compounds may be used to regulate the production of one or more of the other bioactive compounds. The invention also provides for using insoluble vasculogenic agents which bind up in the local tissues and/or are insoluble and do not get into the bloodstream, for example as demonstrated in Circulation, 2001, Lu et al., 104, incorporated herein by reference in its entirety: 594-599 for VEGF 164/5.

The organoid may be implanted as described below.

In certain embodiments, at least some of the cells of the organoid contain a bioactive compound. In certain embodiments, the production of the bioactive compound is mediated by a DNA sequence encoding the bioactive compound. The DNA sequence encoding the bioactive compound may be extra-chromosomal, integrated into the genomic DNA of the organoid cell, or may result from a mutation in the genomic DNA of the organoid cell. In addition, the cells of the organoid may contain multiple DNA sequences encoding the same or more than one bioactive compound. Moreover, the different cells of the organoid may contain different DNA sequences encoding different bioactive compounds. For example, in one embodiment, a skeletal muscle organoid may include myofibers containing a first DNA sequence encoding a first bioactive compound and fibroblasts containing a second DNA sequence encoding a second bioactive compound. Alternatively, the skeletal muscle organoid could include myoblasts from different cell lines, each cell line expressing a DNA sequence encoding a different bioactive compound. These “mosaic” organoids allow the combined and/or synergistic effects of particular bioactive compounds to be exploited. For example, myoblasts expressing growth hormone may be combined with myoblasts expressing an insulin-like growth factor to produce organoids useful in stimulating muscle growth/regeneration. Similarly, myoblasts expressing a bone morphogenetic protein may be combined with myoblasts expressing a parathyroid hormone to produce organoids useful in stimulating bone and cartilage growth/regeneration.

In a preferred embodiment, the DNA sequence encodes a protein which is the bioactive compound. The protein is produced by the cells and liberated from the organoid, preferably into the bloodstream. Alternatively, the DNA sequence may encode an enzyme which mediates the production of a bioactive compound or a cell surface protein which enhances the uptake and metabolism of compounds from the host tissue or circulation (e.g., lactic acid or low density lipoproteins). The DNA sequence may also encode a DNA binding protein which regulates the transcription of the sequence encoding a bioactive compound or an anti-sense RNA which mediates translation of the niRNA for the bioactive compound. The DNA sequence may also bind trans-acting factors such that the transcription of the sequence (i.e., foreign or native) encoding the bioactive compound is enhanced (e.g., by disinhibition). Furthermore, the DNA sequence may be a cis-acting control element such as a promoter or an enhancer coupled to a native or foreign coding sequence for the bioactive compound or for an enzyme which mediates the production of the bioactive compound.

Implantation

The invention provides for implantation of an organized tissue. An organized tissue can be implanted at any site of an organism, and into any tissue of interest. Implantation can be either subcutaneous (described below and in U.S. Pat. No. 5,869,041) or intramuscular (see U.S. Pat. No. 5,869,041). Implantation according to the invention can be below the skin (subcutaneous), near or around an ischemic area, within ischemic tissues (e.g., in heart muscle), within blood vessels feeding ischemic areas (in coronary arteries or cardiac veins or peripheral arteries or veins), around blood vessels feeding ischemic areas (in coronary arteries or cardiac veins or peripheral arteries or veins). Implantation also includes introducing or placing several organized tissues in or around an ischemic site to create a gradient of angiogenesis. Implantation also includes introducing or placing one or more organized tissues into an artery to 1) stimulate the formation of downstream vessels, for example, capillaries (angiogenesis) or 2) to transform capillaries into arterioles (vasculogenesis).

An implanted organized tissue can also be retrieved or removed from an organism at any time point after implantation, preferably via surgical methods well known in the art or as described in U.S. Pat. No. 5,869,041.

The invention also provides for an implanted organized tissue which is implanted using a trocar, a catheter, an introducer, or a stent, according to methods known in the art.

The organized tissue can also be implanted as follows:

1) the organized tissue is implanted in a highly vascular site of the body (e.g., near or around large blood vessels or vascular networks, or in or near the omentum);

2) the organized tissue is implanted in a site previously or simultaneously treated to stimulate local vascularization (e.g. by using lasers (such as those known in the art for myocardial revascularization), punches or tissue “scoring” with surgical instruments);

3) the organized tissue is implanted with a biomaterial or device (e.g. a degradable polymer or braided silk suture) that stimulates local vascularization; or

4) the organized tissue is comprised of cells (e.g. allogeneic cells) or components (e.g. certain collagens or fibrins) which stimulate a local inflammatory response leading to vascularization.

The organized tissue may be implanted by standard laboratory or surgical techniques at a desired anatomical location within the organism. For example, the organized tissue may be implanted in the same or a different tissue from the tissue of origin of at least one of the individual cells. The location of implantation depends, in part, upon the method of delivery and the identity of the particular bioactive compound to be delivered.

The organoid may be implanted by attachment to a host tissue or as a free floating tissue. In addition, attached organized tissues may be implanted with or without the tissue attachment surfaces used for in vitro tissue formation. Tissues responsive to mechanical forces are preferably implanted by attaching directly to the host tissue or by implanting the organized tissue coupled to the attachment surfaces so that the organized tissue is exposed to mechanical forces in vivo. For example, skeletal muscle organized tissues are preferably implanted by attachment to the host tissue under tension along a longitudinal axis of the organoid. Moreover, the organoids may be permanently or temporarily implanted. Permanent implantation may be preferred, for example, where the organoid produces a bioactive compound which corrects a systemic metabolic error (e.g., delivery of insulin to treat diabetes), whereas temporary implantation may be preferred where only transient delivery of a bioactive compound is desired (e.g., delivery of a growth factor to enhance wound healing). Furthermore, because organoids may be implanted, removed, and maintained in vitro, bioactive compounds may be delivered intermittently to the same or a different location in the organism. For example, a skeletal muscle organized tissue produced from the cells of a human patient (e.g., an autograft) may be implanted at a first anatomical location for a defined period and subsequently implanted at a second location at or after the time of removal.

Cells and Tissues

Cells useful according to the invention include skeletal muscle cells, myoblasts, myofibers, fibroblasts, endothelial cells, smooth muscle cells, cardiac myocytes, osteoblasts, neuronal cells, hepatocytes, mesenchymal stem cells, marrow-derived stem cells, adult stem cells, embryonic stem cells, mesenchymal stem cells, bone marrow derived cells, and bone marrow stromal cells.

Although this method is compatible with the in vitro production of a wide variety of tissues, it is particularly suitable for tissues in which at least a subset of the individual cells are exposed to and impacted by mechanical forces during tissue development, remodeling or normal physiologic function. Examples of such tissues include muscle, bone, skin, nerve, tendon, cartilage, connective tissue, endothelial tissue, epithelial tissue, and lung. More specific examples include skeletal, cardiac, and smooth muscle, stratified or lamellar bone, and hyaline cartilage. This method is also compatible with the in vitro production of adipose tissue, and tissues comprising either mesenchymal stem cells, bone marrow derived cells, bone marrow stromal cells and neural connective tissue. Where the tissue includes a plurality of cell types, the different types of cells may be obtained from the same or different organisms, the same or different species, the same or different donors, and the same or different tissues. The cells of the organized tissue may be allogeneic or autologous. Moreover, the cells may be primary cells or immortalized cells. Furthermore, all or some of the cells of the tissue may contain a nucleic acid sequence which mediates the production of a bioactive compound (as described herein).

The invention also provides for organized tissues comprising xenogenic cells (for example porcine cells) that have been humanizing according to methods known in the art.

Assays for Vascularization

Vascularization of an organized tissue of the invention can be measured by any of the following methods:

1. Assay for increased blood vessel density by measuring endothelial cell number or proliferation

An assay to detect changes in capillary density in an organized tissue of the invention is performed as follows.

BAM and host muscle explants are either frozen in isopentane or fixed with 0.25% glutaraldehyde for cryostat sectioning. Capillary density is examined by quantitation of endothelial cells in cryostat sections stained with anti-mouse CD31 (Pharmingen), an antibody specific for mouse endothelial cells, following standard immunoperoxidase procedures and development with DAB. The primary antibody is omitted from negative controls. Five non-overlapping microscopic fields are analyzed from each explanted BAM using Zeiss KS 300 Version 3.0 Image Analysis System, and the area staining positive for CD31 is quantitated and expressed as a percentage of the total area analyzed.

2. Increased blood flow assayed with doppler, MRI or ultrasound (Schratzberger et al., 2001, J. Clin. Invest., 107:1215-8; Konstam et al., 1991, 10(5 Pt 1), 750-6; Couffinhal et al., 1999, Circulation, 99:318898).

3. Imaging for endothelial cells or smooth muscle cells with histology, microscopy, microsphere beads, or immunohistochemistry, (Lu, et al., 2001, Circulation, supra):

4. Assessment of skin color; warmth and presence or absence of pulse;

5. Treadmill testing. 6. Microbead Assays (Cao et al., 1998, Lab Invest, 78:1029-30).

Additional assays for Vascularization are also known in the art.

Treatment of Disease

The invention provides a method of treating a disease in an organism comprising delivering a bioactive compound and/or a vasculogenic factor to an organism by an organized tissue construct that is vascularized following implantation.

The method of the invention can be used to treat a disease including but not limited to blood disorders, ischemic disease, bone or joint disorders, cancer, cardiovascular disorders, endocrine disorders, immune disorders, infectious diseases, muscle wasting and whole body wasting disease,

Ischemic Disease

Coronary and peripheral artherosclerotic diseases often result in ischemic disorders in the heart and limbs. Patients with severe or diffuse arterial disease are not candidates for existing operative and percutaneous revascularization techniques. Therapeutic angiogenesis by the administration of growth factors known to induce neovascularization represents an innovative approach for the treatment of ischemic disease. Vascular endothelial growth factor (VEGF) is one of the most potent and specific vasculogenic growth factors currently known. VEGF gene transfer has been used to induce collateral blood vessel development and preserve blood flow to ischemic tissues in animal models of hind limb (Bauters, C. et al., 1995, Circulation, 91: 2802; Cheleboun, J.et al., 1994, Aust N Z J Surg., 64: 202) and cardiac ischemia (Mack, C. et al., 1998, J Thorac Cardiovasc Surg., 115:168). Phase I and II trials are currently underway to test the effects of VEGF in patients with critical limb ischemia (Baumgartner, I. et al., 1998, Circulation, 97: 1114) and severe coronary artery disease (Losordo, D. et al., 1998, Circulation, 98: 2800; Rosengart, T. et al., 1999, Circulation, 100: 468) with preliminary results that appear promising in the short term (Ylä-Herttuala, S. et al., 2000, Lancet, 355: 213; Vale P., et al., 2000, Circulation, 102: 965). Achieving the appropriate dosing and pharmacokinetics of vasculogenic factors will be critical to the long term safety and success of these procedures and is likely to vary from disease to disease.

The invention provides for genetically engineered myoblasts that are tissue-engineered ex vivo into BioArtificial muscles (BAMs). When implanted in nonmuscle or muscle sites, they survive long-term and deliver predictable levels of gene products such as growth hormone, insulin-like growth factors and erythropoeitin for months (Vandenburgh, H. et al., 1996, Hum Gene Ther. 7: 2195; Vandenburgh, H., 1998, Hum Gene Ther, 9: 2555). BAMs genetically modified to secrete rVEGF can also stimulate localized angiogenesis in a predictable dose-dependent manner. (See Examples below). Human BAMs delivering vasculogenic or arteriogenic factors could be useful in treating a range of cardiovascular diseases and ischemic diseases including but not limited to ischemic heart disease, chronic heart failure, peripheral artery disease, neuropathy, wound healing (soft and hard tissue).

The invention also relates to using a vascularized organized tissue of the invention for treatment of a disease including but not limited to the diseases listed below.

A. Blood Disorders

The invention provides methods of treating blood disorders, including anemia, hemophilia, thrombocytopenia and neutropenia.

Several blood disorders have been treated successfully by the delivery of recombinant human proteins. These disorders include hemophilia, which has been treated by delivery of factor IX (Yao, et al., 1992, Proc. Natl. Acad. Sci. 89, 3357-3361), a plasma glycoprotein essential for blood coagulation, and neutropenia, which has been treated with granulocyte colony stimulating factor (Dale et al., 1993, Blood 81, 2496-2502) which promotes growth, differentiation and functional activity of neutrophils. Anemia has been successfully treated with erythropoietin (EPO) (Hamamori et al., 1994, Hum. Gene. Ther. 5, 1349-1356), the primary regulator of mammalian red blood cell production.

Hemophilia

Hemophilia is an X chromosome-linked recessive bleeding disorder resulting from decreased levels of either factor VIII, factor IX or factor XI (all of which are needed for normal blood coagulation) caused by a genetic abnormality. Hemophiliacs are at risk for bleeding after dental work, surgery, and trauma, and may also suffer internal bleeding with no apparent cause. The most common type of hemophilia (hemophilia A) is a disorder of the intrinsic pathway for the formation of thrombin resulting from a reduction in the coagulant titer of antihemophilic factor (factor VIII:C). Antihemophilic factor is a component of the factor VIII/vWF complex that is regulated by a variety of factors including exercise and hormones; the amino acid sequences necessary for blood coagulation are contained within factor VIII:C.

Hemophilia affects only males who, in turn, pass the abnormal gene onto their daughters, all of whom are carriers. Although women who carry the gene are typically asymptomatic, female carriers can frequently be detected due to the presence of a decreased concentration of factor VIII:C in the plasma, as compared to vWF (Berne and Levy et al., supra). Many individuals with hemophilia die early in life as a result of severe bleeding. However, hemophilia can be treated by transfusion with normal plasma thereby supplying the missing clotting factors and allowing clotting to occur normally on a temporaly basis. Although treatment with purified clotting factor (e.g. factor VIII:C) can be used prophylactically to prevent episodes of bleeding (Berne and Levy et al., supra, Guyton, 1985, Anatomy and Physiology, Saunders College Publishing, Philadelphia) because the infused clotting factor remains active for only a short time, serious bleeds may require repeated infusions to stop the bleeding. Often people with severe hemophilia will be treated with prophylactic clotting factor infisions on a regular basis to avoid bleeding episodes.

Treatment of hemophilia by delivery of recombinant human clotting factors would avoid the risk of contamination by human blood-borne viruses, as well as the necessity for frequent infusion treatments. Recently animal models have been developed for the delivery of recombinant human clotting factors. Using a mouse model for severe hemophilia A, donor bone marrow cells were genetically modified to secrete recombinant human factor VIII (GeneBank Accession #119767) and transplanted into hemophiliac mouse recipients (Evans et al., 1998, Proc. Natl. Acad. Sci. USA, 95: 5734-5739). In a second model, C2C12 myoblasts were genetically modified to secrete biologically active factor IX (GeneBank Accession #439774) and injected into the leg muscles of C3H mice, resulting in factor IX expression in the serum (Yao et al., Proc. Natl. Acad. Sci. USA, 89: 3357-3361).

Neutropenia

Neutropenia, a deficiency in circulating neutrophils, leads to a susceptibility to recurrent and often life-threatening infections. Types of neutropenia include chronic congenital, and cyclic, the latter being characterized by regular oscillations in blood neutrophil counts. Neutropenic individuals generally are asymptomatic until the occurrence of an infection. If the neutrophil count decreases to less than 1000 cells per .pl, there can be an increase in the risk of infection. A neutrophil count of less than 500 cells per ill can be life threatening. Neutropenia can be caused by a variety of factors including decreased production in the bone marrow, increased destruction of neutrophils in the periphery, or an increase in the rate of neutrophil loss to the tissues. A decrease in neutrophil production can result from a particular disease (e.g. aplastic anemia, or leukemia) or from suppression by a toxic drug or irradiation. Cancer chemotherapy, which kills neutrophils in the bone marrow, is also a cause of neutropenia, and patients with advanced HIV infection frequently have severe neutropenia.

Treatment of neutropenia includes antibiotics to fight infections, and more recently, the injection of G-CSF or GM-CSF to promote the growth, differentiation, and functional activity of cells of the neutrophil lineage (Andreoli et al., 1997, Cecil Essentials of Medicine, Fourth Edition, W. B. Saunders Company, Philadelphia and Berkow et al., editors, 1997, The Merk Manual of Medical Information, Merck Research Laboratories, New Jersey). Recombinant human G-CSF injected into neutropenic patients has been shown to increase neutrophil counts by about 16-fold (Dale et al., 1993, Blood, 81: 2496-2502). In an animal model, primary myoblasts isolated from neonatal Fisher rats were genetically engineered to secrete the human G-CSF gene and injected into the gastrocnemius muscle of adult rats (Bonhamn et al., 1996, Hum. Gene Ther., 7:1423-1429). Absolute neutrophil counts of rats receiving the transduced myoblasts were significantly increased up to 15 fold following transplantation, while rats implanted with control myoblasts showed no increase in neutrophil counts.

Anemia

Anemia refers to a decrease in the circulating mass of red blood cells (erythrocytes) resulting from decreased production, premature destruction or loss due to hemorrhage. Furthermore, anemia is a symptom of end-stage renal failure. A decrease in erythrocyte synthesis can result from i. hypocellularity of the bone marrow, ii. replacement of the bone marrow by tumor tissue, iii. suppression of hematopoiesis (e.g. during renal failure, or from a vitamin B 12 or folic acid deficiency) or iv. from a deficiency in iron necessary for the formation of heme. A number of factors including hereditary defects in the red blood cell outer membrane, or direct chemical, physical or immunological injury can cause premature destruction of erythrocytes. The most common form of anemia in Western countries is iron-deficiency anemia resulting from either blood loss or the use of iron by the fetus during pregnancy (Berne and Levy eds., 1993, Physiology, Mosby Year Book, St. Louis).

The pathogenesis of a particular form of anemia dictates the method of treatment. For 25 example, iron-deficiency anemia may be treated with iron, pernicious anemia may be treated with vitamin B 12, while other forms of anemia may be treated with either red cell replacement or erythropoietin (Berne and Levy, supra).

Erythropoietin (EPO), a 3OkD glycoprotein that functions as the primary regulator of mammalian red blood cell production, increases erythrocyte production by stimulating the proliferation, and preventing the apoptosis of erythroid precursors. Anemia related to diminished red blood cell production in patients with end-stage renal failure has been successfully treated with direct tri-weekly injections of recombinant human erythropoietin (GeneBank Accession #182198, Evans, 1991, Am. J. Kidney Dis., 18: 62-70). However, this method of treatment is expensive and is not the most physiological delivery procedure. Several animal models have been developed for delivery of sufficient quantities of EPO to sustain therapeutic erythropoiesis. These include a gene transfer system in which mouse myoblasts genetically modified to secrete human EPO are injected into the skeletal muscles of mice (Hamamori et al., 1994, Hum. Gene. Ther., 5:1349-1356), and a system wherein autologous smooth muscle cells engineered to secrete rat EPO are infused into the carotid artery of Fisher rats (Osborne et al., 1995, Proc. Natl. Acad. Sci., USA, 92:8055-8058). In both studies, hematocrits were significantly increased by the delivery of recombinant EPO.

Thrombocytopenia

Thrombocytopenia refers to a deficiency in the numbers of platelets in the circulating blood. Because thrombocytopenia is commonly caused by platelet specific antibodies that attack and destroy platelets it is considered an autoimmune disease. Other less common causes of this disease include poisoning by toxins or drugs. In cancer patients thrombocytopenia is caused by impaired platelet production from the bone marrow resulting from chemotherapy or radiation treatment. Thrombopoietin (TPO, Genbank Accession #235118) is the primary regulator of megakaryocyte and platelet production. Animal models have been developed for TPO knockout mice, which have a 90% reduction in platelet counts (Mutone et al., 1998, Stem Cells, 16:1). Recently, thrombocytopenic patients have been treated with recombinant human interleukin-I 1 (rhIL-11, Genbank Accession #186273; Neumega, Genetics Institute Inc., Cambridge Mass.), a novel thrombopoietic growth factor (Issacs et al., 1997, J. Clin. Oncol., 3368). The potential exists for the delivery of both thrombopoietin and IL-11 for the treatment of thrombocytopenia from organized tissue constructs.

A common symptomatic manifestation of thrombocytopenia is a large number of minute hemorrhages located in the skin and in the deep tissue that eventually cause purplish discolorations over the surface of the body. These hemorrhages result from an inability of the platelets to stop small bleeding points in the vasculature. Although the hemorrhages can be temporarily inhibited by transfusion with either fresh whole blood or separated platelets, both procedures can be difficult to perform (Guyton et al., supra).

B. Bone or Joint Disorders

The invention provides methods of treating bone or joint disorders, including osteoporosis and osteoarthritis.

Osteoarthritis

Osteoarthritis (also known as degenerative arthritis or degenerative joint disease) is an age-related, chronic disorder of the joints that is associated with degeneration of joint cartilage and formation of new bone at the joint surfaces, often causing pain and stiffness. A variety of biological and mechanical factors can result in osteoarthritis. Osteoarthritis can generally be classified as primary (associated with aging) or secondary (associated with a well-defined cause e.g. inflammatory or connective tissue disease).

Numerous pathologic changes including cartilage fibrillation, fissuring, and erosion (leading to bare areas of bone), spur formation at joint margins, and sclerosis and thickening of subchondral bone are associated with osteoarthritis. The major symptoms of osteoarthritis include progressive pain and stiffness in the joints (most typically hips, knees, spine and small joints of the hands and feet). Other symptoms may include cracking of the joint, deformity due to joint enlargement, and limitation of motion.

Methods of treatment of osteoarthritis may include appropriate forms of exercise, supports or braces, physical therapy, surgery and the administration of analgesics or nonsteroidal anti-inflammatory drugs to reduce pain and swelling (Andreoli et al., 1997, supra and Berkow et al., supra). Transforming growth factor beta (TGF-beta.) has powerful modulatory effects on the skeletal system, enhancing bone formation and decreasing matrix degradation, thus playing a part in the maintenance of bone mass (Boonen et al., 1997, J. Internal Med., 242:285-290). It has been suggested that interleukin-1 receptor antagonist, as well as other recombinant proteins, may be potentially usefuil for preventing and treating osteoporosis by stimulating bone formation (Evans et al., 1998, Ann. Rheum. Dis., 57:125).

Mice that are aged 7 months and older develop spontaneous osteoarthritic lesions in the mandibular condyle cartilage of the temporomandibular joint, and thereby provide an art-accepted model for studying cartilage loss associated with osteoarthritis (Livne et al., 1985, Arthritis and Rheumatology, 28:1027-1038).

Osteoporosis

Osteoporosis, the most common form of metabolic bone disease, is characterized by a reduction in bone mineral and bone matrix that produces bone that is of a normal composition but is decreased in density and is therefore more likely to fracture. Typically, osteoporosis results from the normal effects of menopause in women, and aging, in both men and women. However, other disorders including glucocorticoid excess, hypogonadism, hyperthyroidism, hyperparathyroidism, vitamin D deficiency, gastrointestinal diseases, bone marrow disorders, immobilization, connective tissue diseases and certain drugs can cause osteoporosis.

In the absence of the occurrence of a fracture, osteoporosis is asymptomatic. Following the occurrence of bone collapse or fracture, bone pain may occur and deformities may develop. The most common types of fractures in patients with osteoporosis are vertebral compression fractures or fractures of the wrist, hip, pelvis or humerus. Osteoporosis can be diagnosed prior to the occurrence of a fracture by a variety of methods that measure bone density. These measurements can also be used to predict the development of certain osteoporotic fractures.

Although presently, established osteoporosis cannot be reversed, methods of early intervention can prevent osteoporosis in most individuals, and later intervention can inhibit the progression of the disease. Methods of treatment of osteoporosis include increasing dietary calcium (calcium can slow but not prevent bone loss in women in the early stages of menopause), estrogen treatment (estrogen replacement therapy prevents bone loss in estrogen deficient women), calcitonin treatment (calcitonin appears to prevent loss of bone in the spine of women in either the early or late stages of menopause without affecting appendicular bone loss), biophosphonates (biophosphonates inhibit resorption of osteoclastic bone) and vitamin D and its metabolites (Andreoli et al., supra and Berkow et al., supra).

Recombinant proteins can be useful for attenuating osteoporosis. Bone morphogenetic protein (BMP) is a family of bioactive factors that stimulate new bone formation in ectopic sites by inducing the differentiation of primitive mesenchymal cells into bone producing cells (Strates et al., 1988, Am. J Med. Sci., 296:266-269). Therefore, recombinant human bone morphogenetic protein (rhBMP) may be useful for the treatment of osteoporosis (Urist et al., 1985, Progress in Clinical and Biological Research, 187:77-96). Growth hormone (GH) has been thought to augment bone turnover, increase bone formation and, to a lesser extent, increase bone resorption (lnzucchi et al., 1994, J. Clinical Endocrinol. Metab., 79: 691-694). GH replacement therapy may be a useful method of treating osteoporosis. Insulin-like growth factor-I (IGF-I) enhances cartilage and bone formation, and decreases matrix degradation, thereby indicating that it is an important stimulator of skeletal growth and is relevant to the maintenance of bone mass (Schmid, 1993, J. Int. Med., 234: 535-542). IGF-I replacement therapy may be useful for treatment of osteoporosis. Platelet-derived growth factor-BB (PDGF-BB) is one of the many systemic factors involved in the bone formation cascade at sites of bone resorption (Watrous et al., 1989, Seminars in Arthritis and Rheumatology, 19: 45-65). Therefore, recombinant human platelet-derived growth factor (rhPDGF-BB) may be useful for stimulating bone formation in the prevention and treatment of osteoporosis (Watrous et al., supra).

Although parathyroid hormone (PTH) had initially been thought to be a catabolic agent to the skeletal system, recent evidence has suggested that PTH exerts a direct inhibitory effect on bone resorption and an indirect stimulatory effect on bone resorption mediated by osteoblasts (Dempster et al., 1993, Endocrine Review, 14:690-709). Therefore, recombinant human parathyroid hormone (rhPTH) may be useful for the treatment of osteoporosis (Reeve, 1996, J. Bone and Mineral Research, 11:440-445).

TGF-beta. has powerful modulatory effects on the skeletal system, enhancing bone formation and decreasing matrix degradation, thus playing a part in the maintenance of bone mass (Boonen et al., supra). Therefore, recombinant human TGF-beta. may be a useful drug for stimulating bone formation in the prevention and treatment of osteoporosis (Boonen et al., supra).

Several animal models have been useful for studies of osteoporosis, most notably the ovariectomized (OVX) rat. OVX rats display significantly decreased trabecular bone volume (41%) and decreased mechanical strength of the femoral neck (15.8%) (Peng et al., 1994, Bone, 15:523-532).

C. Cancer

The invention also provides methods of treating cancer.

Cancer is a disease that is characterized by uncontrolled growth of abnormal or cancerous cells, in most instances as a result of an altered genome in a single abnormal cell. The alteration in the genome is caused by a mutation in one or more genes wherein the probability of the occurrence of a mutation is increased by a variety of factors including i. ionizing radiation, ii. exposure to chemical substances known as carcinogens, iii. some viruses, iv. physical irritation, and v. hereditary predisposition. It is thought that a single mutation is insufficient to convert a normal cell into a cancer cell, and that cancer is caused by several independent genetic alterations (Guyton, supra, Alberts et al., 1994, Molecular Biology of the Cell, Garland Publishing, Inc., New York).

Neoplasms including solid tumors such as malignant melanoma, and blood-borne cancers such as leukemia, arise from normal cell populations which have lost the ability to adequately respond to either intracellular or extracellular growth controlling mechanisms. Furthermore, cancer cells are less adherent to each other, as compared to normal cells. As a result, these abnormal cell populations divide at a more rapid rate than their normal cellular counterparts and, in the case of solid tumors, are capable of invading adjacent tissue. Cancerous cells enter the blood stream, migrate to distant sites within the body and eventually colonize secondary organs, a process known as metastasizing. Much of the damage of cancer cells results from the overuse of nutrients by cancer cells (due to the fact that they proliferate indefinitely) as compared to normal cells.

Cancers are classified according to the tissue and cell type from which they are derived and each type of cancer demonstrates characteristics that reflect the cell type of origin. In general, cancers that originate from different cell types are associated with different diseases (Guyton, supra, Alberts et al., supra).

Several therapeutic approaches have been used to slow the progression of dividing tumors. En bloc resection of the primary tumor followed by radiation therapy, chemotherapy or a combination of the two are conventional methods employed to treat the vast majority of tumor types. These modalities, however, can be ineffective and potentially harmful. The site of the tumor, surgical complications such as hemorrhage and the inability to locate tumor masses in a diseased organ can hinder potentially effective operative procedures. In addition, radiotherapy and chemotherapy are associated with ionizing damage of healthy tissue and systemic toxicity respectively.

Alternative approaches to the conventional treatments described above may include the delivery of recombinant molecules which function to either boost the host's immune response to invading metastases or to either directly or indirectly suppress cancerous cell growth. Such molecules may include various cytokines such as interleukin-2 (IL-2), granulocyte-macrophage colony stimulating factor (GM-CSF), interleukin-12 (IL-12) and interferon-gamma (IFN-gamma), anti-angiogenic molecules and tumor associated antigens (Anderson, et al., 1990, Cancer Res., 50: 1853, Stoklosa, et al., 1998, Ann Oncol., 9:63, Leibson, H. J. et al., 1984, Nature, 309:799, Book, et al., 1998, Semin. Oncol. 1998, 25:381, Salgaller, et al., 1998, J. Surg. Oncol., 68: 122, Griscelli, et al., 1998, Proc. Natl. Acad. Sci. USA, 95: 6367).

D. Cardiovascular Disorders

The invention also provides methods of treating cardiovascular disorders, including vascular disease, coronary artery disease and congestive heart failure.

Vascular Disease

Vascular disease is a disease related to poor circulation, that is a common complication in patients who have had atherosclerosis or diabetes for a prolonged period of time. Peripheral vascular disease results from hardening, narrowing, or closing off of both the larger and smaller blood vessels in the limbs (commonly the legs), causing foot sores, ulcers, or gangrene. Severe cases of peripheral vascular disease require amputation of the infected limb. Cardiac vascular disease is caused by poor circulation in the heart muscle (often resulting from a heart attack), leading to defective pumping of the heart. If diagnosed early, vascular diseases may be treatable with angiogenic recombinant proteins, such as VEGF (Mack et al., 1998, J. Vase. Surg., 27:699-709) and/or members of the FGF family (elillo et al, 1997, Circ Res. 35:80-489). In a rodent (rat) model of peripheral disease, the left common femoral artery is ligated and divided in a hindlimb resulting in ischemia (Mack et al., supra). A similar rodent heart model has been developed wherein myocardial infarction is induced by ligating a coronary artery (Yang et al., 1995, Circulation, 92:262-267). As a result of this procedure vascularity and blood flow are reduced in the affected tissue.

Congestive Heart Failure

Congestive heart failure is a disease related to the inability of the heart to function as an efficient pump. Congestive heart failure is a multiple-etiology disorder, that can result from cardiomyopathy, myocardial infarction, or coronary insufficiency (Yang et al., supra). This disorder is characterized by a decrease in stroke volume and cardiac output. Current treatments for this disease, such as digitalis and angiotensin-converting enzyme inhibitor, can improve the condition of the heart, but do not effectively treat the symptoms of pain and exercise intolerance. A rodent (rat) model of congestive heart failure has been developed wherein myocardial infarction is induced by ligating the left coronary artery (Yang et al., supra). Previous studies have shown that systemic administration of rhGH and/or rhIGF-1 can improve the symptoms of congestive heart failure and improve cardiac performance (Yang et al., supra, Stromer et al., 1996, Circ. Res., 79:227-236).

Coronary Artery Disease

The accumulation of fatty deposits in the cells that line the wall of the coronary artery leading to the obstruction of blood flow, is known as coronary artery disease. As a result of coronary artery obstruction, cardiac ischemia (insufficient blood flow) leading to heart damage can occur. Cardiac ischemia is most commonly caused by coronary artery disease. Angina and heart attack are the major complications of coronary artery disease. Treatment of angina includes administration of beta-blockers, nitrates, calcium antagonists and antiplatelet drugs and, in some cases, angioplasty. Treatment of heart attacks includes reducing the clot in the coronary artery (e.g. by aspirin treatment, thrombolytic therapy, angioplasty or coronary artery bypass surgery) (Andreoli et al., supra and Berkow et al., supra). A method of treatment of coronary artery disease may involve administration of angiogenic proteins such as VEGF (Mack et al., supra) and/or members of the FGF family (Melillo et al., supra).

Cardiomyopathy

The term cardiomyopathy refers to a group of diseases (dilated, hypertrophic and restrictive cardiomyopathy) effecting the heart muscle. Dilated cardiomyopathy is associated with dilation of one or both ventricles of the heart and impaired systolic function. The enlarged ventricles are unable to pump a sufficient amount of blood to the body and as a result, heart failure occurs. The most common cause of dilated cardiomyopathy is coronary artery disease. The symptoms of dilated cardiomyopathy include shortness of breath, increased heart rate, fluid retention in the legs and abdomen, fluid uptake by the lungs, heart murmurs and abnormal heart rhythms. The method of treatment depends on the underlying cause of the dilated cardiomyopathy and may include administration of nitrate, beta-blockers or calcium channel blockers (for individuals with coronary artery disease), administration of anticoagulants to prevent clots, administration of agents that reduce the force of heart contractions or prevent abnormal heart rhythms, treatment with diuretics or administration of digoxin.

Hypertrophic cardiomyopathy is a disease associated with a thickening of the ventricular walls. This condition may be the result of a birth defect, or may occur in individuals with acromegaly or pheochromocytoma. As a result of thickened ventricular walls, there is increased resistance in the heart to blood flowing from the lungs. Consequently, as back pressure develops in the lung veins, fluid accumulates in the lungs causing shortness of breath. The symptoms of hypertrophic cardiomyopathy include faintness, chest pain, palpitations (resulting from irregular heartbeats) and heart failure with shortness of breath. Hypertrophic cardiomyopathy is most commonly treated with beta-blockers or calcium channel blockers.

Restrictive cardiomyopathy refers to disorders wherein the ventricular walls stiffen without thickening, and resist the normal pattern of filling with blood that occurs between heartbeats. When the heart is only partially filled with blood, an inadequate amount of blood can be pumped to an individual engaged in exercise. In one form of restrictive cardiomyopathy a gradual replacement of the heart muscle by scar tissue occurs. The other form of restrictive cardiomyopathy is characterized by infiltration of the heart muscle by material such as white blood cells, not normally found in the heart. The symptoms commonly associated with restrictive cardiomyopathy include heart failure with shortness of breath, tissue swelling (edema), abnormal heart rhythms and palpitations. Restrictive cardiomyopathy can be treated by administering diuretics or by treating the underlying cause of this disorder (Andreoli et al., supra and Berkow et al., supra). A method of treatment of cardiomyopathy may involve administration of GH or inotropic agents (Lombardi et al., 1997, Horm. Res., 48:38 and Cittadini et al., 1997, Endocrin., 138: 5161).

E. Endocrine Disorders

The invention provides methods of treating endocrine disorders, including diabetes, obesity and growth hormone deficiencies.

Diabetes

Diabetes mellitus is a heterogenous group of four diseases (type I and II diabetes, gestational diabetes and diabetes secondary to other conditions) characterized by high levels of blood glucose resulting from defects in insulin secretion, insulin action, or both. The four different classes of diabetes are thought to have different etiologies but similar pathologic courses following the onset of diabetes.

Insulin dependent or type I diabetes results from an insulin deficiency caused by beta.-cell destruction. As a result of a decrease in the level of insulin and a concomitant increase in the level of glucagon, there is an increase in glucose production in individuals with type I diabetes. Due to a reduction in the efficiency of peripheral glucose use, plasma glucose levels are increased. As glucose utilization goes down, fat utilization is increased thereby resulting in increased levels of keto acids in the extracellular fluids. The symptoms of type I diabetes include glucose excretion in the urine accompanied by increased excretion of water and salts and frequent urination, increased thirst, changes in catabolism leading to loss of lean body mass, adipose tissue and body fluids, deficits in various intracellular components, and abnormalities of the eye. Treatment of this form of diabetes with insulin results in decreased levels of plasma glucose, free fatty acids, and ketoacids and a reduction in urine nitrogen losses.

Noninsulin-dependent or type 2 diabetes is the most common form of diabetes mellitus and is characterized by impaired insulin-mediated glucose uptake or insulin resistance by the major target tissues. Type II diabetes is frequently associated with obesity. The major symptom of type II diabetes is an elevated fasting level of plasma glucose due to overproduction of hepatic glucose. Treatment of type II diabetes can include caloric regulation, weight reduction if the disease is accompanied by obesity, and the administration of sulfonylurea drugs to improve both tissue responsiveness to endogenous insulin and beta.-cell responsiveness to glucose. Insulin injections are required for treating the late stages of the disease (Beme and Levy et al., supra). Leptin may also be useful for the treatment of diabetes via regulation of the levels of blood glucose and fat (Murphy et al., 1997, Proc. Natl. Acad. Sci. USA, 94:13921).

Obesity

Obesity is defined as an accumulation of excessive body fat. Individuals are considered obese if their weight is 20% or more over the midpoint of their weight range according to a standard height-weight table. Obesity occurs when the consumption of calories exceeds calorie usage by the body. Mechanistically, obesity is caused either by a failure of adipose cells to send signals to the brain (thereby regulating food seeking and consumption behavior) or failure of the brain to respond to signals from adipose tissue in an appropriate manner. To a large degree obesity is genetically predetermined.

Obese individuals may experience poorly regulated glucose in the blood, breathing difficulties, shortness of breath and severe respiratory problems resulting from pressure being exerted on the lungs from excess fat accumulated below the diaphragm and in the wall of the chest. Kidney problems, orthopedic problems, skin disorders and edema may also be associated with obesity. Methods of treatment of obesity include severely decreased caloric intake and surgery to reduce stomach size (Andreoli et al., supra and Berkow et al., supra). Obesity may also be successfully treated by regulating the levels of blood glucose and fat with leptin and/or insulin. The genetically obese mouse represents an animal model for diabetes and obesity (Murphy et al., 1997, Proc. Natl. Acad. Sci USA, 94: 13921-13926).

Growth Hormone Insufficiency

Growth hormone is a single-chain protein with a molecular weight of 22,000 that is normally produced by a pituitary gene. The synthesis of growth hormone is regulated by growth hormone releasing hormone, thyroid hormone and cortisol. Growth hormone secretion can be stimulated by a variety of factors (e.g. a decrease in the levels of glucose or fatty acids, fasting, exercise or estrogens), and inhibited by various factors (e.g. somatostatin, an increase in the level of glucose or fatty acids, or growth hormone).

A number of mechanisms including hypothalamic dysfunction, pituitary tumors, an inactive growth hormone protein, decreased production of peptide hormone mediators of growth hormone action (e.g. somatomedins) or receptor abnormalities, can result in a growth hormone deficiency in children. The physiological manifestations of a growth hormone deficit in children include short stature (for example Turner's Syndrome), delayed bone maturation, mild obesity, and delayed puberty. Tumer's Syndrome is a gonadal disorder affecting females in which their is partial or total loss of one of the X-chromosomes. This disease is characterized by short stature, and various somatic anomalies including epicanthal folds, low-set ears, webbed neck, multiple pigmented nevi, lymphedema of the hands and feet, renal malformations and coarctation of the aorta (Andreoli et al., supra and Berkow et al., supra). Treatment with growth hormone can result in increased nitrogen retention, increased lean body mass, decreased adipose mass, increased growth speed (in children), the initiation of puberty and the establishment of fertility (Berne and Levy, supra).

Dwarfism can be caused by a decrease in growth hormone secretion that is most commonly due to a hereditary defect. Another less common form of dwarfism is caused by a failure of the anterior pituitary gland to secrete growth hormone. The physical characteristics of a pituitary dwarf include a failure to demonstrate normal organ and bone growth, repressed sexual development, and short stature (Guyton, supra). Dwarfism in humans results in many instances from reduced growth hormone (GH) secretion from the brain's pituitary gland (Daughaday et al., 1995, In Growth Hormone, Harvey et al., eds., CRC Press Inc., Boca Raton, 475-504). In an animal model of this disease, growth deficient rats (dwarf DW4 rats) are approximately 40% smaller than age-matched normal rats due to expression of pituitary GH at levels that are 5-10% of normal (Charlton et al., 1988, J. Endocrinol., 119: 51-58).

F. Imumune Disorders

The invention provides a method of treating immune disorders including Chronic granulomatous disease (CGD), acute/chronic renal failure, severe combined immunodeficiency and autolnmmune disorders. The invention also provides a method of delivering a composition useful for vaccination (e.g. against whooping cough).

Chronic Granulomatous Disease

CGD is a recessive disorder characterized by a defective phagocyte respiratory burst oxidase, life-threatening pyogenic infections and inflammatory granulomas (Pollock et al., 1995, National Genetics, 9:202-209). Methods of treating CGD with recombinant proteins such as gamma interferon are designed to maintain a constant level of recombinant protein in the bloodstream. In one animal model of this disease, Mycobacterium marinum caused CGD in immunocompetent leopard frogs (Rana pipiens) (Ramakrishnan et al., 1997, Infectious Immunology, 65:767-773). Another animal model for CGD is a knock out mouse wherein a mouse contains a null allele of a gene involved in X-linked CG (the 91 kD subunit of oxidase cytochrome b) (Pollock et al., supra).

Acute or Chronic Renal Failure

Kidney failure is defined as an inability of the kidney to filter blood and excrete toxic substances from the body. Acute kidney failure refers to a rapid loss of kidney function and is often associated with multiple organ failure and sudden death. Chronic kidney failure is defined as a gradual and progressive deterioration of kidney function often associated with diabetes and high blood pressure.

The rapid decline in the ability of the kidney to remove toxic substances from the blood that occurs during acute kidney failure, results in an increase in the level of nitrogenous waste products (e.g. urea) in the blood. Acute kidney failure can be caused by any condition that i. results in a reduction in the flow of blood to the kidney, ii. interferes with the flow of urine after it has left the kidneys, or iii. produces an injury to the kidney. The symptoms associated with acute kidney failure are variable and depend on the initial cause of kidney damage. Often, a condition that results in acute renal failure may produce symptoms unrelated to the kidneys, including high fever, shock and heart failure. Symptoms of acute renal failure resulting from an obstruction of urine flow may include cramping, resulting from stretching of the urine collecting area, and blood in the urine. Decreased urine output, as well as increased levels of creatinine, urea, acid, potassium and decreased sodium in the blood, can be indicative of acute kidney failure. Acute kidney failure can be successfully treated by restricting water intake, administration of particular amino acids to maintain a sufficient protein level, restricting the uptake of substances that are eliminated through the kidney, administration of antacids to prevent increases in the blood phosphorous levels, administration of polystyrene suflonate to treat high potassium levels, or dialysis. Acute renal failure may also be successfully treated with recombinant proteins such as human hepatocyte growth factor (HGF) (Goto et al., 1997, Nephron, 77:440). Human alpha-galactosidase A will prevent the progressive deposition of neutral glycosphingolipids in vascular endothelial cells that causes renal failure (Ohshima et al., 1997, Proc. Natl. Acad. Sci. USA,94:2540-2544) and may be useful for the treatment of acute renal failure.

Another recombinant protein called OP-1 (U.S. Pat. No. 5,650,276 and U.S. Pat. No. 5,707,810) is found to protect against kidney damage in animal models of acute and chronic renal failure and may be useful for the treatment of these disorders. OP-1 has been shown to improve the blood flow and filtration in kidneys, thereby reducing toxin accumulation in the bloodstream. OP-1 also reduces the level of expression of certain markers of inflammation. In an animal model of renal failure, a portion of the kidney is removed from nude mice in a two-step nephrectomy procedure in order to simulate a renal failure scenario (Hamamori et al., 1995, J. Clinical Investigation, 95:1808-1813)

The slow, progressive, and irreversible loss of kidney function that is associated with chronic kidney failure, causes an increase in the level of nitrogenous waste products in the blood. Symptoms are slow to develop in an individual suffering from chronic renal failure and can include increased urination, high blood pressure, possibly leading to stroke or heart failure. During the later stages of kidney failure, an increase in the level of toxic substances in the blood can cause fatigue, nerve and muscle symptoms (e.g. twitching and muscle weakness), seizures, digestive tract abnormalities, ulcers and skin disorders. Blood tests that detect increased levels of urea and creatinine or a state of acidosis can be used to diagnose chronic renal failure. Most methods of treating chronic renal failure cannot prevent the progression of this disease. In an individual with chronic renal failure, sodium, water and acid imbalances should be corrected, substances that are toxic to the kidney should be removed, and heart failure, high blood pressure, infections, increased levels of blood potassium or calcium and obstructed urine flow should be treated. If these modes of treatment are ineffective, long-term dialysis or kidney transplantation may be considered as appropriate methods of treatment (Andreoli et al., supra and Berkow et al., supra).

Severe Combined Immunodeficiency Disease (SCID)

SCID results from a deficiency in immunocompetent T and B cells, resulting in severe and persistent infections beginning in the early stages of life. About half of all SCID patients harbor a deficiency in the purine salvage enzyme, adenosine deaminase (ADA). These patients have single base pair mutations in the ADA gene that result in amino acid substitutions, and, in some cases, either a splicing mutation or a deletion (Hirschorn, 1990, Immunodeficiency Review, 2:175-198). Treatment of this form of recessive SCID with adenosine deaminase (ADA) injections is possible. Some SCID patients have an X-linked mutation in the IL-R gamma chain, and treatment of this disease with IL-2 and IL-2R gamma chain may prove to be successful (Leonard et al., 1994, Immunology Review, 138:61-86). Animal models of SCID include a canine model of XSCWD, the most common form of human SCID in the United States, and an equine model of an autosomal recessive form of SCID, (Felsburg et al., Immunodeficiency Review, 3:277-303). Other animal models for SCID include SCED mice and nude mice (Ye and Chiang et al., 1998, Clin. Exp. Rheum., 16:33 and Sandhu et al., 1996, Crit. Rev. Biotechnol., 16:95).

Vaccination

Vaccination is a commonly used method for creating a state of immunity against a specific disease in an individual. Vaccinations can comprise i. dead organisms that retain antigenicity but are no longer capable of inducing disease (useful for treating typhoid fever, whooping cough, diphtheria and other bacterial diseases), ii. toxins that have been chemically treated such that they are antigenic but non-toxic (useful for treating tetanus, botulism, and other toxic diseases), or iii. live organisms that have been mutated such that they do not cause disease but remain antigenic (useful for protection against poliomyelitis, yellow fever, measles, smallpox, and other viral diseases (Guyton, supra).

Whooping cough is a respiratory infection caused by Bordetella pertussis, an organism which produces a wide array of factors that contribute to the development of the disease. The expression and regulation of these virulence factors is dependent upon the bvg locus (originally designated the vir locus), which encodes two proteins: BvgA, a 23-kDa cytoplasmic protein, and BvgS, a 135-kDa transmembrane protein (Merkel et al., 1998, Journal of Bacteriology, 180: 1682-90). Immunization against whooping cough with acellular Bordetella pertussis fragments can confer future protection against whooping cough Ryan et al., 1998, Immunology, 93: 1). Mice with specific disruptions in their B-cell genes (gamma interferon receptor, interleukin 4, or immunoglobulin heavy-chain genes) are shown to be a reliable animal model for studying whooping cough vaccination (Mills et al., 1998, Infectious Immunology, 66:594-602). The murine respiratory challenge model is also a useful model for studying whooping cough vaccination. This model has been used to examine the local T cell responses in the lung during infection with Bordetella pertussis (McGuirk et al., 1998, Eur-J-Immunol., 28: 153-63).

Multiple Sclerosis

Multiple sclerosis (MS) is a central nervous system disease characterized by plaques of demyelination in nerve fibers of the brain and spinal cord. Demyelination causes multiple and varied neurologic symptoms and signs such as neurologic dysfunction including abnormal movement, abnormal sensations, tingling and numbness, loss of strength or dexterity, and visual abnormalities. The physical manifestations of multiple sclerosis result from the demyelination process slowing or blocking the conduction of nerve impulses. MS is typically characterized by periods of relapses and remissions, and eventually becomes progressive in most patients. Although the etiology of multiple sclerosis is not known, it is thought that this disease is caused by both immunologic and genetic factors. The most sensitive method for diagnosing multiple sclerosis is magnetic resonance imaging to detect a loss of myelin as white matter lesions located in the brain and/or spinal cord (Berkow et al., supra)

Currently methods exist for treating the symptoms of multiple sclerosis rather than the disease. The frequency of relapses associated with multiple sclerosis can be decreased with beta-interferon treatment. Beta-interferon also reduces the rate of appearance of cerebral demyelinating lesions. Corticosteroids have also been used to treat multiple sclerosis (Berkow et al., supra). Another protein that may be useful for the treatment of multiple sclerosis is the neuroprotectant molecule annexin-1, a calcium-dependent phospholipid binding protein. A useful animal model for MS is provided by female SJL/J mice with experimental autoimmune encephalomyelitis (EAE), a disease that exhibits symptoms that mimic MS (Ding et al., 1998, J. Immunol., 160: 2560-2564).

Autoimmune Disorders

In some instances, individuals can suffer a loss of immune tolerance to some of their own tissues. Often this results from destruction of some of the body's tissues leading to release of antigens, their circulation in significant quantities in the body fluids, and the production of antibodies directed against these antigens. Autoimmune diseases are characterized by the abnormal production of antibodies reactive against self components.

Diseases that result from autoirnmunity include autoimmune hemolytic anemia caused by the production of antibodies against the bodies own erythrocytes, rheumatic fever wherein exposure to a specific type of streptococcal toxin causes the body to become immunized against tissues in the heart and joints, acute glomerulonephritis wherein exposure to a streptococcal toxin causes an individual to become immunized against the glomeruli, myasthenia gravis wherein the body develops an immunity to muscles that subsequently results in paralysis, and lupus erythenmatosus wherein an individual becomes immunized against multiple tissues simultaneously and suffers extensive damage, often resulting in rapid death (Guyton, supra).

G. Infectious Disease

The invention provides methods of treating infectious diseases including but not limited to Hepatitis C.

Hepatitis C

Hepatitis refers to acute or chronic disorders resulting from liver damage caused by viral, toxic, pharmacologic or immune-mediated factors. All forms of hepatitis share the pathologic features of hepatocellular necrosis and inflammatory cell infiltration of the liver. These changes to the liver may be manifested as an enlarged liver or an increase in the level of transaminase. The symptoms of acute viral hepatitis often appear suddenly and can include gastrointestinal abnormalities, darkened urine, jaundice and symptoms associated with reduced bile flow. Although chronic hepatitis is typically asymptomatic, and rarely causes major liver damage, cirrhosis and liver failure can occur as a result of some cases of chronic hepatitis.

One form of viral hepatitis, known as Hepatitis C, is caused by a flavivirus-like RNA agent. Hepatitis C virus can be identified as the causal agent of chronic or acute hepatitis by diagnostic tests that detect viral proteins or antibodies specific for the virus in the blood. Hepatitis C is a common cause of chronic hepatitis.

Hepatitis C virus (HCV) is a major cause of liver disease worldwide with an estimated occurrence of 150,000 to 170,000 new cases annually in the United States. Currently, it is estimated that about 3.9 million Americans have been infected with HCV. The leading cause of liver transplantation in adults is HCV, due to the damage it causes. HCV is transmitted primarily through inoculations and blood transfusions, although vertical transmission has also been documented. HCV has a high rate of progression (greater than 50%) to chronic disease and eventual cirrhosis. Chronic hepatitis C is characterized by several histological features in the liver which discriminate it from other forms of hepatitis, including bile duct damage, lymphoid follicles and fatty change.

Interferons are the only FDA-approved treatment for hepatitis C, and various types of interferons (e.g interferon-alpha) have been used clinically to treat HCV infections with varying degrees of success (Terranova et al., 1996, Control Clin Trials 17:123-129 and Montalto et al., 1998, Am J Gastroenterol., 93:950-953). It has also been found that two effective ribozymes (CR2 and CR4) can inhibit the expression of a cotransfected reporter gene containing HCV RNA target sequences (Welch et al., 1996, Gene Ther., 3:994-1001); and these results suggest that hairpin ribozymes may be useful for methods of treating HCV infection that involve gene therapy. Interferon treatment is characterized by low response rates and dose-limiting side effects. The effectiveness of interferon treatment has been improved by administering other agents such as thymosin alpha 1 in combination with interferon (Sherman et al., 1998, Hepatology, 27:1128-1135).

Chimpanzees and rodents have provided animal models for studying HCV infection in humans. Several features of human HCV infection are found in the chimpanzee model, including the frequency of persistent infection, and virus replication which occurs despite evidence of cellular and humoral immune responses (Walker et al., 1998, Springer Semin. Immunopathol., 19:85-98). However, although chimpanzees provide a useful model for studying HCV infection, they are not the most practical animals to work with. Efforts have therefore been made to develop useful rodent models for HCV.

According to one rodent model, 2-3 day old mice were infected intracerebrally with HCV (Deriabin et al., 1997, Vopr. Virusol.,42:251-253) and subsequently died 12-14 days later. Additionally, two independent transgenic mouse lines carrying the HCV core gene are now established. As these mice develop progressive hepatic stetosis, they provide a useful animal model for the study of pathogenesis in human HCV infection (Moriya et al., 1997, J. Gen. Verol., 78:1527). Another group has used a chimeric mouse model for the induction of hepatitis C viremia, using BNX (beige/nude/X-linked immunodeficient) mice preconditioned by total body irradiation and reconstituted with SCID mouse bone marrow cells. Following transplantation of HCV-infected liver fragments from patients with HCV-RNA-positive sera under the kidney capsule of the chimeric mice, viremia occurred in approximately 25% of these animals (Galun et al., 1995, J. Infect. Dis., 172:25-30).

H. Muscle Wasting and Whole Body Wasting Disorders

The invention also provides methods of treating muscle wasting and whole body wasting disorders.

Muscle Wasting

Muscle wasting is a loss of muscle mass due to reduced protein synthesis and/or accelerated breakdown of muscle proteins, largely as a result of activation of the non-lysosomal ATP-ubiquitin-dependent pathway of protein degradation. Muscle wasting is caused by a variety of conditions including cachexia associated with diseases including various types of cancer and AIDS, febrile infection, denervation atrophy, steroid therapy, surgery, trauma and any event or condition resulting in a negative nitrogen balance. Muscle wasting also occurs following nerve injury, fasting, fever, acidosis and certain endocrinopathies. Muscle wasting can be detected by measuring protein synthesis and or degradation, the level of production of cell damage markers such as creatine kinase, the activity of a heat shock protein promoter, and changes in the level of components of the ubiquitin dependent protein degradation pathway.

Patients with catabolic wasting disease (e.g. cancer cachexia) are in negative nitrogen balance and suffer a significant and life threatening weight loss. Cancer cachexia is characterized by weakness, anorexia, anemia and progressive skeletal muscle wasting. Other causes of wasting are severe bums, trauma, and major surgery. Wasting diseases effect the quality of life, and are associated with a poor response to chemotherapy as well as decreased survival time following chemotherapy (Tamura et al., 1995, Clinical CancerResearch, 1:1353-1358, Bartlett et al., 1994, Cancer, 73:1499-1504, Tisdale, 1997, Journal of National Cancer Institute, 89: 1763-1773). It is currently hypothesized that the mechanism responsible for the development of cancer cachexia involves production of inflammatory cytokines, which in turn orchestrate a series of complex interrelated steps that ultimately lead to a chronic state of wasting, malnourishment, and death. In an animal model of catabolic wasting diseases, Lewis/Wistar rats are subcutaneously inoculated with the MAC-33 tumor, a spontaneously metastasizing mammary adenocarcinoma. The metastasis of the MAC-33 tumor causes weight loss in the rat and ultimate death. Treatment of these rats with growth hormone, insulin and/or somatostatin resulted in increased body weight and muscle size, as compared to control animals that experienced weight loss over the same period (Bartlett et al.,supra).

I. Neurological Disorders

The invention also provides methods of treating neurological disorders, including peripheral neuropathy, injury, and neurodegenerative diseases (e.g. Parkinson's disease, Huntington's disease or Alzheimer's disease).

Peripheral Neuropathy/Injury

Peripheral neuropathy refers to a malfunction of the peripheral nerves that can disrupt sensation, muscle activity or the function of internal organs. Peripheral neuropathy can involve damage to a single nerve (mononeuropathy), two or more nerves (multiple mononeuropathy) or multiple nerves simultaneously (polyneuropathy). Mononeuropathy is most commonly caused by physical injury and includes carpal tunnel syndrome, ulnar nerve palsy, radial nerve palsy and peroneal nerve palsy. Polyneuropathy is caused by numerous factors including bacterially produced toxins, autoimmune reactions, toxic agents, cancer, nutritional deficiencies and metabolic disorders. Chronic polyneuropathy can result from a number of disorders including diabetes, kidney failure, and malnutrition and the treatment of polyneuropathy depends on the cause (Berkow et al., supra).

Neuronal Disease and Injury

Every year, hundreds of thousands of patients are treated for neurodegenerative disease (e.g. Parkinson's disease, Huntington's Disease, Alzheimer's, multiple sclerosis) or traumatic injury. Damage to the Peripheral Nervous System (PNS) and the Central Nervous System (CNS) can lead to serious disability and death. Therefore, PNS and CNS damage and the attendant social and economic costs are staggering. The adult PNS retains some capacity for regeneration following injury but the return of function in the clinical setting is quite variable and motor and sensory deficits (paralysis, weakness, numbness, etc.) invariably persist (Dyck and Thomas, eds. Peripheral Neuropathy, 3rd. Ed., 1993; W. B. Saunders, Philadelphia, Pa.). In certain situations wherein neuropathy is caused by an underlying disease, such as diabetes or is a drug-induced neuropathy, or in cases where extensive damage has occurred due to severe nerve defects or crush and avulsion injuries, recovery is negligible. Repair of the diseased or damaged CNS, which includes the brain and spinal cord, represents an even greater challenge since almost all disease and injuries lead to an irreversible loss of function (memory loss, loss of motor function, etc.) (Bjorklund et al., eds., 1990, Brain Repair, Stockton Press, New York, N. Y.). New strategies to optimize and enhance regeneration include the delivery of growth-promoting molecules, generally called nerve growth factors.

Delivery of Nerve Growth Factors:

Growth or neuronotrophic factors produced by support cells (e.g. Schwann cells, oligodendrocytes) or by target organs (e.g. muscle fibers, connected neurons) ensure the survival and general growth of neurons. Some factors support neuronal survival, others support nerve outgrowth, and some do both. Numerous growth factors have been identified, cloned, and some have been synthesized through recombinant technologies (Barde, 1989, Neuron 2:1525). The clinical use of such agents has been limited by an inability to deliver the growth factors to the nervous system in the appropriated dose and over an appropriate time period. Methods of administering growth factors by single or multiple injections of growth factors have disadvantages including early burst release, poor control over local drug levels, and significant side effects. A tissue-based delivery system offers the advantages of allowing for controlled regulation of the rate and amount of factor release and maintaining delivery for an extended time period (several months or longer) if needed (e.g. for degenerative diseases such as Parkinson's)

Growth factors useful for the following include: neural repair-neural factors: NGF—nerve growth factor; Neuronal survival, Axon-Schwann cell interaction-BDNF—brain-derived neurotrophic factor; Neuronal survival-CNTF—ciliary neuronotrophic factor Neuronal survival-GDNF—glia- derived neurotrophic factor, Neuronal survival GGF—glial growth factor Schwann cell mitogen NT-3—neurotrophin 3 Neuronal survival NT-4/5-neurotrophin 4/5 Neuronal survival-General factors: IGF-1—insulinlike growth factor 1 Axonal growth; Schwann cell migration-IGF-2—insulinlike growth factor 2; Motoneurite sprouting, muscle reinnervation PDGF—platelet-derived growth factor Cell proliferation, neuronal survival aFGF—acidic fibroblast growth factor; Neurite regeneration, cell proliferation bFGF—basic fibroblast growth factor; Neurite regeneration, neovascularisation Tissue-based delivery may also be used for the concurrent release of growth factors which preferentially control the survival and outgrowth of motor and sensory neurons. For example, NGF and b-FGF control sensory neuronal survival and outgrowth and brain derived growth factor (BDGF) and ciliary neuronotrophic factor (CNTF) control motor neuronal survival and outgrowth. Other molecules, NT-3 and NT 4/5 may carry out both functions. Factors which promote Schwann cell proliferation (e.g. glial growth factor, GGF) may also be useful in enhancing nerve growth. Growth factors released in a sustained, physiologic manner by tissue-based implants may allow regeneration in cases where large nerve deficits exist and in sites where regeneration does not normally occur (e.g. brain and spinal cord).

Animal Models for PNS and CNS Repair

Numerous animal models for neural disease have been developed. Nerves of the PNS can be cut or crushed in a model of nerve transection or neuropathy. It has been demonstrated that nerve guidance channels designed to slowly release basic fibroblast growth factor (bFGF) or nerve growth factor (NGF) can support regeneration over a critical nerve gap in a rat model (Aebischer et al., 1989, J. Neurosci. Res., 23:282-289, Derby et al., 1993, Exp. Neurol., 119:176-191).

In the CNS nerve structures can be cut or chemical substances can be administered to achieve neural damage (Emerich et al., 1994, Neuro. Methods, 21:65-133, Aebischer et al., 1994, Exp. Neurol., 126: 151-158).

J. Skin Disorders

The invention also provides methods of treating skin disorders including wound healing and ulcers.

Wound Healing

Wound healing involves a complex process of cell migration and proliferation, synthesis of extracellular matrix, angiogenesis and remodeling of the collagenous framework that requires many growth factors, such as TGF-beta and platelet-derived growth factor (Amento et al., 1991, Ciba Foundation Symposium, 157: 115-123, Hosgood et al., 1993, Vet. Surg., 226: 490-495. Rat and rabbit animal models for wound healing have been demonstrated (Terrell et al., 1993, International Review Exp Pathology, 34 Pt B: 43-67).

Ulcers

An ulcer is a hole that extends through tissue such as the muscularis mucosa into the submucosa (or a deeper layer) of the gastrointestinal tract. The combined action of acid and pepsin is more injurious to vulnerable mucosa than that of either agent alone. Smoking, stress, heredity factors, aspirin/non-steroidal anti-inflammatory drugs and/or infection with Campylobacter pylori are known to cause peptic ulcers (Chopra et al., 1989, Pathophysiology of Gastrointestinal Diseases). Treatment of peptic ulcers with recombinant proteins such as epidermal growth factor (EGF) may assist in protecting, repairing and healing gastroduodenal mucosa. In an animal model of ulcers, acetic acid has been used to ulcerate rats (Uchida et al., 1989, Japan Journal of Pharmacology, 50:366-368). Ulcers can also be formed in other tissues such as nonhealing skin ulcers in diabetic patients and venous ulcers (Nath et al., 1998, Acta Haematol., 99:175 and Vowden, 1998, J. Wound Care 7:143).

### EXAMPLES

### Example 1

Tissue-Engineered Primary Mouse Myoblast BAMs Express Biologically Active VEGF In Vitro

VEGF Retroviral Vector Construction

Recombinant human VEGF165 (rhVEGF) cDNA (gift of Dr Jeffrey M. Isner, St Elizabeth's Medical Center, Boston, Mass.) was subcloned into the BAM H1 site of pLgXSN12 (gift of Dr Dusty Miller, Fred Hutchinson Cancer Center, Seattle, Wash.). pMFG-mVEGF, an MFG retroviral construct containing the cDNA encoding recombinant murine VEGF 164, was a gift of Dr Helen M. Blau (Stanford University, Palo Alto, Calif.). Recombinant human growth hormone (rhGH) cDNA was used as a soluble, secretable marker of gene activity. It was excised from the MFG-hGH retroviral construct (gift of Dr Jeffrey Morgan, Shriners Bum Institute, Cambridge, Mass.) and subcloned into pLgXSN as described above for rhVEGF165.

Generation of Replication-Deficient Retroviral Producer Cell Lines

Retroviral producer cell lines were generated for LghVEGF165SN, LghGHSN, and LgXSN after a 2-step transfection/transduction protocol optimized for primary adult mouse myoblasts by use of E86 ecotropic and PT67 amphotropic packaging cells. Virus-containing medium was collected from high-titer PT67 clones and stored at −80° C. pMFG-mVEGF was transfected into Phoenix packaging cells (gift of Dr Garry Nolan, Stanford University) to generate virus-containing medium containing mVEGF retrovirus, and β-galactosidase retroviral medium was collected from a stably transduced packaging cell line (CRE BAG 2; CRL-1858, ATCC).

Primary Mouse Myoblast Culture, Transduction, and Tissue-Engineering Into BAMs

Primary mouse myoblasts were isolated from the hind limbs of 4- to 6-week-old male C3HeB/FeJ mice (Jackson Laboratory, Bar Harbor, Me.) and maintained in culture according to standard procedures (Powell, C. et al., Gene Therapy Protocols, Humana Press; in press; Pinset, C. et al., 1996, Cell Biology: A Laboratory Handbook 2nd ed, 1: 226). Isolated cells were transduced with polybrene-supplemented virus-containing medium according to a centrifugation protocol (Springer, M. et al., 1997, Somat Cell Mol Genet., 23: 203). BAMs for subcutaneous implants were formed from 2×106 transduced myoblasts and were 1×15 mm, (Shansky, J. et al., 1997, In Vitro Cell Dev Biol Anim., 33: 659) whereas those implanted into ischemic hind limbs were 10 mm long and were formed from 1.5×10⁶ myoblasts. BAMs were treated with cytosine arabinoside (1 [g/mL) for 3 to 6 days before implantation to eliminate proliferating cells as previously described. (Vandenburgh, H. et al., 1996, Hum Gene Ther. 7: 2195).

Transduced primary mouse skeletal myoblasts were tissue-engineered into BAMs by suspending the cells in a collagen-Matrigel extracellular matrix solution and casting the suspension into silicone rubber molds with artificial end attachment points (Shansky, J. et al., 1997, In Vitro Cell Dev Biol Anim., 33: 659). Internal longitudinal tensions develop within the cell-gel mixture as it dehydrates, causing the formation of a cylindrical structure 1 mm in diameter and containing parallel arrays of multinucleated postmitotic myofibers. Hematoxylin-eosin staining of BAM cross sections revealed no morphological difference between rVEGF and control BAMs (data not shown).

Western blotting of culture medium from rhVEGF BAMs under reducing conditions showed 2 bands with molecular weights of 28 and 23 kDa, with the majority of the secreted protein in the 28-kDa band; rhVEGF standards showed a major band at 26 kDa and a minor band at 23 kDa (data not shown). The BAMs in vitro secreted consistent levels of hVEGF (428 to 579 ng·BAM−1·d⁻¹), mVEGF (156 to 456 ng·BAM−1·d⁻¹), or hGH (6.0 to 8.2 μg·BAM−1·d⁻¹). for several weeks (data not shown). BAMs formed from nontransduced myoblasts secreted 5 to 13 ng mVEGF·BAM−1·d⁻¹, and no detectable hVEGF (<0.03 ng BAM−1·d⁻¹). rhVEGF BAMs (n=3, 14 days in vitro) were assayed for tissue levels of VEGF. Each BAM contained a mean of 23±3 ng hVEGF, indicating that 95% of hVEGF synthesized by the BAM was secreted into the medium over a 24-hour period. Similar in vitro results were found for rmVEGF BAMs (data not shown).

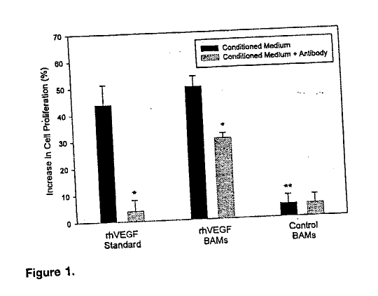

The biological activity of secreted rhVEGF was determined by its ability to increase endothelial cell proliferation. HWVECs were incubated with conditioned medium from either rhVEGF BAMs (rhVEGF concentration of 10 ng/mL) or control BAMs. The mitogenic activity on HUVECs increased 50±4% with conditioned medium from rhVEGF BAMs, compared with an increase of 5±3% with medium from control BAMs relative to unsupplemented medium (FIG. 1). The growth response elicited by rhVEGF-conditioned medium was only partially neutralized by an antibody specific for hVEGF, probably because of the synergistic stimulation of HUVEC proliferation by other growth factors present in the conditioned medium (e.g., insulin-like growth factor-1). FIG. 1 demonstrates that rhVEGF secreted by rhVEGF BAMs is biologically active. HUVECs were incubated overnight with either 10 ng/mL rhVEGF165 standard or conditioned medium collected from rhVEGF or control BAMs. Medium was diluted 1:10 and added to HUVEC cultures in presence or absence of 8 μg/mL anti-human VEGF 165 antibody, n=4 wells/group. *P<0.01 standard rhVEGF vs standard+antibody, and rhVEGF BAM vs rhVEGF BAM+antibody; **P<0.0001 control BAM vs rhVEGF BAM.

Statistical Analyses

Results are expressed as mean±SEM, and comparisons were by unpaired t tests, with P<0.05 taken as a statistically significant difference.

### Example 2

BAMs Implanted Subcutaneously Into Syngeneic Mice Can Survive for at Least 5 Weeks In Vivo

Surgical Procedures: Implantation of BAMs and Ischemic Model

All experimental animal procedures were approved by the Institutional Animal Care and Use Committee and conformed to the guiding principles of the American Physiological Society. After 14 days in vitro, BAMs were implanted into 4- to 6-week-old male C3HeB/FeJ mice. Mice receiving rhVEGF or rhGH BAM implants were immunosuppressed with cyclosporine (60 mg/kg daily) because of the potential for formation of antibodies to the human protein. Subcutaneous BAM implants were as previously described (Vandenburgh, H., 1998, Hum Gene Ther, 9: 2555) with either 1 BAM (rVEGF implants) or 2 BAMs (rhGH implants) implanted into the back of each animal. For the ischemic model, the femoral and saphenous arteries were ligated in 1 hind limb of each mouse, and side branches were removed (Couffinhal, T. et al., 1998, Am J Pathol, 152: 1667). One BAM was implanted in the ischemic hindlimb between the tibialis anterior muscle fascia and overlying skin and secured in place on the fascia by fibrin sealant (Tisseel V H; Baxter Hyland) to maintain myofiber tension.

Tissue Histochemistry and Quantification of Capillary Density in BAMs

BAM and host muscle explants were either frozen in isopentane or fixed with 0.25% glutaraldehyde for cryostat sectioning. Capillary density was examined by quantification of endothelial cells in cryostat sections stained with anti-mouse CD31 (Pharmingen), an antibody specific for mouse endothelial cells, following standard immunoperoxidase procedures and development with DAB. The primary antibody was omitted from negative controls. Five nonoverlapping microscopic fields were analyzed from each explanted BAM by use of the Zeiss KS 300 Version 3.0 Image Analysis System, and the area that stained positive for CD31 was quantified and expressed as a percentage of the total area analyzed.

For β-gal staining, glutaraldehyde-fixed BAMs were cryosectioned and stained with an X-gal Substrate Set (Kirkegaard & Perry Laboratories).

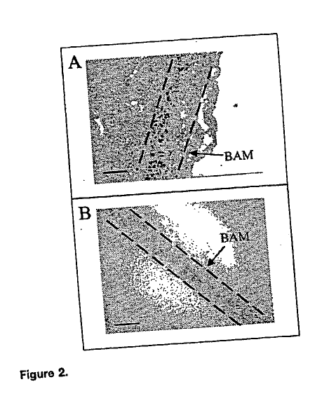

rmVEGF-BAG BAMs and BAG BAMs were implanted subcutaneously into syngeneic mice. Explanted BAMs showed areas of healthy myofibers that stained β-gal-positive after 1 to 5 weeks in vivo, with no β-gal staining outside the area of the implant (FIG. 2), indicating that the transduced cells in the BAMs have not migrated from the implant site.

FIG. 2 demonstrates that postmitotic myofibers in subcutaneously implanted BAMs survive in vivo for up to 5 weeks. rmVEGF-BAG BAMs were explanted and stained for β-gal after 1 week (A) and 5 weeks (B) in vivo. A, Cross sections were counterstained with hematoxylin-eosin. Bar=100 μm.

### Example 3

mVEGF Levels are Greater in Implanted rmVEGF BAMs than in Control BAMs

Growth Factor Analyses

mVEGF and rVEGF protein levels in culture medium from BAMs and mouse serum were measured with ELISA kits (R&D Systems). The minimum detectable dose with these kits is 3 to 5.0 pg/mL. To measure tissue levels of extracellular matrix-bound mVEGF or hVEGF, BAMs were homogenized in protein lysis buffer (Lee, L. et al., 2000, Ann Thorac Surg., 69: 14). Total protein was measured by the BCA protein assay (Pierce). hGH levels in culture medium and serum were assayed by a radioimmunoassay technique that does not cross-react with mouse GH (Vandenburgh, H. et al., 1996, Hum Gene Ther. 7: 2195). For Western blots, aliquots of conditioned culture medium containing 6 ng of hVEGF165 were subjected to electrophoresis on 12% SDS-polyacrylamide gels, transferred to a nitrocellulose membrane, probed with anti-hVEGF (sc152, Santa Cruz Biotechnology), and developed with ECL detection reagent (Amersham).

Stimulation of human umbilical vein endothelial cell (HUVEC, Clonetics) proliferation by conditioned medium from BAMs was used as a measure of VEGF bioactivity (Witzenbichler, B. et al., 1998, Am J Pathol. 153: 381). Anti-hVEGF monoclonal antibody (R&D) was added to some culture wells, and rhVEGF165 (R&D, 10 ng/mL) served as a positive control.

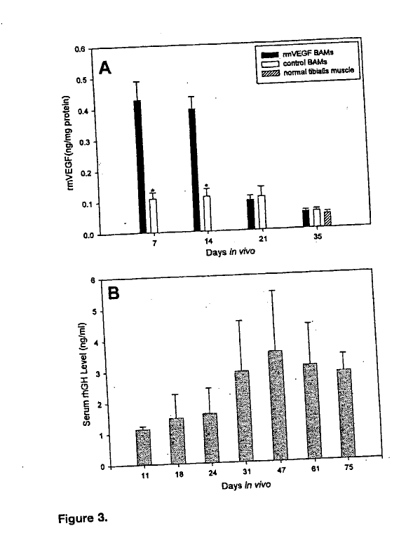

One and 2 weeks after implantation, the mVEGF content of rmVEGF BAMs was 2.5- to 3.0-fold higher than in control BAMs (FIG. 3A). In BAMs explanted after 3 and 5 weeks, the level of mVEGF in rmVEGF BAMs was similar to that of control BAMs and comparable to levels in normal mouse tibialis anterior muscle (FIG. 3A). This decrease after 2 weeks in vivo is not due to cell death or promoter inactivation, because rhGH BAMs engineered with the same LgXSN construct and from the same primary myoblasts expressed soluble hGH for >10 weeks (FIG. 3B).

FIG. 3 demonstrates that mVEGF levels are elevated within implanted rmVEGF BAMs vs control BAMs for up to 2 weeks, whereas hGH is detectable in serum with rhGH BAMs implanted for >10 weeks. rmVEGF BAMs and control BAMs (A) were implanted subcutaneously into normal mice for 1, 2, 3, and 5 weeks. Explanted BAM mVEGF levels were measured in tissue homogenates as described above. rhGH BAMs (B) were implanted subcutaneously into normal mice, and serum hGH levels were measured every 1 to 2 weeks (n=2 to 4 per group). *P<0.05.

### Example 4

Vascularization is Accelerated within VEGF-Secreting RAMs

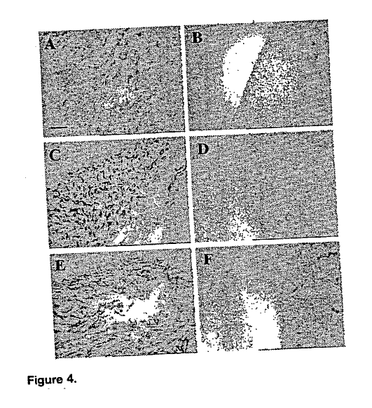

Capillary in growth into subcutaneously implanted BAMs showed a significantly higher density of CD31-positive cells in mVEGF BAMs than in control BAMs at all time points (FIG. 4). FIG. 4 demonstrates that angiogenesis is increased in subcutaneously implanted rmVEGF BAMs. rmVEGF BAMs (A, C, E) and control BAMs (B, D, F) were explanted after 1 week (A, B), 3 weeks (C, D), or 5 weeks (E, F), and immunostained with antibody against CD31/PECAM-1 to identify mouse endothelial cells. Bar=100 μm.

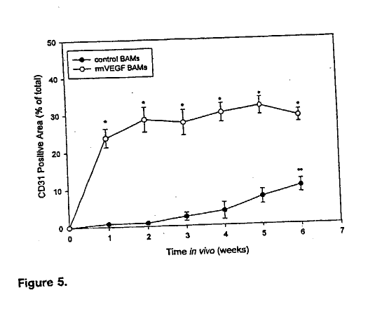

After 1 week, 23.8±2.5% of the total cross-sectional area stained positive for CD31 in rmVEGF BAMs, compared with only 0.8±0.2% in nonsecreting BAMs (FIG. 5). FIG. 5 is a time course of angiogenesis in implanted BAMs. Endothelial cell densities were quantified as outlined in Methods in rmVEGP and control BAMs, and area staining positive for CD31 was expressed as % of total area analyzed, n=20. *P<0.0001, rmVEGF BAMs vs control BAMs; **P<0.0001 control BAMs, 6 weeks vs 1 week.

This increase was sustained out to 6 weeks (28.9±1.7% versus 10.1±1.8% in rmVEGF-secreting and control BAMs, respectively). Similar results were observed in short-term studies of immunosuppressed mice implanted with rhVEGF-secreting BAMs, but longer-term implant studies were not performed with rhVEGF BAM implants because of the high level of immunosuppressant required. In no instance did hemangiomas form in or around the rVEGF BAM implants.

### Example 5

Angiogenesis of Ischemic Muscle is Accelerated by rmVEGF BAMs

Capillary in growth in the ischemic tibialis anterior muscle was significantly increased as early as 1 week in mice receiving rmVEGF-secreting implants compared with control BAMs or no implants and continued to increase for up to 4 weeks (FIG. 6). Capillary density in the tibialis anterior muscle was greatest near the implant at 1 week, but by 4 weeks there was no difference along the length of the muscle (data not shown). FIG. 6 is a time course of capillary density in ischemic tibialis of mice receiving rmVEGF BAMs, nonsecreting BAMs, or no implants. Endothelial cell densities in tibialis muscle in ischemic hindlimbs were quantified by CD31 staining. *P<0.0001, rmVEGF BAMs vs control BAMs.

### Example 6

Systemic Levels of mVEGF are not Increased by rmVEGF BAM Implants

Serum levels of mVEGF were assayed from mice implanted with rmVEGF BAMs, control BAMs, and mice with no implants. There were no significant differences between any of the groups in mice receiving either subcutaneous or ischemic hindlimb implants for up to 6 weeks (38.6 to 56.1 pg/mL for both control and rmVEGF implants), demonstrating that rmVEGF BAMs act locally rather than systemically to stimulate angiogenesis.

### Example 7

Vascularization of Implanted BAMs can be Predicted from Preimplant mVEGF Secretion Levels

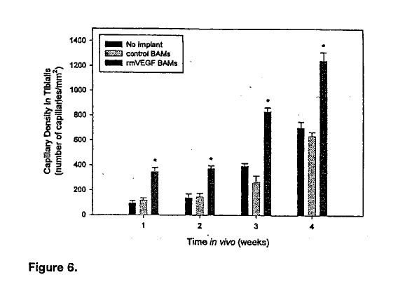

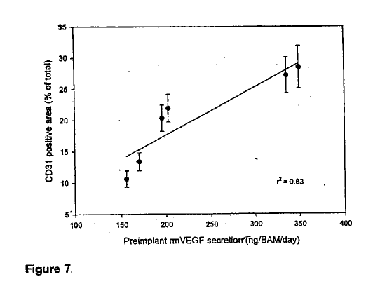

One advantage to using BAMs as a delivery platform for a foreign gene product is the ability to monitor protein secretion levels before implantation (Vandenburgh, H., 1998, Hum Gene Ther, 9: 2555). To determine whether it is possible to accurately predict the in vivo biological effect of mVEGF from in vitro mVEGF secretion levels, we compared the secretion level from preimplant rmVEGF BAMs (156 to 336 ng mVEGF BAM⁻¹·d⁻¹) to their capillary density after implantation for 3 weeks (FIG. 7). FIG. 7 demonstrates rmVEGF BAM stimulation of capillary growth in vivo is predictable from preimplantation in vitro secretion levels. BAMs secreting various levels of rmVEGF before implantation were explanted after 3 weeks and quantified by CD31 staining, n=20.

A linear relationship exists between the area staining positive for CD31 and preimplantation secretion levels of the BAMs (r2=0.83), indicating that stimulation of capillary growth by rmVEGF BAMs in vivo can be predicted from preimplantation in vitro mVEGF secretion levels.

Primary adult mouse myoblasts can be genetically engineered to secrete rhVEGF or rmVEGF and tissue-engineered into bioartificial muscles (BAMs). rhVEGF BAMs secreted hVEGF165 with molecular weights of 28 and 23 kDa. Similar results have been reported in other studies (Seghezzi, G. et al., 1998, J Cell Biol., 141: 1659) and may represent glycosylation variants of rhVEGF 165. Bioactivity of rhVEGF secreted from BAMs was demonstrated by its ability to stimulate the growth of human umbilical vein endothelial cells in vitro. Subcutaneous implantation of rhVEGF- or rmVEGF-secreting BAMs into syngeneic mice resulted in significantly increased vascularization of rVEGF-secreting BAMs compared with nonsecreting BAMs, confirming the bioactivity of the secreted rmVEGF and rhVEGF in vivo. In addition, implantation of rmVEGF-secreting BAMs into an ischemic hindlimb stimulated localized angiogenesis of neighboring host muscle tissue. These results suggest that tissue-engineered skeletal muscle may be a practical platform to secrete biologically active rVEGF in order to stimulate angiogenesis in neighboring ischemic tissue.

rVEGF gene therapy has been shown to promote therapeutic angiogenesis in preclinical models of tissue ischemia (Asahara, T. et al., 1997, Science, 275: 964; Takeshita, S. et al., 1994, Circulation, 90(5 pt 2): II-228) and in human clinical trials (Isner, J. et al., 1996, Lancet, 348: 370; Baumgartner, I. et al., 1998, Circulation, 97: 1114; Losordo, D. et al., 1998, Circulation, 98: 2800). Therapeutic angiogenesis is not risk-free, however. Some possible negative side effects using various methods of rVEGF delivery are the production of nonfunctional leaky vessels and enhancement of vascular permeability, (Dvorak, H. et al., 1995, J Pathol., 146: 1029) development of hemangiomas, (Springer, M. et al., 1998, Mol Cell., 2: 549; Lee, R. et al., 2000, Circulation., 102: 898) and the stimulation of angiogenesis in tumors (Folkman, J., 1995, N Engl J Med., 333: 1757). It is therefore important to determine a means of optimally inducing localized angiogenesis with minimal effects systemically and to find an appropriate dose of rVEGF that minimizes the potential deleterious effects on nearby tissue (Springer, M. et al., 1998, Mol Cell., 2: 549).

Delivery of rVEGF from BAMs is shown in the present study to target a local area with no elevation in serum levels, no harmful effects on neighboring tissue, and no hemangioma formation for up to 6 weeks in vivo. In another study, implantation of rmVEGF-engineered proliferating myoblasts into nonischemic mouse leg muscles or the heart led to hemangiomas within 6 weeks (Springer, M. et al., 1998, Mol Cell., 2: 549; Lee, R. et al., 2000, Circulation., 102: 898). The different results in the present study may be due to different pharmacokinetics and/or localized tissue structure of the implant site (intramuscular in the myoblast study (Springer, M. et al., 1998, Mol Cell., 2: 549) versus subcutaneous/intermuscular in the present study). rVEGF delivery from injected myoblasts may result in different physiological effects than when delivered from implants formed from myoblasts fused ex vivo into postmitotic muscle fibers. Passage of primary mouse myoblasts beyond 35 to 40 doublings has been found (Irintchev, A. et al., 1998, J Cell Sci., 111: 3287) to lead to their spontaneous transformation into immortalized cells that continue to proliferate when implanted in vivo. This may be one cause of uncontrolled angiogenesis in mouse models (Springer, M. et al., 1998, Mol Cell., 2: 549; Lee, R. et al., 2000, Circulation., 102: 898). Such immortalization has not been seen in human myoblasts (Powell, C. et al., 1999, Hum Gene Ther., 10: 565).

The use of retroviral vectors in our studies resulted in the stable integration of the rVEGF gene into the host cell genome and long-term expression when implanted in vivo. Adenoviral vectors are characterized by a progressive loss of gene expression, because they are not integrated into the host genome (Lee, L. et al., 2000, Ann Thorac Surg., 69: 14; Powell, C. et al., 1999, Hum Gene Ther., 10: 565) mVEGF levels in explanted BAMs were significantly elevated after 1 and 2 weeks in vivo but decreased to that of normal mouse skeletal muscle by 3 to 4 weeks (FIG. 3A). In contrast, hGH secretion from BAMs genetically engineered with the same retroviral construct persists for months (FIG. 3B). It is not known why the BAM mVEGF levels decrease. Myofibers survive adequately in the BAM for 5 weeks on the basis of β-gal staining (FIG. 2), so decreased secretion due to myofiber death is unlikely. Possibly a feedback mechanism exists, such that once the blood supply is increased into the “ischemic” BAM, the myofibers no longer synthesize rmnVEGF. The LTR promoter may be shutting off, an explanation that seems unlikely, because β-gal gene expression, also driven by the LTR promoter, persists in the implants for >5 weeks. It seems most likely that once the BAMs are well vascularized, rmVEGF is still expressed but is rapidly delivered to localized host tissue by the newly formed blood vessels and no longer accumulates in the BAM itself.

One advantage of implanting genetically engineered postmitotic myofibers is that secretion levels of growth factors can be monitored in vitro, before implant surgery. In a previous study, we showed that in vivo systemic levels of rhGH from implanted BAMs could be predicted from preimplantation secretion levels (Vandenburgh, H., 1998, Hum Gene Ther, 9: 2555). We demonstrate here that biological activity of mVEGF secreted from BAMs can also be predicted from in vitro secretion levels (FIG. 7), because higher mVEGF secretion levels resulted in a higher density of capillary growth. Protein delivery by injected myoblasts or by intramuscular plasmid DNA injection is limited by the variability in the number of postmitotic muscle fibers that take up and express the foreign gene, making secretion levels difficult to predict.⁹ With BAM technology, the desired in vivo biological effect can be regulated by engineering BAMs with varying numbers of growth factor-secreting myofibers or by implanting varying numbers of BAMs into each animal (Vandenburgh, H., 1998, Hum Gene Ther, 9: 2555).

Cell-based delivery of rVEGF from a “living protein delivery platform” composed of fused, postmitotic muscle cells results in the stimulation of endothelial cell growth into the subcutaneous implants in mice and increases capillary growth into nearby host muscle in an ischemic hindlimb model. Extending rVEGF BAM technology to human skeletal muscle offers great potential for the treatment of ischemic disease. Human adult skeletal muscle cells isolated from elderly congestive heart failure patients and genetically engineered to secrete rhGH can be formed into rhGH-secreting BAMs (Powell, C. et al., 1999, Hum Gene Ther., 10: 565). The subsequent implantation of human BAMs for gene therapy would offer the advantage of a predictable delivery platform having a high protein synthesis capacity and long-term survival (decades for skeletal myofibers).

### Example 8

Delivery of VEGF from an Organized Tissue Promotes Angiogenesis

Delivery of recombinant vascular endothelial growth factor (rVEGF) from tissue-engineered bioartificial muscles (BAMs) was investigated as a novel strategy to promote localized therapeutic angiogenesis. Primary adult mouse myoblasts retrovirally transduced to secrete rVEGF were suspended in an extracellular matrix and cast into silicon molds. The cell/matrix mixture gelled to form cylindrical 1 mm×10-15 mm mouse BAMs (mBAMs) containing parallel arrays of myofibers. Subcutaneous implantation of rVGEF rnBAMs (in vitro secretion of 290-511 ng VEGF/BAM/day) into syngeneic mice resulted in a 30-fold increase in vascularization of neighboring host muscle tissue by one week that was maintained for four weeks with no evidence of hemangioma formation. No elevation of serum-VEGF occurred with either implant site. In related preliminary studies, myoblasts from adult sheep transduced to secrete rVEGF were engineering into 1 mm×20 mm ovine (oBAMs), with VEGF-secretion levels of 30-148 ng/BAM/day. Control and rVEGF-oBAMs were implanted into hearts of normal autologous sheep. The oBAMs were either fibrin-glued to a pericardial patch material and sutured to the left ventricle epicardium, or fibrin-glued into the atrioventricular groove. In one-week implants, the myocardial area surrounding rVEGF-oBAMs appeared to be more highly vascularized than areas under control implants. (Data not shown).

### Example 9

Production of a Vascularized Organized Tissue by the Addition of a vasculogenic Factor to the Extracellular Matrix

An organized tissue according to the invention is prepared from cells that comprise or do not comprise a recombinant nucleic acid encoding a vasculogenic factor, as described in Example 1. A vascuogenic factor, for example, regranex, or PDGF-BB, is added to the extracellular matrix (for example the collagen-Matrigel, when it is in a liquid state at 4° C.

### Example 10

Production of a Vascularized Organized Tissue by the Post-Implantation Addition of a vasculogenic Factor to an Organism

An organized tissue is prepared from cells comprising a recombinant nucleic acid encoding a vasculogenic factor as described in Example 1 and implanted as described in Example 2. Prior to closing up the implantation site, a vasculogenic factor (for example regranex or PDGF-BB is added (by injection or sprinkling) to the implantion site. Vascularization is determined according to the methods described herein.

1. A method of delivering a bioactive compound to an organism comprising the steps of:
growing in vitro a plurality of cells;
wherein at least a subset of cells comprises a DNA sequence selected from the group consisting of a DNA sequence encoding a vasculogenic factor, a DNA sequence encoding a vasculogenic factor operably linked to a promoter and a DNA sequence encoding a factor that increases the expression of a vasculogenic factor, and
at least a subset of cells comprises a bioactive compound to be delivered to said organism; and
wherein said cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce;
culturing said coalesced suspension under conditions in which said cells connect to said attachment surfaces and fonn an organized tissue having an in vivo-like gross and cellular morphology that is retained upon retrieval of said organized tissue; and
implanting said tissue into said organism, wherein said organized tissue becomes vascularized; and whereby said bioactive compound is produced and delivered to said organism, whereby said bioactive compound is of a type or produced in an amount not produced by said tissue of interest, wherein said bioactive compound is produced sufficiently to provide a therapeutic effect to said organism upon implantation of said organized tissue into said organism.

2. The method of claim 1, wherein said organized tissue is comprised of substantially post-mitotic cells.

3. The method of claim 1, wherein said organized tissue has an in vivo-like gross and cellular morphology of said tissue of interest.

4. The method of claim 1, wherein said vasculogenic factor is selected from the group consisting of: VEGF A, VEGF B, VEGF C, VEGF D, VEGF E, VEGF F, FGF 1, FGF 2, FGF 3, FGF 4, FGF-5, PDGF AA, PDGF BB, PDGF AB, angiopoeitin, MCP, EPO, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22

5. The method of claim 1, wherein said implantation is performed by a subcutanteous method.

6. The method of claim 1, further comprising the steps of removing said organized tissue from said organism to terminate delivery of the bioactive compound.

7. The method of claim 6 further comprising, following said removal step the step of: culturing said tissue in vitro under conditions which preserve its in vivo viability.

8. The method of claim 7 further comprising, following said culturing step: the step of:
reimplanting said tissue into said organism to deliver said bioactive compound to said organism.

9. The method of claim 1, wherein said tissue is implanted into the tissue of origin of at least one of said cells.

10. The method of claim 1, wherein at least a subset of cells comprises a DNA sequence that mediates the production of two proteins.

11. The method of claim 1, wherein said bioactive compound is a protein.

12. The method of claim 11 wherein said protein is a growth factor.

13. The method of claim 11, wherein said protein is unstable.

14. The method of claim 11, wherein said protein is Factor VIII.

15. The method of claim 1, wherein said organized tissue is comprised of at least one of a cell type selected from the group consisting of: skeletal muscle cells, myoblasts, myofibers, fibroblasts, endothelial cells, smooth muscle cells, cardiac myocytes, osteoblasts, neuronal cells, hepatocytes, mesenchymal stem cells, marrow-derived stem cells, adult stem cells and embryonic stem cells

16. The method of claim 1, wherein during said growing step, a force is exerted parallel to a dimension of the tissue.

17. The method of claim 1, wherein a force is exerted on the individual cells during growth in vitro and on said organized tissue during implantation in vivo.

18. The method of claim 1 wherein said tissue comprises skeletal muscle.

19. The method of claim 1 wherein said tissue comprises myotubes.

20. The method of claim 1 wherein said cells comprise myofibers.

21. The method of claim 1 wherein said organism is a mammal.

22. The method of claim 1, wherein said mammal is a human.

23. A method of delivering a bioactive compound to an organism comprising the steps of:
growing in vitro a plurality of cells;
wherein at least a subset of cells comprises a bioactive compound to be delivered to said organism; and
wherein said cells are mixed with an extracellular matrix to create a suspension, and further mixed with at least one vasculogenic factor;
placing said suspension in a vessel having a three-dimensional geometry approximating the in vivo gross morphology of a tissue of interest, said vessel having attachment surfaces thereon; and
allowing said suspension to coalesce;
culturing said coalesced suspension under conditions in which said cells connect to said attachment surfaces and form an organized tissue having an in vivo-like gross and cellular morphology that is retained upon retrieval of said organized tissue; and
implanting said tissue into said organism, wherein said organized tissue becomes vascularized; and whereby said bioactive compound is produced and delivered to said organism, whereby said bioactive compound is of a type or produced in an amount not produced by said tissue of interest, wherein said bioactive compound is produced sufficiently to provide a therapeutic effect to said organism upon implantation of said organized tissue into said organism.

24. A method of delivering a bioactive compound to an organism comprising the steps of:
growing in vitro a plurality of cells wherein at least a subset of cells comprises a bioactive compound to be delivered to said organism; and
wherein said cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce;
culturing said coalesced suspension under conditions in which said cells connect to said attachment surfaces and form an organized tissue having an in vivo-like gross and cellular morphology that is retained upon retrieval of said organized tissue; and
implanting said tissue into said organism and adding at least one vasculogenic factor to said organism, wherein said organized tissue becomes vascularized; and whereby said bioactive compound is produced and delivered to said organism, whereby said bioactive compound is of a type or produced in an amount not produced by said tissue of interest, wherein said bioactive compound is produced sufficiently to provide a therapeutic effect to said organism upon implantation of said organized tissue into said organism.

25. A method of delivering a bioactive compound to an organism comprising the steps of: growing in vitro a plurality of cells,
wherein at least a subset of cells comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor, and
wherein at least a subset of the cells comprises a bioactive compound, and
wherein said cells are mixed with an extracellular matrix to create a suspension;
placing said suspension in a vessel wherein said cells form an organized tissue of interest having a three dimensional cellular organization which is retained upon implantation into said organism; and
implanting said organized tissue into said organism, whereby said organized tissue is vascularized; and wherein said bioactive compound is produced and delivered to said organism sufficiently to provide a therapeutic effect to said organism, whereby said bioactive compound is of a type or produced in an amount not produced by said tissue of interest.

26. A method of delivering a bioactive compound to an organism comprising the steps of: growing in vitro a plurality of cells,
wherein at least a subset of cells comprises a bioactive compound, and
wherein said cells are mixed with an extracellular matrix to create a suspension and further mixed with at least one vasculogenic factor;
placing said suspension in a vessel wherein said cells form an organized tissue of interest having a three dimensional cellular organization which is retained upon implantation into said organism; and
implanting said organized tissue into said organism, whereby said organized tissue is vascularized; and wherein said bioactive compound is produced and delivered to said organism sufficiently to provide a therapeutic effect to said organism, whereby said bioactive compound is of a type or produced in an amount not produced by said tissue of interest.

27. A method of delivering a bioactive compound to an organism comprising the steps of: growing in vitro a plurality of cells,
wherein at least a subset of cells comprises a bioactive compound, and
wherein said cells are mixed with an extracellular matrix to create a suspension;
placing the suspension in a vessel wherein said cells form an organized tissue of interest having a three dimensional cellular organization which is retained upon implantation into said organism;
implanting said organized tissue into said organism, and adding at least one vasculogenic factor to said organism, whereby said organized tissue is vascularized; and wherein said bioactive compound is produced and delivered to said organism sufficiently to provide a therapeutic effect to said organism, whereby said bioactive compound is of a type or produced in an amount not produced by said tissue of interest.

28. A method of providing a bioactive compound to an organism in therapeutic need thereof comprising: implanting into the organism an organized tissue having a three-dimensional geometry that is retained upon retrieval of said organized tissue,
wherein at least a subset of cells comprise a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor, and
wherein at least a subset of cells of the organized tissue comprises a bioactive compound to be delivered to said organism, and
wherein said bioactive compound is produced sufficiently to provide a therapeutic effect to said organism upon implantation of the organized tissue into said organism, and
wherein the implanted organized tissue is vascularized.

29. A method of providing a bioactive compound to an organism in therapeutic need thereof comprising:
implanting into said organism an organized tissue having a three-dimensional geometry that is retained upon retrieval of said organized tissue,
wherein at least a subset of cells comprise a bioactive compound to be delivered to the organism,
wherein said organized tissue is produced by mixing said cells with an extracellular matrix to create a suspension, and further mixing said cells with at least one vasculogenic factor, and
wherein said bioactive compound is produced sufficiently to provide a therapeutic effect to the organism upon implantation of the organized tissue into the organism and,
wherein said implanted organized tissue is vascularized.

30. A method of providing a bioactive compound to an organism in therapeutic need thereof comprising:
implanting into said organism an organized tissue having a three-dimensional geometry that is retained upon retrieval of said organized tissue,
wherein at least a subset of cells comprises a bioactive compound to be delivered to the organism, wherein at least one vasculogenic factor is added to said organism following implantation,
wherein said implanted organized tissue is vascularized, and
wherein said bioactive compound is produced sufficiently to provide a therapeutic effect to said organism upon implantation of said organized tissue into said organism.

31. A method of providing a bioactive compound to an organism in therapeutic need thereof comprising:
implanting into said organism an organized tissue having a three-dimensional geometry approximating the in vivo gross morphology of a tissue of interest that is retained upon implantation of said organized tissue into said organism,
wherein at least a subset of the cells of the organized tissue comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor, and
wherein at least a subset of the cells of the organized tissue comprises a bioactive compound, and
wherein said bioactive compound is produced sufficiently to provide a therapeutic effect to said organism upon implantation of said organized tissue into said organism, and
wherein said implanted organized tissue is vascularized.

32. A method of providing a bioactive compound to an organism in therapeutic need thereof comprising:
implanting into said organism an organized tissue having a three-dimensional geometry approximating the in vivo gross morphology of a tissue of interest that is retained upon implantation of said organized tissue into said organism,
wherein at least a subset of the cells of the organized tissue comprises a bioactive compound, wherein said organized tissue is produced by mixing said cells with an extracellular matrix to create a suspension and further mixing said cells with at least one vasculogenic factor, and wherein said bioactive compound is produced sufficiently to provide a therapeutic effect to said organism upon implantation of said organized tissue into said organism, and
wherein said implanted organized tissue is vascularized,.

33. A method of providing a bioactive compound to an organism in therapeutic need thereof comprising:
implanting into said organism an organized tissue having a three-dimensional geometry approximating the in vivo gross morphology of a tissue of interest that is retained upon implantation of said organized tissue into said organism,
wherein at least a subset of the cells of the organized tissue comprises a bioactive compound,
wherein at least one vasculogenic factor is added to said organism after said implantation, and wherein said bioactive compound is produced sufficiently to provide a therapeutic effect to said organism upon implantation of said organized tissue into said organism, and wherein said implanted organized tissue is vascularized,.

34. An in vitro method for producing an organized tissue which has an in vivo-like gross and cellular morphology and is vascularized following implantation into an organism, comprising the steps of:
providing cells of said tissue, wherein at least a subset of said cells comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor, wherein the cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce; and
culturing said coalesced suspension under conditions in which said cells connect to said attachment surfaces and form an organized tissue having a three dimensional structure that is retained upon retrieval of the organized tissue, wherein at least a subset of the cells of the organized tissue comprise said DNA sequence encoding said vasculogenic factor.

35. The method of claim 34, wherein said organized tissue further comprises a subset of cells comprising a bioactive compound.

36. The method of claim 34, wherein said organized tissue is comprised of substantially post-mitotic cells.

37. The method of claim 34, wherein said organized tissue has an in vivo-like gross and cellular morphology of said tissue of interest.

38. The method of claim 34, wherein the step of providing comprises isolating primary cells of at least one of the cell types comprising said tissue of interest.

39. The method of claim 34, wherein the step of providing comprises utilizing immortalized cells of at least one of the cell types comprising said tissue.

40. The method of claim 34, wherein prior to the step of providing, a foreign DNA sequence operably linked to a promoter and encoding a protein is introduced to at least a subset of said cells.

41. The method of claim 34, wherein said cells comprise skeletal muscle cells.

42. The method of claim 34, wherein said coalesced suspension exerts a force on said cells parallel to a dimension of said vessel.

43. The method of claim 34, wherein said cells are aligned parallel to a dimension of said vessel.

44. The method of claim 43, wherein said attachment surfaces are positioned at opposite ends of said vessel.

45. The method of claim 34, wherein said organized tissue produces said protein.

46. An in vitro method for producing an organized tissue which has an in vivo-like gross and cellular morphology and is vascularized following implantation into an organism, comprising the steps of:
providing cells of said tissue, wherein the cells are mixed with an extracellular matrix to create a suspension and further mixed with at least one vasculogenic factor;
placing said suspension in a vessel having a three-dimensional geometry approximating the in vivo gross morphology of a tissue of interest, said vessel having attachment surfaces thereon; and
allowing said suspension to coalesce; and
culturing said coalesced suspension under conditions in which said cells connect to said attachment surfaces and form an organized tissue having a three dimensional structure that is retained upon retrieval of the organized tissue.

47. The method of claim 46, wherein said organized tissue further comprises a subset of cells comprising a bioactive compound.

48. An in vitro method for producing an organized tissue which has an in vivo-like gross and cellular morphology and is vascularized following implantation into an organism, comprising the steps of:
providing cells of said tissue, wherein the cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce; and
culturing said coalesced suspension under conditions in which said cells connect to said attachment surfaces and form an organized tissue having a three-dimensional structure that is retained upon retrieval of the organized tissue, and
wherein at least one vasculogehic factor is added to said organism following said implantation.

49. The method of claim 48, wherein said organized tissue further comprises a set of cells comprising a bioactive compound.

50. An organized tissue having an in vivo gross cellular morphology and producing a protein of a type or produced in an amount not produced normally by a tissue of interest, produced according to the method of claim 34.

51. An organized tissue producing a bioactive compound of a type or produced in an amount not produced normally by a tissue of interest, where said organized tissue is produced by the steps of:
mixing a plurality of cells with an extracellular matrix to create a suspension,
wherein at least a subset of said cells comprises a DNA sequence selected from the group consisting of a DNA sequence encoding a vasculogenic factor, a DNA sequence encoding a vasculogenic factor operably linked to a promoter or a DNA sequence encoding a factor that increases the expression of a vasculogenic factor and further comprises a bioactive compound;
placing said suspension in a vessel having a three-dimensional geometry approximating the in vivo gross morphology of the tissue of interest, the vessel having attachment surfaces thereon; and
allowing said suspension to coalesce; and
culturing said coalesced suspension under conditions in which said cells connect to said attachment surfaces and form an organized tissue having a three-dimensional structure that is retained upon retrieval of said organized tissue from said organism, and wherein said organized tissue is vascularized following implantation into said organism; and wherein said bioactive compound is produced at detectable levels in said tissue.

52. The organized tissue of claim 51, further comprising substantially post-mitotic cells.

53. The organized tissue of claim 51, wherein said organized tissue comprises an in vivo-like gross and cellular morphology of said tissue of interest.

54. The organized tissue of claim 51, wherein said tissue is skeletal muscle.

55. An organized tissue producing a bioactive compound of a type or produced in an amount not produced normally by a tissue of interest, where the organized tissue is produced by the steps of:
mixing a plurality of cells with an extracellular matrix to create a suspension, and
further mixing said cells with at least one vasculogenic factor,
wherein at least a subset of said cells comprises a bioactive compound;
placing said suspension in a vessel having a three-dimensional geometry approximating the in vivo gross morphology of the tissue of interest, the vessel having attachment surfaces thereon; and
allowing said suspension to coalesce; and
culturing said coalesced suspension under conditions in which said cells connect to said attachment surfaces and form an organized tissue having a three dimensional structure that is retained upon retrieval of said organized tissue from said organism, and wherein said organized tissue is vascularized following implantation into said organism; and wherein said bioactive compound is produced at detectable levels in said tissue.

56. An organized tissue producing a bioactive compound of a type or produced in an amount not produced normally by a tissue of interest, where the organized tissue is produced by the steps of:
mixing a plurality of cells with an extracellular matrix to create a suspension,
wherein at least a subset of said cells comprises a bioactive compound;
placing said suspension in a vessel having a three-dimensional geometry approximating the in vivo gross morphology of the tissue of interest, the vessel having attachment surfaces thereon; and
allowing said suspension to coalesce; and
culturing said coalesced suspension under conditions in which said cells connect to said attachment surfaces and form an organized tissue having a three dimensional structure that is retained upon retrieval of said organized tissue from said organism,
implanting said organized tissue into said organism and adding at least one vasculogenic factor to said organsim, and
wherein said organized tissue is vascularized following implantation into said organism; and
wherein said bioactive compound is produced at detectable levels in said tissue.

57. An organized tissue having an in vivo-like gross and cellular morphology of a tissue of interest and producing a bioactive compound of a type or produced in an amount not produced normally by said tissue of interest comprising:
a plurality of cells, wherein at least a subset of the cells comprise a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor, and further comprising a bioactive compound,
wherein said cells form an organized tissue has a three-dimensional structure that is retained upon retrieval of the organized tissue from said organism, and
wherein the organized tissue is vascularized following implantation into an organism; and wherein said bioactive compound is produced at detectable levels in the tissue.

58. The organized tissue of claim 57, wherein said organized tissue comprises substantially post-mitotic cells.

59. The organized tissue of claim 57, wherein the organized tissue approximates the in vivo gross morphology of the tissue of interest.

60. An organized tissue having an in vivo-like gross and cellular morphology of a tissue of interest and producing a bioactive compound of a type or produced in an amount not produced normally by said tissue of interest comprising:
a plurality of cells, wherein at least a subset of the cells comprises a bioactive compound,
wherein said cells form an organized tissue having a three-dimensional structure that is retained upon retrieval of the organized tissue from said organism, wherein said organized tissue is formed by mixing said cells with an extracellular matrix to create a suspension, and further mixing said cells with at least one vasculogenic factor, and
wherein said organized tissue is vascularized following implantation into an organism; and;
wherein said bioactive compound is produced at detectable levels in the tissue.

61. An organized tissue having an in vivo-like gross and cellular morphology of a tissue of interest and producing a bioactive compound of a type or produced in an amount not produced normally by said tissue of interest comprising:
a plurality of cells, wherein at least a subset of the cells comprises a bioactive compound,
wherein said cells form an organized tissue having a three-dimensional structure that is retained upon retrieval of the organized tissue from said organism,
wherein at least one vasculogenic factor is added to said organism following implantation, wherein said organized tissue is vascularized following implantation into an organism; and;
wherein said bioactive compound is produced at detectable levels in the tissue.

62. An organized tissue producing a protein produced by the steps of:
mixing a plurality of mammalian cells, wherein at least a subset of the cells comprises a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor and
further comprises a bioactive compound,
wherein said cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce; and
culturing said coalesced suspension under conditions in which said cells connect to the attachment surfaces, wherein said suspension of cells forms an organized tissue that has a three-dimensional structure that is retained upon implantation of the tissue into a mammal, and
wherein said tissue is vascularized upon implantation into an organism, and
wherein said bioactive compound is produced sufficiently to provide a therapeutic effect to said organism once said organized tissue is implanted into said organism.

63. The organized tissue of claim 62, further comprising substantially post-mitotic cells.

64. An organized tissue producing a protein produced by the steps of:
mixing a plurality of mammalian cells, wherein at least a subset of the cells comprises a bioactive compound, wherein the cells are mixed with an extracellular matrix to create a suspension and further mixed with at least one vasculogenic factor;
placing said suspension in a vessel having a three-dimensional geometry approximating the in vivo gross morphology of a tissue of interest, the vessel having attachment surfaces thereon; and
allowing said suspension to coalesce; and
culturing said coalesced suspension under conditions in which said cells connect to said attachment surfaces, wherein the suspension of cells forms an organized tissue that has a three-dimensional structure that is retained upon implantation of the tissue into a mammal, and wherein the tissue is vascularized upon implantation into an organism, and
wherein the bioactive compound is produced sufficiently to provide a therapeutic effect to the organism once the organized tissue is implanted into the organism.

65. An organized tissue producing a protein produced by the steps of:
mixing a plurality of mammalian cells, wherein at least a subset of the cells comprises a bioactive compound, wherein the cells are mixed with an extracellular matrix to create a suspension;
placing said suspension in a vessel having a three-dimensional geometry approximating the in vivo gross morphology of a tissue of interest, the vessel having attachment surfaces thereon; and
allowing said suspension to coalesce; and
culturing said coalesced suspension under conditions in which said cells connect to said attachment surfaces, wherein the suspension of cells forms an organized tissue that has a three-dimensional structure that is retained upon implantation of the tissue into a mammal, implanting said organized tissue into said organism, adding at least one vasculogenic factor to said organism; and
wherein said tissue is vascularized upon implantation into said organism, and
wherein said bioactive compound is produced sufficiently to provide a therapeutic effect to said organism once said organized tissue is implanted into said organism.

66. An organized tissue having a three-dimensional cellular organization of a tissue of interest that is retained upon implantation of the tissue into an organism, the tissue producing a bioactive compound of a type or in an amount not normally produced by a tissue of interest, comprising:
a plurality of cells, wherein at least a subset of said cells comprises a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor and
further comprising a bioactive compound,
wherein said organized tissue has a three-dimensional structure that is retained upon retrieval of said tissue from said organism, and
wherein said organized tissue is vascularized following implantation into said organism; and
wherein said bioactive compound is produced to detectable levels in said tissue of interest.

67. The organized tissue of claim 66, wherein said organized tissue comprises substantially post-mitotic cells.

68. The organized tissue of claim 66, wherein said organized tissue has a three-dimensional geometry approximating the in vivo gross morphology of said tissue of interest.

69. An organized tissue having a three-dimensional cellular organization of a tissue of interest that is retained upon implantation of the tissue into an organism, the tissue producing a bioactive compound of a type or in an amount not normally produced by a tissue of interest, comprising:
a plurality of cells, wherein at least a subset of said cells comprises a bioactive compound,
wherein said organized tissue is formed by mixing said cells with an extracellular matrix to create a suspsension and further mixing with at least one vasculogenic factor,
wherein said tissue has a three-dimensional geometry that is retained upon retrieval of said tissue from said organism, and
wherein said organized tissue is vascularized following implantation into said organism; and wherein said bioactive compound is produced to detectable levels in said tissue of interest.

70. An organized tissue having a three-dimensional cellular organization of a tissue of interest that is retained upon implantation of the tissue into an organism, the tissue producing a bioactive compound of a type or in an amount not normally produced by a tissue of interest, comprising:
a plurality of cells, wherein at least a subset of said cells comprises a bioactive compound,
wherein said tissue has a three-dimensional geometry that is retained upon retrieval of said tissue from said organism,
wherein said organized tissue is implanted into said organism and
wherein at least one vasculogenic factor is added to said organism following implantation; and
wherein said organized tissue is vascularized following implantation into said organism; and
wherein said bioactive compound is produced to detectable levels in said tissue of interest.

71. An organized tissue attached to a surface of a substrate, the tissue producing a bioactive compound, comprising:
a plurality of cells, wherein at least a subset of the cells comprise a DNA sequence selected from the group consisting of a DNA sequence encoding a vasculogenic factor, a DNA sequence encoding a vasculogenic factor that is operably linked to a promoter or a DNA sequence encoding a factor that increases the expression of a vasculogenic factor and
further comprising a bioactive compound,
wherein the cells form an organized tissue having a three-dimensional geometry that is retained upon retrieval of the organized tissue from an organism into which it has been implanted, and
wherein said organized tissue is attached to the surface of a substrate, and
wherein said organized tissue is vascularized upon implantation into an organism, and;
wherein the bioactive compound is produced to detectable levels in the tissue of interest.

72. The organized tissue of claim 71, wherein said organized tissue comprises substantially post-mitotic cells.

73. The organized tissue of claim 71, wherein said organized tissue comprises an in vivo gross morphology of said tissue of interest.

74. The organized tissue of claim 71, said substrate being selected from the group consisting of metal or plastic.

75. The organized tissue of claim 74 said metal substrate being steel mesh having a longitudinal axis and first and second points for attachment, and wherein said first and second attachment sites of said tissue are atached, respectively, to said first and second points of attachment.

76. An organized tissue attached to a surface of a substrate, the tissue producing a bioactive compound, comprising:
a plurality of cells, wherein at least a subset of the cells comprises a bioactive compound,
wherein said organized tissue is formed by mixing said cells with an extracellular matrix to create a suspension and further mixing with at least one vasculogenic factor,
wherein the cells form an organized tissue having a three-dimensional geometry approximating the in vivo gross morphology of a tissue of interest and that is retained upon retrieval of the organized tissue from an organism into which it has been implanted, and
wherein said organized tissue is attached to the surface of a substrate, and
wherein said organized tissue is vascularized upon implantation into an organism, and;
wherein said bioactive compound is produced to detectable levels in the tissue of interest.

77. An organized tissue attached to a surface of a substrate, the tissue producing a bioactive compound, comprising:
a plurality of cells, wherein at least a subset of the cells comprises a bioactive compound,
wherein the cells form an organized tissue having a three-dimensional geometry approximating the in vivo gross morphology of a tissue of interest and that is retained upon retrieval of the organized tissue from an organism into which it has been implanted, and
wherein said organized tissue is attached to the surface of a substrate,
implanting said organized tissue and adding at least one vasculogenic factor to said organism following implantation, and
wherein said organized tissue is vascularized upon implantation into an organism, and;
wherein said bioactive compound is produced to detectable levels in the tissue of interest.

78. A method of delivering a vasculogenic factor to an organism comprising the steps of:
growing in vitro a plurality of cells;
wherein at least a subset of cells comprises a DNA sequence encoding a vasculogenic factor or a DNA sequence encoding a vasculogenic factor operably linked to a promoter, and
wherein said cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce;
culturing said coalesced suspension under conditions in which said cells connect to said attachment surfaces and form an organized tissue having an in vivo-like gross and cellular morphology that is retained upon retrieval of said organized tissue; and
implanting said tissue into said organism, wherein said organized tissue becomes vascularized; and whereby said vasculogenic factor is produced and delivered to said organism, whereby said vasculogenic factor is of a type or produced in an amount not produced by said tissue of interest, wherein said vasculogenic factor is produced sufficiently to provide a therapeutic effect to said organism upon implantation of said organized tissue into said organism.

79. A method of delivering a vasculogenic factor to an organism comprising the steps of: growing in vitro a plurality of cells,
wherein at least a subset of cells comprises a DNA sequence encoding a vasculogenic factor, or a DNA sequence encoding a vasculogenic factor operably linked to a promoter, and
wherein said cells are mixed with an extracellular matrix to create a suspension;
placing said suspension in a vessel wherein said cells form an organized tissue of interest having a three dimensional cellular organization which is retained upon implantation into said organism; and
implanting said organized tissue into said organism, whereby said organized tissue is vascularized; and wherein said vasculogenic factor is produced and delivered to said organism sufficiently to provide a therapeutic effect to said organism, whereby said vasculogenic factor is of a type or produced in an amount not produced by said tissue of interest.

80. A method of providing a vasculogenic factor to an organism in therapeutic need thereof comprising: implanting into the organism an organized tissue having a three-dimensional geometry that is retained upon retrieval of said organized tissue,
wherein at least a subset of cells comprise a DNA sequence encoding a vasculogenic factor or a DNA sequence encoding a vasculogenic factor operably linked to a promoter, and
wherein at least a subset of cells of the organized tissue comprises a vasculogenic factor to be delivered to said organism, and
wherein said vasculogenic factor is produced sufficiently to provide a therapeutic effect to said organism upon implantation of the organized tissue into said organism, and
wherein the implanted organized tissue is vascularized.

81. A method of providing a vasculogenic factor to an organism in therapeutic need thereof comprising:
implanting into said organism an organized tissue having a three-dimensional geometry approximating the in vivo gross morphology of a tissue of interest that is retained upon implantation of said organized tissue into said organism,
wherein at least a subset of the cells of the organized tissue comprises a DNA sequence encoding a vasculogenic factor or a DNA sequence encoding a vasculogenic factor operably linked to a promoter, and
wherein said vasculogenic factor is produced sufficiently to provide a therapeutic effect to said organism upon implantation of said organized tissue into said organism, and
wherein said implanted organized tissue is vascularized.

82. An organized tissue producing a vasculogenic factor of a type or produced in an amount not produced normally by a tissue of interest, where said organized tissue is produced by the steps of:
mixing a plurality of cells with an extracellular matrix to create a suspension,
wherein at least a subset of said cells comprises a DNA sequence encoding a vasculogenic factor or a DNA sequence encoding a vasculogenic factor operably linked to a promoter;
placing said suspension in a vessel having a three-dimensional geometry approximating the in vivo gross morphology of the tissue of interest, the vessel having attachment surfaces thereon; and
allowing said suspension to coalesce; and
culturing said coalesced suspension under conditions in which said cells connect to said attachment surfaces and form an organized tissue having a three-dimensional structure that is retained upon retrieval of said organized tissue from said organism, and wherein said organized tissue is vascularized following implantation into said organism; and wherein said vasculogenic factor is produced at detectable levels in said tissue.

83. An organized tissue having an in vivo-like gross and cellular morphology of a tissue of interest and producing a vasculogenic factor of a type or produced in an amount not produced normally by said tissue of interest comprising:
a plurality of cells, wherein at least a subset of the cells comprise a DNA sequence encoding a vasculogenic factor or a DNA sequence encoding a vasculogenic factor that is operably linked to a promoter and
wherein said cells form an organized tissue has a three-dimensional structure that is retained upon retrieval of the organized tissue from said organism, and
wherein the organized tissue is vascularized following implantation into an organism; and wherein said vasculogenic factor is produced at detectable levels in the tissue.

84. An organized tissue producing a vasculogenic factor produced by the steps of:
mixing a plurality of mammalian cells, wherein at least a subset of the cells comprises a DNA sequence encoding a vasculogenic factor, or a DNA sequence encoding a vasculogenic factor that is operably linked to a promoter,
wherein said cells are mixed with an extracellular matrix to create a suspension;
placing said suspension in a vessel having a three-dimensional geometry approximating the in vivo gross morphology of a tissue of interest, said vessel having attachment surfaces thereon; and
allowing said suspension to coalesce; and
culturing said coalesced suspension under conditions in which said cells connect to the attachment surfaces, wherein said suspension of cells forms an organized tissue that has a three-dimensional structure that is retained upon implantation of the tissue into a mammal, and
wherein said tissue is vascularized upon implantation into an organism, and
wherein said vasculogenic factor is produced sufficiently to provide a therapeutic effect to said organism once said organized tissue is implanted into said organism.

85. An organized tissue having a three-dimensional cellular organization of a tissue of interest that is retained upon implantation of the tissue into an organism, the tissue producing a vasculogenic factor of a type or in an amount not normally produced by a tissue of interest, comprising:
a plurality of cells, wherein at least a subset of said cells comprises a DNA sequence encoding a vasculogenic factor, or a DNA sequence encoding a vasculogenic factor that is operably linked to a promoter,
wherein said organized tissue has a three-dimensional structure that is retained upon retrieval of said tissue from said organism, and
wherein said organized tissue is vascularized following implantation into said organism; and
wherein said vasculogenic factor is produced to detectable levels in said tissue of interest.
